(19) United States
(12) Patent Application Publication
(10) Pub. No.: US 2023/0024434 A1
(43) Pub. Date: Jan. 26, 2023

(54) Methods for Developing Personalized Drug Treatment Plans and Targeted Drug Development Based on Proteomic Profiles

(71) Applicant: SomaLogic, Inc., Boulder, CO (US)

(72) Inventors: Larry Gold, Boulder, CO (US); Robert Kirk Delisle, Boulder, CO (US); David Sterling, Boulder, CO (US); Rachel Ostroff, Boulder, CO (US); Dom Zichi, Boulder, CO (US)

(73) Assignee: SomaLogic, Inc., Boulder, CO (US)

(21) Appl. No.: 17/358,186

(22) Filed: Jun. 25, 2021

Related U.S. Application Data
(63) Continuation of application No. 15/754,064, filed on Feb. 21, 2018, now abandoned.
(60) Provisional application No. 62/215,852, filed on Sep. 9, 2015.

(51) Int. Cl. G01N 33/68; G01N 33/50

(52) U.S. Cl. CPC G01N 33/68; G01N 33/5011; A61K 31/16; G01N 2800/52; G01N 2500/00

(57) ABSTRACT

The present invention relates to developing customized therapies for a disease or condition in a subject. In particular, the present invention relates to aptamer-based compositions and methods for identifying, modulating and monitoring drug targets in individual with a disease or condition, and further composition and methods for identifying and selecting protein targets for drug development.

### CROSS-REFERENCE TO RELATED APPLICATIONS

This application is a continuation of U.S. application Ser. No. 15/754,064, filed Feb. 21, 2018, which is a national phase entry pursuant to 35 U.S.C. § 371 of International Application No. PCT/US2016/050908, filed Sep. 9, 2016, which claims the benefit of priority of U.S. Provisional Application No. 62/215,852, filed Sep. 9, 2015, each of which is incorporated by reference herein in its entirety for any purpose.

### FIELD

The present invention relates to developing customized therapies for a disease or condition in a subject. In particular, the present invention relates to aptamer-based compositions and methods for identifying, modulating and monitoring drug targets in an individual with a disease or condition, and further composition and methods for identifying and selecting protein targets for drug development.

### BACKGROUND

Oncogenes have become the central concept in understanding cancer biology and may provide valuable targets for therapeutic drugs. In many types of human tumors, including lymphomas and leukemias, oncogenes are over-expressed and may be associated with tumorigenicity (Tsujimoto et al., Science 228:1440-1443 [1985]). For instance, high levels of expression of the human bcl-2 gene have been found in all lymphomas with a t(14; 18) chromosomal translocations including most follicular B cell lymphomas and many large cell non-Hodgkin's lymphomas. High levels of bcl-2 gene expression have also been found in certain leukemias that do not have a t(14; 18) chromosomal translation, including most cases of chronic lymphocytic leukemia acute, many lymphocytic leukemias of the pre-B cell type, neuroblastomas, nasophryngeal carcinomas, and many adenocarcinomas of the prostate, breast and colon. (Reed et al., Cancer Res. 51:6529 [1991]; Yunis et al., New England J. Med. 320:1047; Campos et al., Blood 81:3091-3096 [1993]; McDonnell et al., Cancer Res. 52:6940-6944 [1992); Lu et al., Int. J. Cancer 53:29-35 [1993]; Bonner et al., Lab Invest. 68:43 A [1993]. Other oncogenes include TGF-.alpha., c-ki-ras, ras, her-2 and c-myc.

Gene expression, including oncogene expression, can be inhibited by molecules that interfere with promoter function. Accordingly, the expression of oncogenes may be inhibited by single stranded oligonucleotides.

Cancer treatment typically includes chemotherapeutic agents and often radiation therapy. In many cases, however, the current treatments are not efficacious or do not cure the cancer. Consequently, there is a need for more effective cancer treatments.

For example, lung cancer remains the leading cause of cancer death in industrialized countries. About 75 percent of lung cancer cases are categorized as non-small cell lung cancer (e.g., adenocarcinomas), and the other 25 percent are small cell lung cancer. Lung cancers are characterized in to several stages, based on the spread of the disease. In stage I cancer, the tumor is only in the lung and surrounded by normal tissue. In stage II cancer, cancer has spread to nearby lymph nodes. In stage III, cancer has spread to the chest wall or diaphragm near the lung, or to the lymph nodes in the mediastinum (the area that separates the two lungs), or to the lymph nodes on the other side of the chest or in the neck. This stage is divided into IIIA, which can usually be operated on, and stage TIM, which usually cannot withstand surgery. In stage IV, the cancer has spread to other parts of the body.

Most patients with non-small cell lung cancer (NSCLC) present with advanced stage disease, and despite recent advances in multi-modality therapy, the overall ten-year survival rate remains dismal at 8-10% (Fry et al., Cancer 86:1867 [1999]). However, a significant minority of patients, approximately 25-30%, with NSCLC have pathological stage I disease and are usually treated with surgery alone. While it is known that 35-50% of patients with stage I disease will relapse within five years (Williams et al., Thorac. Cardiovasc. Surg. 82:70 [1981]; Pairolero et al., Ann, Thorac. Surg. 38:331 [1984]), it is not currently possible to identify which specific patients are at high risk of relapse.

Adenocarcinoma is currently the predominant histologic subtype of NSCLC (Fry et al., supra; Kaisermann et al., Brazil Oncol. Rep. 8:189 [2001]; Roggli et al., Hum. Pathol. 16:569 [1985]). While histopathological assessment of primary lung carcinomas can roughly stratify patients, there is still an urgent need to identify those patients who are at high risk for recurrent or metastatic disease by other means. Previous studies have identified a number of preoperative variables that impact survival of patients with NSCLC (Gail et al., Cancer 54:1802 1984]; Takise et al., Cancer 61:2083 [1988]; Ichinose et al., J. Thorac. Cardiovasc. Surg. 106:90 [1993]; Harpole et al., Cancer Res. 55:1995]). Tumor size, vascular invasion, poor differentiation, high tumor proliferate index, and several genetic alterations, including K-ras (Rodenhuis et al., N. Engl. J. Med. 317:929 [1987]; Slebos et al., N. Engl. J. Med. 323:561 [1990]) and p53 (Harpole et al., supra; Horio et al., Cancer Res. 53:1 [1993]) mutation, have been reported as prognostic indicators.

Tumor stage is an important predictor of patient survival, however, much variability in outcome is not accounted for by stage alone, as is observed for stage I lung adenocarcinoma which has a 65-70% five-year survival (Williams et al., supra; Pairolero et al., supra). Current therapy for patients with stage I disease usually consists of surgical resection and no additional treatment (Williams et al., supra; Pairolero et al., supra). The identification of a high-risk group among patients with stage I disease would lead to consideration of additional therapeutic intervention for this group, as well as leading to improved survival of these patients.

There is a need for additional diagnostic and treatment options, particularly treatments customized to a patient's tumor.

### SUMMARY

The present invention relates to customized cancer therapy. In particular, the present invention relates to aptamer-based compositions and methods for identifying, modulating and monitoring drug targets in individual cancers.

For example, in some embodiments, the present disclosure provides a method for identifying protein targets, comprising: a) assaying a biological sample from a subject diagnosed with a disease to identify altered levels of one or more proteins relative to the level of the protein in a reference sample; and b) identifying one or more treatments that targets one or more of the proteins with altered expression. The present disclosure is not limited to particular protein targets. In some embodiments, targets are identified by screening samples for levels of protein expression and comparing the levels to normal (e.g., disease-free) tissue (e.g., using aptamer technology described herein). The invention is not limited by the target identified (e.g., using aptamer technology described herein. In some embodiments, the proteins are selected from, for example, those shown in Tables 6 and 7 or AGER, THBS2, CA3, MMP12, PIGR, DCN, PGAM1, CD36, FABP, ACP5, CCDC80, PPBP, LYVE1, STC1, SPON1, IL17RC, MMP1, CA1, SERPINC1, TPSB2, CKB/CKBM, NAMPT/PBEF, PPBP/CTAPIII, F9, DCTPP1, F5, SPOCK2, CAT, PF4, MDK, BGN, CKM, POSTN, PGLYRP1, or CXCL12. In some embodiments, the reference sample is a sample of normal tissue from the subject, or a population average of normal tissue. In some embodiments, the level of the proteins are altered at least 2-fold (e.g., at least 4-fold, at least 5-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, or more). In some embodiments, the level of the proteins are altered at least fold 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold). In some embodiments, the method further comprises the step of administering the one or more treatments to the subject. In some embodiments, the method further comprises the step of determining the presence of mutations in the proteins. In some embodiments, the disease is, for example, a cancer (e.g., leukemia, lymphoma, prostate cancer, lung cancer, breast cancer, liver cancer, colorectal cancer, kidney cancer, etc.), a metabolic disorder, an inflammatory disease, or an infectious disease. In some embodiments, the biological sample is selected from, for example, tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytological fluid, nipple aspirate, bronchial aspirate, bronchial brushing, synovial fluid, joint aspirate, organ secretions, cells, a cellular extract, or cerebrospinal fluid. In some embodiments, the drug is, for example, those described herein. In some embodiments, the assaying comprises contacting a sample with a plurality of aptamers specific for the proteins.

Further embodiments provide a method for determining a treatment course of action, comprising: a) assaying a tissue sample from a subject diagnosed with cancer (e.g., lung cancer) to identify altered levels of one or more proteins selected from, for example, AGER, THBS2, CA3, MMP12, PIGR, DCN, PGAM1, CD36, FABP, ACP5, CCDC80, PPBP, LYVE1, STC1, SPON1, IL17RC, MMP1, CA1, SERPINC1, TPSB2, CKB/CKBM, NAMPT/PBEF, PPBP/CTAPIII, F9, DCTPP1, F5, SPOCK2, CAT, PF4, MDK, BGN, CKM, POSTN, PGLYRP1, CXCL12, or a protein shown in Table 6 or 7, relative to the level of the proteins in normal tissue (e.g., normal lung tissue); and b) administering one or more treatments that targets one or more of the proteins with altered expression.

Additional embodiment provide a method for treating a disease, comprising: a) assaying a biological sample from a subject diagnosed with a disease to identify altered levels of one or more proteins relative to the level of the protein in a reference sample; and b) administering one or more treatments that target one or more of the proteins with altered expression to the subject.

Further embodiment provide a method for treating a disease, comprising: a) assaying a biological sample from a subject diagnosed with a disease to identify altered levels of one or more proteins relative to the level of the protein in a reference sample; and b) administering one or more treatments that target one or more of the proteins with altered expression to the subject; and c) repeating the step of assaying the biological sample from a subject diagnosed with a disease to identify altered levels of one or more proteins relative to the level of the protein in a reference sample.

Yet other embodiments provide a method for monitoring treating of a disease, comprising: a) assaying a biological sample from a subject diagnosed with a disease to identify altered levels of one or more proteins relative to the level of the protein in a reference sample; b) administering one or more treatments that target one or more of the proteins with altered expression to the subject; and c) repeating step a) one or more times.

Still further embodiments provide a method for screening test compounds, comprising: a) assaying a biological sample from a subject diagnosed with a disease to identify altered levels of one or more proteins relative to the level of the protein in a reference sample; b) administering one or more test compounds that target or are suspected of targeting one or more of the proteins with altered expression to the subject; and c) repeating step a) one or more times.

The foregoing and other objects, features, and advantages of the invention will become more apparent from the following detailed description, which proceeds with reference to the accompanying figures.

### BRIEF DESCRIPTION OF THE DRAWINGS

FIG. 1 depicts a dendrogram showing proteins with at least one example of having a 10-fold change (up or down) for tumor tissue to healthy tissue. The data are clustered based on the change in protein level. The tree is labeled by SampleID:Histology (Adeno/Squamous) to show that the two different tumor types (adenocarcinoma and squamous cell carcinoma) do not separate from each other based on protein levels. SampleID indicates the patient sample.

FIG. 2 depicts a dendrogram showing proteins with at least one example of having a 10-fold change (up or down) for tumor tissue to healthy tissue. The data are clustered based on the change in protein level. The tree is labeled by SampleID:Mutation Status, and shows that the samples do not group by mutational status. WT means that no mutations were found out of those tested. ND means mutation profiling was not performed. Those with no mutation listing means the status is unknown. SampleID indicates the patient sample.

FIG. 3 shows a comparison of mRNA expression levels for adeno or squamous tumors versus the protein levels. The data are derived from two different sources: mRNA expression data had adeno and squamous tumors. mRNA levels were averaged across all studies. Protein expression levels were derived from a separate source. Each point represents a single protein and corresponding mRNA. The box in the middle represents those mRNAs and proteins that were removed because they were not at least 2-fold up or down relative to control for either mRNA level or protein level. The boxed dots are those that were not considered to be significantly different in tumor versus normal for both mRNA and protein.

FIG. 4 shows pictographs generated plotting the relative protein expression levels shown in relative fluorescence units (RFU) vs. age (years) of subjects in both non-Duchene muscular dystrophy (DMD) and DMD boys for several proteins that are different between the control and the DMD subjects.

### DETAILED DESCRIPTION

The present invention relates to customized cancer therapy. In particular, the present invention relates to aptamer-based compositions and methods for identifying, modulating and monitoring drug targets in individual cancers.

The confluence of genomics technologies and the awareness of cancers as diseases driven by somatic and inherited mutations have led to a hope that a combination of pathology and cancer genomics will provide personalized decisions regarding therapeutic interventions. An enormous effort, funded largely by the NCI, will deepen the sequencing of tumor genomes to see major and common drivers of the disease as well as minor groups of cells whose additional somatic mutations will determine prognostics and treatment choices.

Work by others has had a profound impact on the ways one considers tumor genetics. These scientists painstakingly created mouse strains in which transposon mutagenesis is easily induced, and thus driver mutations and subsequent required mutations can be studied for mouse tumor development. The body of work from the Copeland/Jenkins labs is enormous and important. One may conclude from their work that a tumor that requires several mutations on the tumorigenesis pathway can easily suffer those mutations in several different kinetic stages, and single driver mutations can elaborate tumors through different subsequent mutations that take the tumor into different physiological and biochemical states.

The scientific community, through CPTAC, has begun an analysis of tissue proteomics alongside genomics through the TCGA and others. Eight institutions in the United States were funded to do largely Mass Spectrometry as a way into the proteomic phenotypes of cancers, which noted that protein expression was not well correlated with mRNA levels of DNA copy numbers.

Historically cancers have been described as derived from a tissue of origin—lung cancer, prostate cancer, breast cancer, etc. However, to date, it has not been possible to identify, in real time, all of part of a tumor proteome of cancer (e.g., in order to identify and/or characterize protein involvement within individual tumors and cancers).

Embodiments of the present disclosure provide systems and method for identifying proteins with altered expression in individual tumors. The systems and methods provide customized drug targets and individualized therapies for cancer.

### I. Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aptamer: The term aptamer, as used herein, refers to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule.

Analog: The term analog, as used herein, refers to a structural chemical analog as well as a functional chemical analog. A structural chemical analog is a compound having a similar structure to another chemical compound but differing by one or more atoms or functional groups. This difference may be a result of the addition of atoms or functional groups, absence of atoms or functional groups, the replacement of atoms or functional groups or a combination thereof. A functional chemical analog is a compound that has similar chemical, biochemical and/or pharmacological properties. The term analog may also encompass S and R stereoisomers of a compound.

Bioactivity: The term bioactivity, as used herein, refers to one or more intercellular, intracellular or extracellular process (e.g., cell-cell binding, ligand-receptor binding, cell signaling, etc.) which can impact physiological or pathophysiological processes.

C-5 Modified Pyrimidine: C-5 modified pyrimidine, as used herein, refers to a pyrimidine with a modification at the C-5 position. Examples of a C-5 modified pyrimidine include those described in U.S. Pat. Nos. 5,719,273 and 5,945,527. Additional examples are provided herein.

Consensus Sequence: Consensus sequence, as used herein, refers to a nucleotide sequence that represents the most frequently observed nucleotide found at each position of a series of nucleic acid sequences subject to sequence alignment.

Covalent Bond: Covalent bond or interaction refers to a chemical bond that involves the sharing of at least a pair of electrons between atoms.

Modified: The term modified (or modify or modification) and any variations thereof, when used in reference to an oligonucleotide, means that at least one of the four constituent nucleotide bases (i.e., A, G, T/U, and C) of the oligonucleotide is an analog or ester of a naturally occurring nucleotide.

Modulate: The term modulate, as used herein, means to alter the expression level of a peptide, protein or polypeptide by increasing or decreasing its expression level relative to a reference expression level, and/or alter the stability and/or activity of a peptide, protein or polypeptide by increasing or decreasing its stability and/or activity level relative to a reference stability and/or activity level.

Non-covalent Bond: Non-covalent bond or non-covalent interaction refers to a chemical bond or interaction that does not involve the sharing of pairs of electrons between atoms. Examples of non-covalent bonds or interactions includes hydrogen bonds, ionic bonds (electrostatic bonds), van der Waals forces and hydrophobic interactions.

Nucleic Acid: Nucleic acid, as used herein, refers to any nucleic acid sequence containing DNA, RNA and/or analogs thereof and may include single, double and multi-stranded forms. The terms “nucleic acid”, “oligo”, “oligonucleotide” and “polynucleotide” may be used interchangeably.

Pharmaceutically Acceptable: Pharmaceutically acceptable, as used herein, means approved by a regulatory agency of a federal or a state government or listed in the U.S.

Pharmacopoeia or other generally recognized pharmacopoeia for use in animals and, more particularly, in humans.

Pharmaceutically Acceptable Salt: Pharmaceutically acceptable salt or salt of a compound (e.g., aptamer), as used herein, refers to a product that contains an ionic bond and is typically produced by reacting the compound with either an acid or a base, suitable for administering to an individual. A pharmaceutically acceptable salt can include, but is not limited to, acid addition salts including hydrochlorides, hydrobromides, phosphates, sulphates, hydrogen sulphates, alkyl sulphonates, arylsulphonates, arylalkylsulfonates, acetates, benzoates, citrates, maleates, fumarates, succinates, lactates, and tartrates; alkali metal cations such as Li, Na, K, alkali earth metal salts such as Mg or Ca, or organic amine salts.

Pharmaceutical Composition: Pharmaceutical composition, as used herein, refers to formulation comprising a pharmaceutical agent (e.g., drug) in a form suitable for administration to an individual. A pharmaceutical composition is typically formulated to be compatible with its intended route of administration. Examples of routes of administration include, but are not limited to, oral and parenteral, e.g., intravenous, intradermal, subcutaneous, inhalation, topical, transdermal, transmucosal, and rectal administration.

SELEX: The term SELEX, as used herein, refers to generally to the selection for nucleic acids that interact with a target molecule in a desirable manner, for example binding with high affinity to a protein; and the amplification of those selected nucleic acids. SELEX may be used to identify aptamers with high affinity to a specific target molecule. The term SELEX and “SELEX process” may be used interchangeably.

Sequence Identity: Sequence identity, as used herein, in the context of two or more nucleic acid sequences is a function of the number of identical nucleotide positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley-Interscience (1987)). As used herein, when describing the percent identity of a nucleic acid, such as an aptamer, the sequence of which is at least, for example, about 95% identical to a reference nucleotide sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleotide sequence or anywhere between those terminal positions, interspersed either individually among nucleotides in the reference sequence or in one or more contiguous groups within the reference sequence.

SOMAmer: The term SOMAmer, as used herein, refers to an aptamer having improved off-rate characteristics. SOMAmers are alternatively referred to as Slow Off-Rate Modified Aptamers, and may be selected via the improved SELEX methods described in U.S. Publication No. 20090004667, entitled “Method for Generating Aptamers with Improved Off-Rates”, which is incorporated by reference in its entirety.

Spacer Sequence: Spacer sequence, as used herein, refers to any sequence comprised of small molecule(s) covalently bound to the 5′-end, 3′-end or both 5′ and 3′ ends of the nucleic acid sequence of an aptamer. Exemplary spacer sequences include, but are not limited to, polyethylene glycols, hydrocarbon chains, and other polymers or copolymers that provide a molecular covalent scaffold connecting the consensus regions while preserving aptamer binding activity. In certain aspects, the spacer sequence may be covalently attached to the aptamer through standard linkages such as the terminal 3′ or 5′ hydroxyl, 2′ carbon, or base modification such as the CS-position of pyrimidines, or C8 position of purines.

Target Molecule: Target molecule (or target), as used herein, refers to any compound or molecule upon which a nucleic acid can act in a desirable manner (e.g., binding of the target, catalytically changing the target, reacting with the target in a way that modifies or alters the target or the functional activity of the target, covalently attaching to the target (as in a suicide inhibitor), and facilitating the reaction between the target and another molecule). Non-limiting examples of a target molecule include a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc. Virtually any chemical or biological effector may be a suitable target. Molecules of any size can serve as targets. A target can also be modified in certain ways to enhance the likelihood or strength of an interaction between the target and the nucleic acid. A target may also include any minor variation of a particular compound or molecule, such as, in the case of a protein, for example, variations in its amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean±20% of the indicated range, value, or structure, unless otherwise indicated. As used herein, the terms “include” and “comprise” are open ended and are used synonymously. It should be understood that the terms “a” and “an” as used herein refer to “one or more” of the enumerated components. The use of the alternative (e.g., “or”) should be understood to mean either one, both, or any combination thereof of the alternatives

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Detection Methods

Embodiments of the present disclosure provide methods for detecting protein levels in biological samples. The present disclosure is illustrated with aptamer detection technology. However, the present disclosure is not limited to aptamer detection technology. Any suitable detection method (e.g., immunoassay, mass spectrometry, histological or cytological methods, etc.) is suitable for use herein.

In some embodiments, aptamer based assays involve the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker level corresponding to a biomarker.

Aptamers for use in the disclosure may include up to about 100 nucleotides, up to about 95 nucleotides, up to about 90 nucleotides, up to about 85 nucleotides, up to about 80 nucleotides, up to about 75 nucleotides, up to about 70 nucleotides, up to about 65 nucleotides, up to about 60 nucleotides, up to about 55 nucleotides, up to about 50 nucleotides, up to about 45 nucleotides, up to about 40 nucleotides, up to about 35 nucleotides, up to about 30 nucleotides, up to about 25 nucleotides, and up to about 20 nucleotides.

In another aspect of this disclosure, the aptamer has a dissociation constant (K_{d}) for its target of about 10 nM or less, about 15 nM or less, about 20 nM or less, about 25 nM or less, about 30 nM or less, about 35 nM or less, about 40 nM or less, about 45 nM or less, about 50 nM or less, or in a range of about 3-10 nM (or 3, 4, 5, 6, 7, 8, 9 or 10 nM.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication No. 2009/0098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Publication No. US 2009/0004667, entitled “Method for Generating Aptamers with Improved Off-Rates”, which describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance. In some embodiments, an aptamer comprises at least one nucleotide with a modification, such as a base modification. In some embodiments, an aptamer comprises at least one nucleotide with a hydrophobic modification, such as a hydrophobic base modification, allowing for hydrophobic contacts with a target protein. Such hydrophobic contacts, in some embodiments, contribute to greater affinity and/or slower off-rate binding by the aptamer.

In some embodiments, an aptamer comprises at least two, at least three, at least four, at least five, at least six, at least seven, at least eight, at least nine, or at least 10 nucleotides with hydrophobic modifications, where each hydrophobic modification may be the same or different from the others.

In some embodiments, a slow off-rate aptamer (including an aptamers comprising at least one nucleotide with a hydrophobic modification) has an off-rate (t_{1/2}) of ≥30 minutes, ≥60 minutes, ≥90 minutes, ≥120 minutes, ≥150 minutes, ≥180 minutes, ≥210 minutes, or ≥240 minutes.

In some embodiments, an assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with a sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker level corresponding to a biomarker in the sample.

In some assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays or “aptamer based assay(s)” that permit an aptamer to capture its target in solution and then employ separation steps that are designed to remove specific components of the aptamer-target mixture prior to detection have also been described (see U.S. Publication No. 2009/0042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e., the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. In some embodiments of the methods described herein, the molecular capture reagents comprise an aptamer or an antibody or the like and the specific target may be a biomarker shown in Example 1.

In some embodiments, a method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker level in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex. For example, protein concentration or levels in a sample may be expressed as relative fluorescence units (RFU), which may be a product of detecting the aptamer component of the aptamer affinity complex (e.g., aptamer complexed to target protein create the aptamer affinity complex). That is, for an aptamer-based assay, the protein concentration or level correlates with the RFU.

A nonlimiting exemplary method of detecting biomarkers in a biological sample using aptamers is described in Kraemer et al., PLoS One 6(10): e26332.

Aptamers may contain modified nucleotides that improve it properties and characteristics. Non-limiting examples of such improvements include, in vivo stability, stability against degradation, binding affinity for its target, and/or improved delivery characteristics.

Examples of such modifications include chemical substitutions at the ribose and/or phosphate and/or base positions of a nucleotide. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH₂), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

Specific examples of a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) as illustrated immediately below.

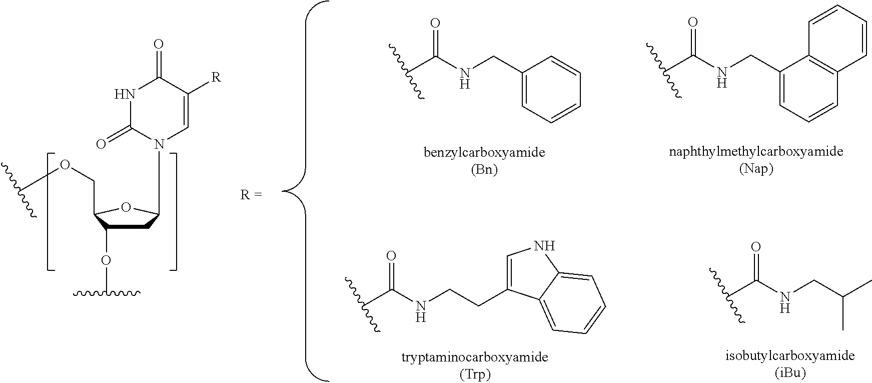

Chemical modifications of a C-5 modified pyrimidine can also be combined with, singly or in any combination, 2′-position sugar modifications, modifications at exocyclic amines, and substitution of 4-thiouridine and the like.

Representative C-5 modified pyrimidines include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium) propyl] carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine or 5-(N-[1-(2,3-dihydroxypropyl)]carboxyamide)-2′-deoxyuridine).

If present, a modification to the nucleotide structure can be imparted before or after assembly of the polynucleotide. A sequence of nucleotides can be interrupted by non-nucleotide components. A polynucleotide can be further modified after polymerization, such as by conjugation with a labeling component.

Additional non-limiting examples of modified nucleotides (e.g., C-5 modified pyrimidine) that may be incorporated into the nucleic acid sequences of the present disclosure include the following:

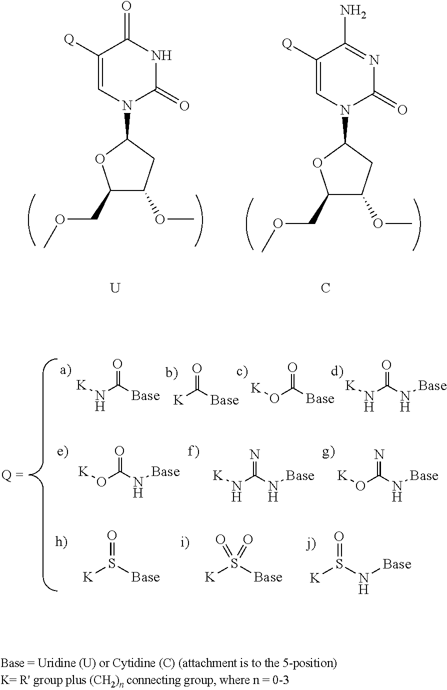

R′ is defined as follows:

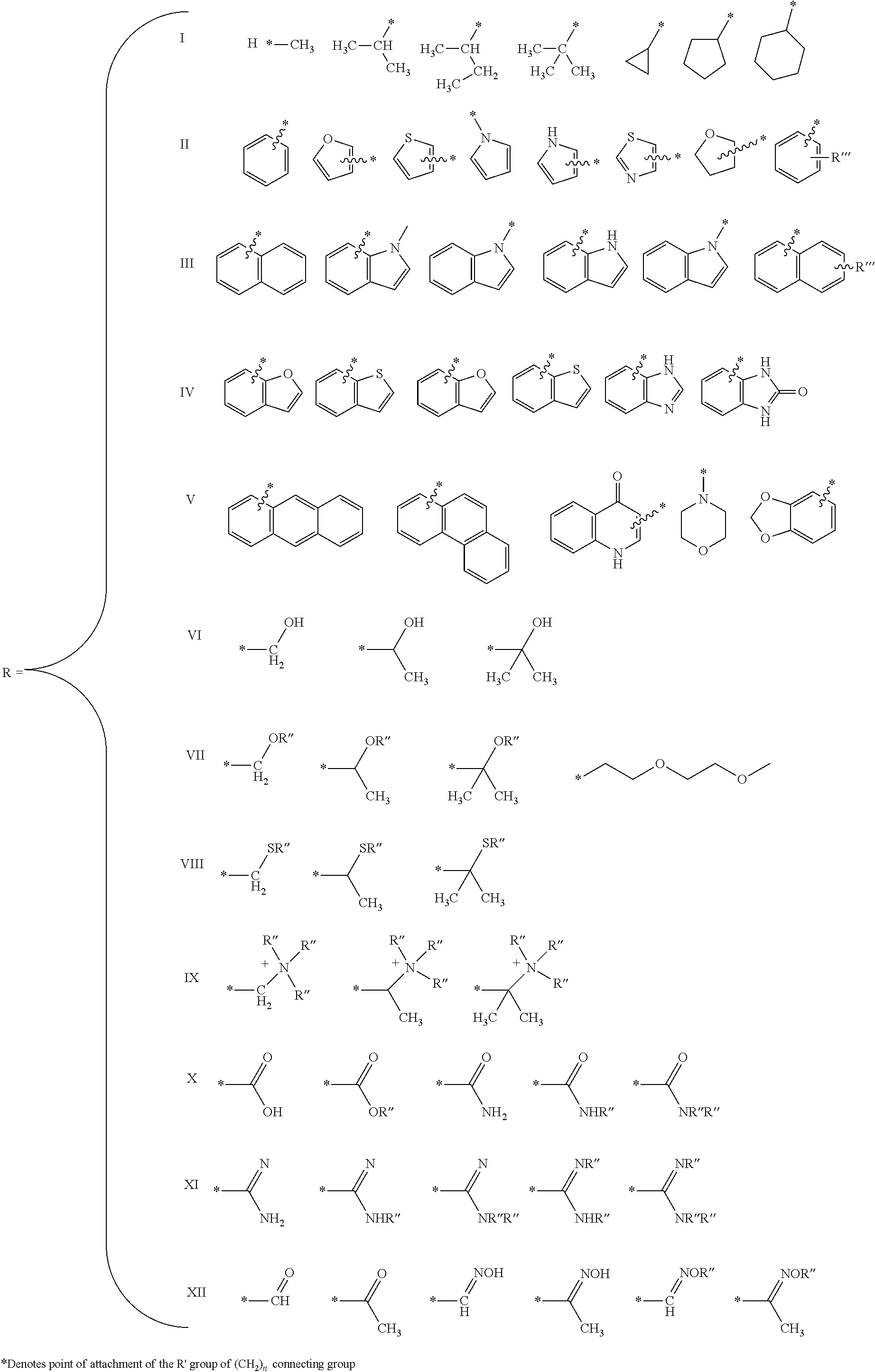

And, R″, R′″ and R″″ are defined as follows:
wherein
R″″ is selected from the group consisting of a. branched or linear lower alkyl (C1-C20); halogen (F, Cl, Br, I); nitrile (CN); boronic acid (BO₂H₂); carboxylic acid (COOH); carboxylic acid ester (COOR″); primary amide (CONH₂); secondary amide (CONHR″); tertiary amide (CONR″R′″); sulfonamide (SO₂NH₂); N-alkylsulfonamide (SONHR″).
wherein
R″, R′″ are independently selected from a group consisting of a branched or linear lower alkyl (C1-C2)); phenyl (C₆H₅); an R″″ substituted phenyl ring (R″″C₆H₄); wherein R″″ is defined above; a carboxylic acid (COOH); a carboxylic acid ester (COOR′″″); wherein R′″″ is a branched or linear lower alkyl (C1-C20); and cycloalkyl; wherein R″═R′″═(CH₂)ₙ; wherein n=2-10.

Further, C-5 modified pyrimidine nucleotides include the following:

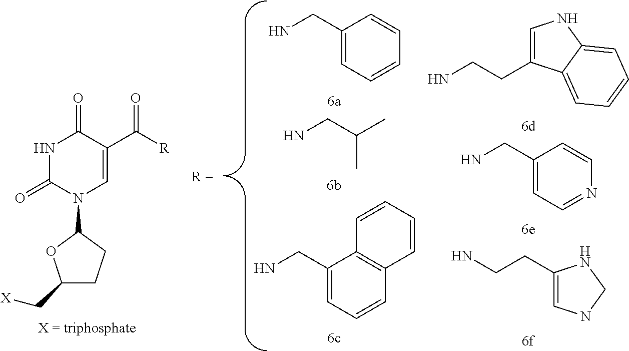

In some embodiments, the modified nucleotide confers nuclease resistance to the oligonucleotide. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

Polynucleotides can also contain analogous forms of ribose or deoxyribose sugars that are generally known in the art, including 2′-0-methyl-, 2′-0-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

The following examples are provided to illustrate certain particular features and/or embodiments. These examples should not be construed to limit the disclosure to the particular features or embodiments described.

The present disclosure provides kits comprising aptamers described herein. Such kits can comprise, for example, (1) at least one aptamer for identification of a protein target; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial or similar apparatus for holding and/or mixing the kit components; and (3) delivery apparatus.

### III. Personalized Therapeutic and Research Uses

In some embodiments, the present disclosure provides systems and methods for identifying proteins with altered expression in subjects with disease relative to subjects that do not have the disease. In some embodiments, proteins with altered expression serve as targets for drug screening and therapeutic applications. For example, in some embodiments, customized treatment is provided that is individualized to the proteomic profile of an individual subject's disease.

In some embodiments, proteins with altered expression are identified as targets for drug discovery. In some embodiments, proteins with existing drugs that target them are identified and such drugs are administered (alone or in combination with other drugs) to a subject. Thus, in some embodiments, the present disclosure provides customized treatment for a disease or condition.

In some embodiments, protein expression is compared to a reference sample from a disease-free subject or population of subjects. In some embodiments, the reference sample is sample of normal tissue from the subject, or a population average of normal tissue. In some embodiments, the level of the proteins is altered at least 2-fold (e.g., at least 4-fold, at least 5-fold, at least 10-fold, at least 20-fold, at least 50-fold, at least 100-fold, or more).

The present disclosure is suitable for identification of altered protein expression (e.g., using the assays described herein) in a variety of sample types. Examples include, but are not limited to, tissue, whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract, or cerebrospinal fluid.

The present disclosure is not limited to the identification of targets for a particular disease. In some embodiments, the disease is, for example, a cancer, a neoplasm, a tumor, and/or a metastatic form therein, a metabolic disorder, an inflammatory disease, or an infectious disease. In some embodiments, the cancer, neoplasm, tumor, or metastatic form therein is, for example, leukemia, lymphoma, prostate cancer, lung cancer, breast cancer, liver cancer, colorectal cancer, or kidney cancer. In some embodiments, the disease is lung cancer and the drug targets are one or more of AGER, THBS2, CA3, MMP12, PIGR, DCN, PGAM1, CD36, FABP, ACP5, CCDC80, PPBP, LYVE1, STC1, SPON1, IL17RC, MMP1, CA1, SERPINC1, TPSB2, CKB/CKBM, NAMPT/PBEF, PPBP/CTAPIII, F9, DCTPP1, F5, SPOCK2, CAT, PF4, MDK, BGN, CKM, POSTN, PGLYRP1, or CXCL12. In some embodiments, the drug targets and drugs are those shown in Tables 6 and 7.

In some embodiments, a computer-based analysis program is used to translate the raw data generated by the detection assay (e.g., the presence, absence, or amount of a given marker or markers) into data of value for a clinician (e.g., drug targets or drug(s) selection). The clinician can access the data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or other sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., protein expression data), specific for the diagnostic, therapeutic, or prognostic information desired for the subject.

The profile data is then prepared in a format suitable for interpretation by a treating clinician. For example, rather than providing raw expression data, the prepared format may represent a suggested treatment course of action (e.g., specific drugs for administration). The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a treatment outcome or for drug discovery.

Some exemplary biomarkers and drugs that target the altered expression of the biomarker are described herein (See e.g., WO 2010/0028288; herein incorporated by reference in its entirety. The markers and drugs described herein are not limiting. Additional markers and drugs are specifically contemplated.

For example, in some embodiment, c-kit (also known as CD117, KIT, PBT, SCFR), Bcr-Abl fusion, platelet derived growth factor receptor (PDGFR), are targeted with imatinib mesylate (Gleevec); PDGFR is targeted with Sutent (Sunitib or SUI 1248), a receptor tyrosine kinase inhibitor; secreted protein acidic and rich in cysteine (SPARC; also known as ON, osteonectin) is targeted with Abraxane; HSP90 (also known as HSPN; LAP2; HSP86; HSPC1; HSPCA; Hsp89; HSP89A; HSP90A; HSP90N; HSPCAL1; HSPCAL4; FLB1884; HSP90AA1) is targeted with CNF2024 (BIIB021); MGMT (0-6-methylguanine-DNA methyltransferase) is targeted with temozolomide (Temodar, Temodal); HER2 (also known as ERBB2, NED, NGL, TKR1, CD340, HER-2, HER-2/neu) is targeted with trastuzumab (Herceptin); human epidermal growth factor receptor 1 (also known as HER1, EGFR, ERBB, mENA, ERBB1, PIG61) is targeted with Erlotinib (Tarceva), gefitinib, panitumumab (Vectibix), lapatinib, or cetuximab (Erbitux); vascular endothelial growth factor (VEGF) is targeted with Bevacizumab (Avastin); ER (also known as estrogen receptor; ESR; Era; ESRA; NR3A1; DKFZp686N23 123; ESR1) is targeted with hormonal therapeutics (e.g., ER blockers such as tamoxifen, or aromatase inhibitors, such as anastrozole); PR (also known as progesterone receptor; NR3C3; PGR) is targeted with is targeted with hormonal therapeutics (e.g., ER blockers such as tamoxifen, or aromatase inhibitors, such as anastrozole); vras and Kras are targeted with bevacizumab (Avastin); TOPO1 (also known as DNA topoisomerase; TOPI; TOP1) is targeted with fluorouracil (5-FU; FSU; Adrucil) with or without irinotecan or oxaliplatin; Phosphatase and Tensin Homolog (PTEN) is targeted with cetuximab (Erbitux) or panitumumab (Vectibix); PIK3CA is targeted with cetuximab (Erbitux) or panitumumab (Vectibix); Kras (also known as v-Ki-ras2 Kirsten rat sarcoma viral oncogene homolog; NS3; KRAS1; KRAS2; RASK2; KI-RAS; C-K-RAS; K-RAS2A; K-RAS2B; K-RAS4A; K-RAS4B) is targeted with bevacizumab (Avastin), cetuximab (Erbitux), erlotinib (Tarceva), gefitinib (Iressa), or panitumumab (Vectibix); Nrf2 (also known as nuclear factor (erythroid-derived 2)-like 2; NFE2L2) is targeted with doxorubicin (Adriamycin); DPD (also known as dihydropyrimidine dehydrogenase; DHP; DHPDHASE; MGC70799; MGC132008; DPYD) is targeted with fluorouracil (5-FU); OPRT (also known as uridine monophosphate synthetase; LIMPS uridine monophosphate synthase; OPRtase; OMPdecase; UMP synthase; orotidine 5′-phosphate decarboxylase; orotate phosphoribosyltransferase phosphoribosyltransferase; orotate phosphoribosyl transferase; orotidine-5′decarboxylase) is targeted with 5-FU; TS (also known as thymidylate synthetase; TMS; TSase; HsT422; MGC88736; TYMS) is targeted with 5-FU; BRAF is targeted with cetuximab (Erbitux) or panitumumab (Vectibix); thymidylate synthase is targeted with 5-FU; or those described in Tables 6 or 7.

The present disclosure further provides for a method for identifying one or more patient subpopulations from a plurality of patients diagnosed with the same disease or condition, the method comprising: detecting the level of one or more proteins in a biological sample from each patient of the plurality of patients; comparing the level of the one or more proteins from each patient within the plurality of patients, and identifying one or more patient subpopulations, wherein each patient subpopulation of the one or more patient subpopulations is distinguished from another patient subpopulation based on the difference in the level of the one or more proteins, and wherein the difference in the level of the one or more proteins is selected from the group consisting of at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100) and at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

The present disclosure further provides for a method for selecting one or more drugs to treat a subject having a disease or condition, the method comprising: acquiring knowledge of the level of one or more proteins in a biological sample from the subject, wherein at least one of the one or more proteins is a drug target; and selecting one or more drugs to treat the subject based on the level of the one or more proteins, wherein at least one drug of the one or more drugs is a drug to at least one of the one or more proteins.

In another aspect, the selecting one or more drugs to treat the subject is based on the difference in the level of the one or more proteins from the subject compared to the level of the respective one or more proteins from a reference biological sample, subject or population, and wherein the difference is at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 fold).

In another aspect, the selecting one or more drugs to treat the subject is based on the difference in the level of the one or more proteins from the subject compared to the level of the respective one or more proteins from a reference biological sample, subject or population, and wherein the difference is at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

In another aspect, the method further comprises administering the one or more drugs to the subject, thereby treating the disease or condition in the subject.

In another aspect, the method further comprises selecting the one or more drugs to treat the subject based on acquiring knowledge of one or more complete or partial gene sequences of the subject.

In another aspect, the method further comprises selecting the one or more drugs to treat the subject based on acquiring knowledge of one or more genetic mutations from the subject.

In another aspect, the disease or condition is selected from the group consisting of a cancer, a metabolic disorder, an inflammatory disease and an infectious disease.

In another aspect, the biological sample is selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid.

The present disclosure further provides for method for selecting one or more drugs to treat a subject having a disease or condition, the method comprising: detecting the level of one or more proteins in a biological sample from the subject, wherein, at least one of the one or more proteins is a drug target; and selecting one or more drugs to treat the subject based on the level of the one or more proteins, wherein at least one drug of the one or more drugs is a drug to at least one of the one or more proteins.

In another aspect, the selecting one or more drugs to treat the subject is based on the difference in the level of the one or more proteins from the subject compared to the level of the respective one or more proteins from a reference biological sample, subject or population, wherein the difference is at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 fold). In another aspect, the selecting one or more drugs to treat the subject is based on the difference in the level of the one or more proteins from the subject compared to the level of the respective one or more proteins from a reference biological sample, subject or population, and wherein the difference is at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

In another aspect, the method further comprises administering the one or more drugs to the subject, thereby treating the disease or condition in the subject.

In another aspect, the method further comprises selecting the one or more drugs to treat the subject based on acquiring knowledge of one or more complete or partial gene sequences of the subject.

In another aspect, the method further comprises selecting the one or more drugs to treat the subject based on acquiring knowledge of one or more genetic mutations from the subject. In another aspect, the disease or condition is selected from the group consisting of a cancer, a metabolic disorder, an inflammatory disease and an infectious disease.

In another aspect, the biological sample is selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid.

In another aspect, the detecting the level of one or more proteins in a biological samples is performed by an assay selected from the group consisting of an aptamer-based assay, an antibody based assay and a mass spectrometry assay.

The present disclosure further provides for a treatment plan for a subject having a disease or condition comprising: one or more drugs, wherein the selection of the one or more drugs is based on the level of one or more proteins, wherein at least one of the one or more proteins is a drug target, and wherein at least one drug of the one or more drugs is a drug to at least one of the one or more proteins; and administering the one or more drugs to the subject, thereby treating the disease or condition in the subject.

In another aspect, the selecting one or more drugs to treat the subject is based on the difference in the level of the one or more proteins from the subject compared to the level of the respective one or more proteins from a reference biological sample, subject or population, wherein the difference is at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 fold). In another aspect, the selecting one or more drugs to treat the subject is based on the difference in the level of the one or more proteins from the subject compared to the level of the respective one or more proteins from a reference biological sample, subject or population, and wherein the difference is at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

In another aspect, the method further comprises administering the one or more drugs to the subject, thereby treating the disease or condition in the subject.

In another aspect, the method further comprises selecting the one or more drugs to treat the subject based on acquiring knowledge of one or more complete or partial gene sequences of the subject.

In another aspect, the method further comprises selecting the one or more drugs to treat the subject based on acquiring knowledge of one or more genetic mutations from the subject.

In another aspect, the disease or condition is selected from the group consisting of a cancer, a metabolic disorder, an inflammatory disease and an infectious disease.

In another aspect, the biological sample is selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, cytologic fluid, nipple aspirate, bronchial aspirate, bronchial brushing, synovial fluid, joint aspirate, organ secretions, cells, a cellular extract and cerebrospinal fluid. In another aspect, the detecting the level of one or more proteins in a biological samples is performed by an assay selected from the group consisting of an aptamer-based assay, an antibody based assay and a mass spectrometry assay.

In another aspect, the one or more drugs is selected from the group consisting of 4-Aminosalicylic_acid, Abatacept, Abciximab, Acetaminophen, Acetazolamide, Acetohydroxamic_acid, Adalimumab, Adenine, Adenosine_monophosphate, Adenosine_triphosphate, Afatinib, Aflibercept, Alclometasone, Aldesleukin, Alefacept, Alemtuzumab, Aliskiren, Alpha_1-antitrypsin, Alteplase, Aluminium, Amcinonide, Amiloride Aminocaproic_acid, Aminophylline, Amitriptyline, Amlodipine, Amrinone, Anagrelide, Anakinra, Anistreplase, Antihemophilic_Factor, Antrafenine, Apixaban, Aprotinin, Ardeparin, Argatroban, Arsenic_trioxide, Aspirin, Atorvastatin, Auranofin, Avanafil, Axitinib, Bacitracin Balsalazide, Basiliximab, Becaplermin, Beclometasone_dipropionate, Belatacept, Belimumab, Bendroflumethiazide, Betamethasone, Bevacizumab, Bivalirudin, Bosutinib, Brentuximab_vedotin, Brinzolamide, Bromfenac, Budesonide, Cabozantinib, Canakinumab, Capecitabine, Capromab, Captopril, Carbidopa, Carbimazole, Carprofen, Carvedilol, Cefazolin, Cefdinir, Celecoxib, Certolizumab_pegol, Cetuximab, Chloramphenicol, Chloroquine, Chlorothiazide, Chlorotrianisene, Ciclesonide, Cilostazol, Clenbuterol, Clobetasol_propionate, Clocortolone, Clomifene, Clomipramine, Cortisone_acetate, Creatine, Cyclosporine, Cysteamine, Dabigatran, Dacarbazine, Daclizumab, Dalteparin_sodium, Danazol, Darbepoetin_alfa, Dasatinib, Denileukin_diftitox, Denosumab, Desogestrel, Desonide, Desoximetasone, Dexamethasone, Dextrothyroxine, Diazoxide, Dichlorphenamide, Diclofenac, Dienestrol, Diethylstilbestrol, Diflorasone, Diflunisal, Difluprednate, Dipyridamole, Docetaxel, Dorzolamide, Drotrecogin_alfa, Eculizumab, Efalizumab, Eicosapentaenoic_acid, Eltrombopag, Enoxaparin, Enoximone, Epoetin alfa, Eptifibatide, Equilin, Erlotinib, Erythropoietin, Estradiol Estramustine, Estriol, Estrone, Estropipate, Etanercept, Ethinamate, Ethinylestradiol, Ethoxzolamide, Ethynodiol diacetate, Etodolac, Etonogestrel, Etoricoxib, Factor_IX, Factor_VII, Fenoprofen, Filgrastim, Floxuridine, Fludrocortisone, Fludroxycortide, Flunisolide, Fluocinolone_acetonide, Fluocinonide, Fluorometholone, Fluorouracil, Fluoxymesterone, Flurbiprofen, Fluticasone_furoate, Fluticasone_propionate, Fluvastatin, Fomepizole, Fondaparinux_sodium, Fulvestrant, Furosemide, Gadopentetate_dimeglumine, Gefitinib, Gemcitabine, Gemtuzumab_ozogamicin, Ginkgo_biloba, Ginseng, Gliclazide, Glucosamine, Glutathione, Golimumab, Heparin, Hyaluronidase, Hydrochlorothiazide, Hydrocortisone, Hydroxocobalamin, Ibritumomab, Ibudilast, Ibuprofen, Iloprost, Imatinib, Indomethacin, Infliximab, Ingenol_mebutate, Inhaled_insulin, Insulin, Insulin_aspart, Insulin_detemir, Insulin_glargine, Insulin_glulisine, Insulin_lispro, Interferon_gamma-1b, Ipilimumab, Irinotecan, Isoproterenol, Ketoprofen, Ketorolac, Ketotifen, Lapatinib, L-Aspartic_Acid, L-Carnitine, L-Cysteine, Lenalidomide, Lepirudin, Leucovorin, Levonorgestrel, Levosimendan, Lidocaine, Lisinopril, Lithium, L-Leucine, Loperamide, Lornoxicam, Loteprednol, Lovastatin, L-Proline, Lucanthone, Lumiracoxib, Magnesium salicylate, Marimastat, Meclofenamic_acid, Medroxyprogesterone, Medrysone, Mefenamic_acid, Megestrol, Melatonin, Meloxicam, Menadione, Mesalazine, Mestranol, Metformin, Methazolamide, Methimazole, Methocarbamol, Methyl_aminolevulinate, Methylprednisolone, Mifepristone, Milrinone, Mimosine, Minocycline,

Moexipril, Mometasone, Muromonab, Mycophenolate_mofetil, Mycophenolic_acid, Nabumetone, Naloxone, Naproxen, Natalizumab, Nedocromil, Nepafenac, Nilotinib, Nitroxoline, Norgestimate, NPH_insulin, Ocriplasmin, Olsalazine, Oprelvekin, Ornithine, Ospemifene, Oxaprozin, Oxtriphylline, Paclitaxel, Palifermin, Paliperidone, Palivizumab, Panitumumab, Paramethasone, Pazopanib, Pegaptanib, Pegfilgrastim, Peginesatide, Pemetrexed, Pentoxifylline, Pertuzumab, Phenazone, Phenelzine, Phenformin, Phenylbutazone, Phosphatidylserine, Piroxicam, Pitavastatin, Pomalidomide, Ponatinib, Porfimer, Pralatrexate, Pranlukast, Pravastatin, Prednicarbate, Prednisolone, Prednisone, Proflavine, Progesterone, Propylthiouracil, Pyruvic_acid, Quinestrol, Quinethazone, Raloxifene, Raltitrexed, Ranibizumab,
Rasagiline, Regorafenib, Remikiren, Reteplase, Ribavirin, Rifabutin, Rilonacept, Rimexolone, Rituximab, Rivaroxaban, Roflumilast, Romiplostim, Rosuvastatin, Ruxolitinib, Salicyclic_acid, Sargramostim, Sildenafil, Simvastatin, Sirolimus, Sodium hyaluronate, Sodium_salicylate, Sodium_stibogluconate, Somatropin_recombinant, Sorafenib, Streptokinase, Sucralfate, Sulfasalazine, Sulindac, Sulodexide, Sunitinib, Suprofen, Suramin, Tadalafil, Tamoxifen, Tenecteplase, Thalidomide, Theophylline, Tiaprofenic_acid, Tiludronate, Tirofiban, Tocilizumab, Tofacitinib, Tofisopam, Tolmetin, Topiramate, Topotecan, Toremifene, Tositumomab, Trametinib, Tranexamic_acid, Trastuzumab, Trastuzumab_emtansine, Triamcinolone, Trifluridine, Trilostane, Trimethoprim, Udenafil, Urokinase, Vandetanib, Vardenafil, Vitamin_E, Vorinostat, WF10, Ximelagatran, Zonisamide and a combination thereof.

The present disclosure further provides for a method for identifying a drug target, the method comprising: acquiring knowledge of the level of one or more proteins in a biological sample from a subject; and selecting at least one of the one or more proteins as a target for drug development; wherein, the at least one of the one or more proteins selected as a target is selected based on the difference in the level of the at least one of the one or more proteins from the biological sample from the subject compared to the level of the respective at least one of the one or more proteins from a reference biological sample, subject or population, and wherein the difference in the level of the one or more proteins is selected from the group consisting of at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100) and at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

In another aspect, the at least one of the one or more proteins selected as a target for drug development is not a drug target.

The present disclosure further provides for a method for identifying a drug target, the method comprising: detecting the level of one or more proteins in a biological sample from a subject; and selecting at least one of the one or more proteins as a target for drug development;

wherein, the at least one of the one or more proteins selected as a target is selected based on the difference in the level of the at least one of the one or more proteins from the biological sample from the subject compared to the level of the respective at least one of the one or more proteins from a reference biological sample, subject or population, and wherein the difference in the level of the one or more proteins is selected from the group consisting of at least from 2-fold to 100-fold (or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100) and at least from 0.5-fold to 0.01-fold (or 0.5, 0.4, 0.3, 0.2, 0.1, 0.09, 0.08, 0.07, 0.06, 0.05, 0.04, 0.03, 0.02 or 0.01 fold).

### EXAMPLES

### Example 1

Materials and Methods

### Tumor Specimens

Lung cancer tumor tissue and matched non-tumor tissue were harvested at the time of surgical resection and stored frozen in the Colorado SPORE in Lung Cancer Tissue Bank. Pathological inspection was performed on 29 of the tumor samples to determine the proportion of the tissue that contained inflammation, necrosis or stroma. The average and interquartile (IQR) range for these parameters were: inflammation 16% (IQR 5-20%), necrosis 10% (IQR 0-15%), and stroma 31% (IQR 20-40%).

### Proteomic Sample Preparation and Tumor Mutation Detection

Protein lysates were prepared from 63 tumor and matched non-tumor tissue as described (Mehan 2012). Multiplexed single nucleotide extension sequencing (SNaPshot, Life Technologies), which involves multiplexed PCR, mutiplexed single-base primer extension, and capillary electrophoresis, was performed on 49 of the tumors (Doebele 2012, Su 2011). The mutations detected by the SNaPshot panel are listed in table 1.

TABLE 1
SNaPshot Multiplex Mutation Panel

| | |
|---|---|
| AKT1 | E17K |
| APC | R1114*, Q1338*, R1450*, T1556NinsA |
| CTNNB1 | D32Y, D32H, D32N, D32V, D32G, S33Y, S33F, S33C, |
| | G34V, G34E, S37P, S37A, S37F, S37Y, S37C, T41A, |
| | T41S, T41P, T41N, T41I, S45P, S45A, S45F, S45Y, S45C |
| BRAF | G466V, G469A, L597V, V600E, V600M |
| KIT | D816V |
| EGFR | G719S, G719C, G719A, del_746-750, T790M, |
| | L858R, L861Q |
| FLT3 | D835Y |
| JAK2 | V617F |
| KRAS | G12R, G12S, G12D, G12V, G12A, G13D, G13A, G13S, |
| | G13R, G13C, Q61H, Q61K, G61L, Q61R |
| MAP2K1 | Q56P, K57N, D67N |
| NRAS | G12S, G12R, G12C, G12D, G12A, G12V, G13S, G13R, |
| | G13C, G13D, G13A, G13V, Q61K, Q61L, Q61R |
| NOTCH1 | L1575Q, L1575P, H1601 |
| PIK3CA | R88Q, Q546K, Q546E, Q546R, Q546P, H1047R, |
| | H1047Y, G1049S |
| PTEN | R130G, R130*, R173C, R233*, K297fs |
| TP53 | R175L, R175H, G245S, G245R, G245C, R248W, R248G, |
| | R248Q, R248P, R248L, R273C, R273L, R273H, R306* |

### Proteomic Analysis

Tissue lysates (2 ug total protein/sample) were analyzed with the SOMAscan V3 proteomic assay, which measures 1,129 proteins (Gold 2010). The SOMAscan analytes cover a broad range of proteins associated with disease physiology and biological functions, including cytokines, kinases, growth factors, proteases and their inhibitors, receptors, hormones and structural proteins (Mehan 2013). SOMAscan uses novel modified DNA aptamers called SOMAmers to specifically bind protein targets in biologic samples (Gold 2010, Vaught 2010). All sample analyses were conducted in a Good Laboratory Practice (GLP) compliant lab at Somalogic as described (Kraemer 2011). The samples were distributed randomly in the assay and the assay operators were blinded to the identity of all samples. Microarray images were captured and processed with a microarray scanner and associated software. Each sample in the study was normalized by aligning the median of each sample to a common reference. Inter-plate and inter-run calibration was done by applying a multiplicative scaling coefficient to each SOMAmer.

### Statistical Analysis

### Data

All data were derived from the lung cancer tissue study known as the Lungevity study, CL-13-012. SOMAscan data for a number of paired samples consisting of tumor tissue or presumably normal adjacent tissue were obtained. Data were selected from the raw data file for further analysis as follows:
Some samples are duplicated and those values were averaged to produce the final data used for analysis.
Data from the file were limited to only those tumor samples labeled as ‘Adeno’ or ‘Squamous’ and their cognate normal sample.
Some cases exist where one or the other cognate pair is not present, and those unpaired samples were removed.
   The final data collection contained 63 paired samples. Paired sample data were converted to ratios by dividing the tumor sample RFU value by the control sample RFU value.

### Response Algorithm

A cutoff was defined to apply to the ratio data. Values were linked to the threshold value and change in sync with user changes. The number of samples found above or below, respectively, this threshold was calculated for each protein individually. The number of proteins found above or below, respectively, the threshold value for each sample was tabulated individually. The data table is sorted from left to right in decreasing order of the values tabulated. Effectively, this leads to an ordering of the proteins by the number of samples found outside the given threshold. The following data was then extracted:
The number of samples outside threshold (up or down) for each protein;
The number of proteins (up or down) outside threshold for each sample;
The newly ordered GeneName:SomamerID values;
Annotations for each GeneName:SomamerID (sp., full protein name, drug list, and pathway information);
The newly ordered table of ratio values.

Conditional formatting is programmatically applied to the ratio data table in order to illustrate those values which are over-expressed above the threshold or under-expressed below the threshold.

Demographic Tables are shown in Tables 2-4 below

TABLE 2
Tumor Histology and Stage

| | Histology | | |
|---|---|---|---|
| | Adeno | Squamous | |
| Stage | (n = 45) | (n = 18) | Total |
| I A | 21 | 6 | 27 |
| I B | 8 | 6 | 14 |
| II A | 7 | 2 | 9 |
| II B | 3 | 2 | 5 |
| III A | 6 | 2 | 8 |

TABLE 3
Mutations Identified*

| | Histology | | |
|---|---|---|---|
| Mutation | Adeno | Squamous | Total |
| APC | 1 | 0 | 1 |
| BRAF | 3 | 0 | 3 |
| EGFR | 5 | 0 | 5 |
| KRAS | 13 | 0 | 13 |
| PIK3CA | 0 | 2 | 2 |
| TP53 | 0 | 2 | 2 |
| None Found | 17 | 6 | 23 |
| No Data | 6 | 8 | 14 |

*Two tumors had both PIK3CA and KRAS mutations and one tumor had both KRAS and TP53 mutations.

TABLE 4
Patient Characteristics*

| Parameter | Count |
|---|---|
| Median Age (IQR) | 68.5 (61-76) |
| Gender | |
| Male | 34 (59%) |
| Female | 24 (41%) |
| Tobacco User | |
| Current | 12 (22%) |
| Former | 37 (67%) |
| Never | 6 (11%) |
| Median Pack Years (IQR) | 45 (27-62) |

### Results

1,170 proteins were measured in two samples (NSCLC, the tumors, and adjacent healthy lung tissue) from 63 people, for a total of 63×2×1,129=142,254 measurements. For small tumors, the entire tumor was sampled, while for larger tumors a piece was homogenized. In some experiments, larger tumors are subdivided into samples at whatever distances are possible. Unlike antibodies, SOMAmers are identified through a variant of the SELEX method and are made of modified DNA. SOMAmers recognize conformational epitopes on the target proteins. A few of the menu SOMAmers were identified with rodent proteins that are nearly identical to their human homologue. SOMAmers are analogous to the antigen-combining sites of antibodies, they are monovalent, and they bind with high affinity and dissociate slowly from their target proteins. Spike and recovery experiments have shown that in plasma, serum, and buffer, spikes lead to higher signals in the SOMAscan assay. Pull-downs in plasma or serum with the menu SOMAmer identified the target protein by both gels and Mass Spec as the intended analyte. SOMAscan yields data in fluorescent units, such that comparisons can be made between two tissues with ease (providing Relative Fluorescent Units—RFUs—that can be compared). Standard curves are used to convert RFUs to an approximate absolute protein when desired.

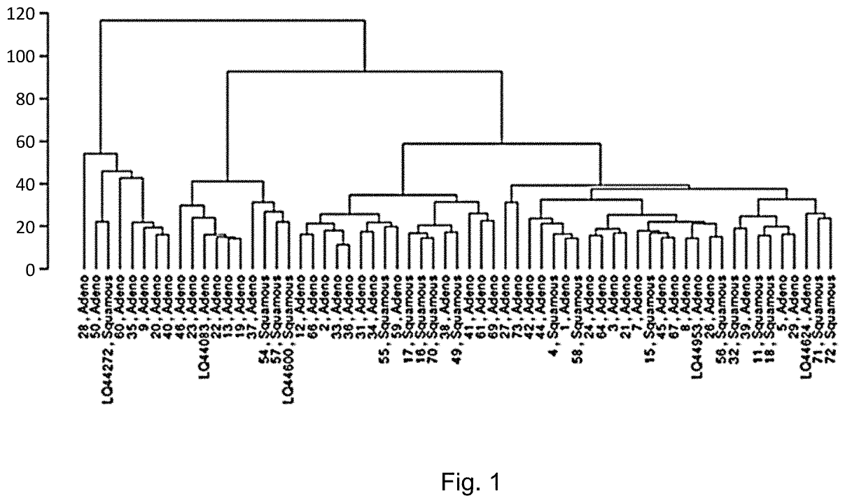

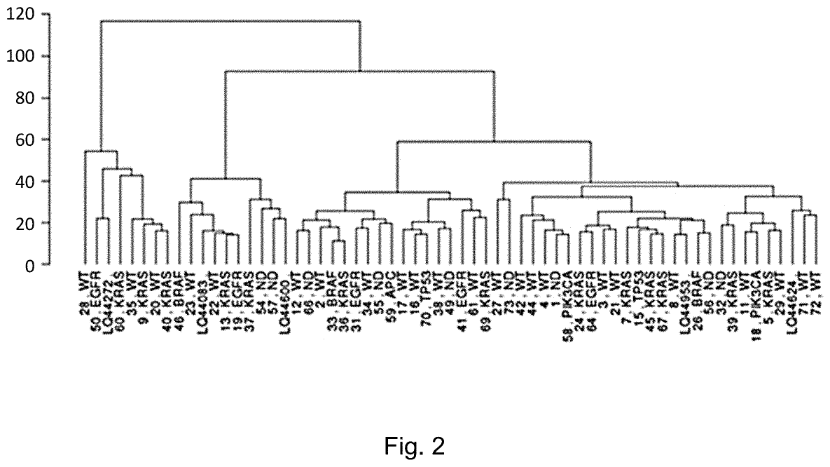

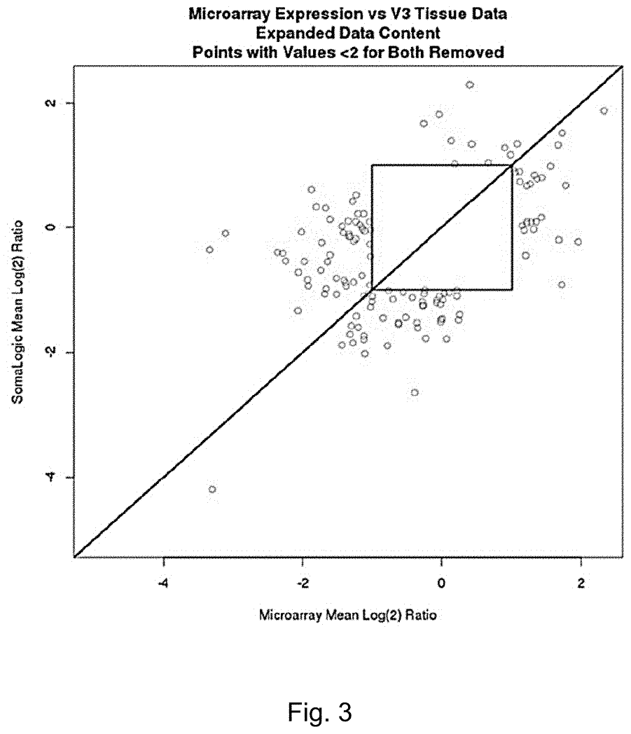

Relative protein levels that are more than 4-fold up or down in the tumors compared to the healthy tissue were selected; this level of change was selected in this study because an analyte that shows more than 4-fold up or down was not considered likely to represent a “false discovery.” However, the present invention is not so limited. For example, in other embodiments, a fold change (e.g., up or down) of less than 4-fold (e.g., 3-fold, 2-fold, or lower) or more than 4-fold (e.g., 5-fold, 10-fold, 100-fold, or higher) may be used. Of the 1,129 proteins measured for 63 pairs of tissues on SOMAscan, 2 proteins were up or down 4-fold or more for 51 pairs of samples (of the 63 pairs), 2 other proteins were up or down 4-fold or more for 40 pairs of samples, 4 other proteins were up or down 4-fold or more for 30 pairs of samples, 27 other proteins were up or down 4-fold or more for 20 pairs of samples, 81 other proteins were up or down 4-fold or more for 10 pairs of samples, and 415 other proteins were up or down 4-fold or more for fewer than 10 pairs (but for at least one pair). More than 600 proteins were not up or down 4-fold or more in any pair. These data are shown in FIG. 1.

A total of 35 proteins were up or down 4-fold or more in 20 pairs of tissue, with more proteins up or down in fewer sample pairs. The largest class of proteins was in no sample pair up or down 4-fold or more.

When the data was observed in heat maps of clusters to compare proteomics for mutations, pathology and stages, as well as clustering by the protein levels themselves, no obvious clusters emerge when forced by the standard definitions of NSCLC.

The Top 35 Proteins that Distinguish NSCLC from Healthy Lung Tissue

Of the 35 proteins which were the top biomarkers in the study (Table 5) (“top” equals the proteins that are different between tumors and healthy adjacent tissue by 4-fold or more in 20 pairs or more), two proteins distinguish between squamous cell carcinoma and adenocarcinoma. For the overwhelming majority of biomarkers, adenocarcinoma and squamous cell carcinoma appear to be very similar cancers.

No correlations were found between the mutations and the levels of these 35 proteins. Some tumors with the same pathology and the identical KRAS mutations—in one such tumor 190 proteins were over or under expressed by four-fold or more, and in another tumor with the same pathology and KRAS mutation only 3 proteins were four-fold more or less abundant.

TABLE 5

| Protein name | N/63 | Up or Down | Squamous/Adeno |
|---|---|---|---|
| AGER | 51 | Down | Same |
| THBS2 | 51 | Up | Same |
| CA3 | 45 | Down | Same |
| MMP12 | 41 | Up | Same |
| PIGR | 37 | Mixed | Different |
| DCN | 35 | Mixed | Same |
| PGAM1 | 32 | Up | Same |
| CD36 | 30 | Down | Same |
| FABP* | 29 | Down | Same |
| ACP5 | 29 | Down | Same |
| CCDC80 | 29 | Mixed | Same |
| PPBP | 28 | Down | Same |
| LYVE1 | 28 | Down | Same |
| STC1 | 28 | Up | Same |
| SPON1 | 27 | Down | Same |
| IL17RC | 26 | Down | Same |
| MMP1 | 26 | Up | Same |
| CA1 | 25 | Down | Same |
| SERPINC1 | 25 | Down | Same |
| TPSB2 | 25 | Down | Same |
| CKB/CKBM | 25 | Down | Same |
| NAMPT/PBEF | 25 | Up | Same |
| PPBP/CTAPIII | 23 | Down | Same |
| F9 | 23 | Down | Same |
| DCTPP1 | 23 | Up | Same |
| F5 | 23 | Down | Same |
| SPOCK2 | 23 | Down | Same |
| CAT | 21 | Down | Same |
| PF4 | 21 | Down | Same |
| MDK | 21 | Up | Same |
| BGN | 21 | Down | Same |
| CKM | 21 | Down | Same |
| POSTN | 20 | Up | Same |
| PGLYRP1 | 20 | Mixed | Different |
| CXCL12 | 20 | Down | Same |

Proteins that Distinguish NSCLC from Healthy Tissue

Further analysis was conducted on proteins that show different concentrations less frequently between tumor and healthy tissue. Differences between tumors and healthy adjacent tissues were neither correlated with pathology or genetics.

### Drug Interventions

Proteins that are elevated in individual tumors are targets for a drug (e.g., existing or new drug), whether that drug was developed for cancer or not. In some embodiments, existing drugs are utilized. In some embodiments, other proteins in the same pathways as targets identified herein are targeted.

Of the 1,129 proteins analyzed, 690 (61%) displayed at least a 4-fold difference with one or more of the paired samples. The 63 tumors displayed a continuum of the number of proteins, up or down 4-fold compared with healthy tissue, from 3 to 190.

Some of the drugs provided herein are already approved for cancer patients. Others are approved but not for cancer. Trials are designed to assess their value as individualized therapeutics. In other cases unapproved inhibitors are starting points for development of new drugs that are used for individually targeted tumors.

At the highest view of the data, both the similarities and diversities in tumor-specific expression protein concentrations were observed.

NSCLC's (and other cancer types) show common proteins that are both elevated and reduced in concentrations. These proteins are generally related to processes that drive most cancers: cell-autonomous growth rates and the ability to overcome contact inhibition, capacity to grow under limited oxygen levels as they exceed the local blood supply, defenses against immune and inflammatory surveillance, invasiveness and metastatic potential, and other processes (e.g., the capacity to utilize the lymphatic system as a source of nutrients when the blood supply is inhibited by angiogenesis intervention). Among the common proteins with elevated concentrations, proteins expected to be “ups” were not found—these expectations are summarized by the modes of actions of several cancer drugs, which turn out to not be useful, frequently, in large numbers of patients with NSCLC.

NSCLC's (and other cancer types) show elevated levels of rare proteins that allow the required cancer processes, both known and unknown. The data show that several tumors that differ in every possible way and seem to have no difficulties being a tumor by all extant definitions.

Thus, the present invention provides that, in some embodiments, the tumor proteome is independent of the pathology report and the mutations that may have caused the tumor and which may still be present—critically or not—in the tumor. The properties required for cancer growth and metastasis, are, in some embodiments, different than the properties (e.g., genes) utilized in the early stages of tumor formation. In some embodiments, the invention provides that the final proteomic state of a cancer is driven by selection in an individual and not by selection in a mouse or a petri dish; individuals present the personalized environment against which selection occurs.

Accordingly, in some embodiments, the present invention provides methods for physicians and patients to obtain SOMAscan analyses of their tumors relative to the healthy tissues from which the tumor was derived. Reports to the physicians and patients include every protein that is present at altered levels relative to controls and the pathway within which that protein is found, along with drugs that antagonize or agonize the protein or pathway of interest. In some embodiments, an elevated protein is a driver of the cancer, and a drug may be available that antagonizes the protein or pathway. In some embodiments, no drug may yet be approved that antagonizes that protein or pathway, but as clinical trial for such a therapeutic NSCLC may be available. In some embodiments, an approved drug may exist aimed at that protein for a different disease—another cancer or something completely different—and in that case the physician and the patient may discuss the advantages and disadvantages of such a treatment.

In some embodiments, a patient's tumor does not display properties or characteristics of protein or pathway that may respond to a standard treatment, but does display an increase of a protein in the tumor that would be inhibited by an approved drug for NSCLC (e.g., a topoisomerase, for example, or a metalloprotease).

Tables 6 through 10 provides the protein name and corresponding UniProt identifier and any drugs that target the protein for five (5) different individuals (Subjects A, B, C, D and E). If no drugs are known to target the protein, then the table cell is left blank or contains the language “(None found)”. Further provided is the fold difference in expression of each protein in the individual as determined by the protein expression level in tumor tissue versus protein expression level in normal or healthy tissue from the same individual.

Table 6 shows a protein expression profile generated using compositions and methods of the invention from a single patient (Subject A) with lung cancer (adenocarcinoma). By way of example, the protein Lactotransferrin (UniProt P02788) was found to be down-regulated in tumor tissue about 10-fold (as expressed in the table as 0.1) relative to the same protein in normal or healthy tissue from the same individual. While at this time, this protein does not have a known drug, the Lactotransferin protein may be selected for drug development based on the differential expression levels between tumor tissue and healthy tissue.

By way of example, the protein Carbonic Anhydrase I (UnitProt 00915) was found to be down-regulated in tumor tissue about 7.7-fold (as expressed in the table as 0.13) relative to the same protein in normal or healthy tissue from the same individual. The Carbonic Anhydrase I has several known drug that target this protein (e.g., Hydrochlorothiazide, Quinethazone, Benzthiazide, Diazoxide, Trichlormethiazide, Methocarbamol, Amlodipine, Bendroflumethiazide, Brinzolamide, Dichlorphenamide, Methazolamide, Ethinamate, Hydroflumethiazide, Acetazolamide, Cyclothiazide, Zonisamide, Ethoxzolamide, Chlorothiazide, Methyclothiazide and Dorzolamide. Consequently, this individual may be responsive to a drug treatment plan that may include one or more of the drugs identified in the table 6. Thus, by way of example, a drug treatment plan for this individual may be developed by selecting one or more protein(s) that have differential expression between tumor tissue and healthy tissue of at least 7-fold (or at least 0.14 difference), and providing a drug treatment plan based on the drugs that target this particular protein.

By way of another example, the protein Hepatocyte Growth Factor or HGF (UniProt P08581) was found to be up-regulated in tumor tissues relative to normal or healthy tissue by about 7-fold (or 6.96 fold). This protein may be targeted by the drug Cabozantinib. Consequently, this individual may be responsive to a drug treatment plan that may include Cabozantinib. Thus, by way of example, a drug treatment plan for this individual may be developed by selecting one or more protein(s) that have differential expression between tumor tissue and healthy tissue of at least about 6 or 7-fold and providing a drug treatment plan based on the drugs that target this particular protein.

TABLE 6
Proteomic profile for a single individual (Subject A) based on proteins having at least a 4-fold difference in
expression between tumor tissue and normal tissue. Based on this threshold cut-off, this individual had 57 proteins
with at least a 4-fold (either up or down) difference in tumor to healthy tissue protein expression levels.

| | | | Protein |
|---|---|---|---|
| | | | Expression |
| UniProt | Protein Name | Drug List | (Tumor/Normal) |
| P02788 | Lactotransferrin | | 0.10 |
| P01008 | Antithrombin-III | Tinzaparin, Dalteparin, Nadroparin, Fondaparinux | 0.11 |
| | | sodium, Sulodexide, Ardeparin, Enoxaparin, Heparin | |
| P05186 | Alkaline | (None found) | 0.13 |
| | phosphatase, | | |
| | tissue-nonspecific | | |
| | isozyme | | |
| P00915 | Carbonic | Hydrochlorothiazide, Quinethazone, Benzthiazide, | 0.13 |
| | anhydrase I | Diazoxide, Trichlormethiazide, Methocarbamol, Amlodipine, | |
| | | Bendroflumethiazide, Brinzolamide, Dichlorphenamide, | |
| | | Methazolamide, Ethinamate, Hydroflumethiazide, | |
| | | Acetazolamide, Cyclothiazide, Zonisamide, Ethoxzolamide, | |
| | | Chlorothiazide, Methyclothiazide, Dorzolamide | |
| O43866 | CD5 antigen-like | | 0.13 |
| P05164 | Myeloperoxidase | Mesalazine, Melatonin, L-Carnitine, Cefdinir | 0.14 |
| P02775 | Connective-tissue | | 0.14 |
| | activating peptide | | |
| | III | | |
| P07451 | Carbonic | Zonisamide, Acetazolamide | 0.14 |
| | anhydrase 3 | | |
| P24158 | Myeloblastin | | 0.14 |
| P55774 | C-C motif | | 0.14 |
| | chemokine 18 | | |
| Q92563 | Testican-2 | | 0.14 |
| P02775 | Neutrophil- | | 0.15 |
| | activating peptide 2 | | |
| P22749 | Granulysin | | 0.15 |
| Q8NAC3 | Interleukin-17 | | 0.15 |
| | receptor C | | |
| P01024 | C3a anaphylatoxin | Intravenous Immunoglobulin | 0.16 |
| | des Arginine | | |
| O75594 | Peptidoglycan | | 0.17 |
| | recognition protein | | |
| P01024 | Complement C3 | Intravenous Immunoglobulin | 0.17 |
| P04040 | Catalase | Fomepizole | 0.17 |
| O75144 | B7 homolog 2 | | 0.17 |
| P01024 | Complement C3b, | Intravenous Immunoglobulin | 0.17 |
| | incactivated | | |
| Q14624 | Inter-alpha-trypsin | | 0.18 |
| | inhibitor heavy | | |
| | chain H4 | | |
| P03952 | Plasma kallikrein | | 0.18 |
| | (precursor) | | |
| P14780 | Matrix | Captopril, Glucosamine, Minocycline, Marimastat | 0.18 |
| | metalloproteinase- | | |
| | 9 | | |
| P0C0L4 | C4b-A | | 0.19 |
| P0C0L5 | | | |
| P00747 | Plasminogen | Streptokinase, Anistreplase, Aminocaproic Acid, | 0.20 |
| | | Urokinase, Reteplase, Alteplase, Aprotinin, | |
| | | Tranexamic Acid, Tenecteplase | |
| P01031 | Complement C5 | Eculizumab, Intravenous Immunoglobulin | 0.20 |
| P00740 | Coagulation factor | Menadione, Antihemophilic Factor | 0.20 |
| | IX (activated | | |
| | form) | | |
| P02671 | Fibrinogen alpha, | | 0.20 |
| P02675 | beta, and gamma | | |
| P02679 | chains | | |
| P02776 | Platelet factor 4 | Drotrecogin alfa | 0.20 |
| P07225 | Vitamin K- | Menadione, Sodium Tetradecyl Sulfate, Drotrecogin alfa | 0.21 |
| | dependent protein | | |
| | S | | |
| Q15109 | Advanced | | 0.22 |
| | glycosylation end | | |
| | product-specific | | |
| | receptor | | |
| P27918 | Properdin | | 0.23 |
| P07288, | Prostate-specific | | 0.23 |
| P01011 | antigen and Alpha- | | |
| | 1-antichymotrypsin | | |
| P48061 | Stromal cell- | Tinzaparin | 0.23 |
| | derived factor 1 | | |
| P00747 | Angiostatin | Streptokinase, Anistreplase, Aminocaproic Acid, | 0.23 |
| | | Urokinase, Reteplase, Alteplase, Aprotinin, | |
| | | Tranexamic Acid, Tenecteplase | |
| Q16627 | C-C motif | | 0.23 |
| | chemokine 14 | | |
| P02647 | Apolipoprotein A-I | | 0.23 |
| Q04756 | Hepatocyte growth | | 0.23 |
| | factor activator | | |
| Q15848 | Adiponectin | | 0.24 |
| Q9Y5Y7 | Lymphatic vessel | | 0.24 |
| | endothelial | | |
| | hyaluronic acid | | |
| | receptor 1 | | |
| P00740 | Coagulation factor | Menadione, Antihemophilic Factor | 0.24 |
| | IX | | |
| P10646 | Tissue factor | Dalteparin, Coagulation factor VIIa | 0.25 |
| | pathway inhibitor | | |
| P01042 | Kininogen-1, | | 0.25 |
| | HMW | | |
| P26927 | Hepatocyte growth | | 0.25 |
| | factor-like protein | | |
| | precursor | | |
| P02735 | Serum amyloid A | | 4.11 |
| | protein | | |
| P24821 | Tenascin | | 4.13 |
| Q13554 | Calcium/calmodulin- | | 4.32 |
| | dependent protein | | |
| | kinase type | | |
| | II beta chain | | |
| Q13557 | Calcium/calmodulin- | | 4.51 |
| | dependent protein | | |
| | kinase type | | |
| | II delta chain | | |
| P21741 | Midkine | | 5.15 |
| Q9UQM7 | Calcium/calmodulin- | | 5.27 |
| | dependent protein | | |
| | kinase type | | |
| | II alpha chain | | |
| Q6UXM1 | Leucine-rich | | 5.66 |
| | repeats and | | |
| | immunoglobulin- | | |
| | like domains | | |
| | protein 3 | | |
| P08581 | Hepatocyte growth | Cabozantinib | 6.96 |
| | factor receptor | | |
| Q8N1Q1 | Carbonic | Zonisamide | 7.42 |
| | anhydrase 13 | | |
| Q9UJ71 | C-type lectin | | 7.73 |
| | domain family 4 | | |
| | member K | | |
| P18669 | Phosphoglycerate | | 12.24 |
| | mutase 1 | | |
| Q07654 | Trefoil factor 3 | | 14.60 |
| P35442 | Thrombospondin-2 | | 16.96 |

In summary, the general approach described above may be applied to anyone of the protein-drug combinations described in Table 6 to develop a drug treatment plan or to administer the drug or drugs to the individual based on their proteomic profile (differential protein expression levels—“up” or “down” and the fold-level of that difference). Further, the approach may be used to identify proteins that may be drug targets for the treatment of individuals or groups of individuals that may share the same protein differential expression profile or profile range (i.e., have at least about a 4-fold, 5-fold, 6-fold, 7-fold, 8-fold and up to 100-fold or more in expression difference of the same protein as between tumor tissue and healthy/normal tissue).

Table 7 shows a protein expression profile generated using compositions and methods of the invention from a single patient (Subject B) with lung cancer (adenocarcinoma). By way of example, the protein Tryptase-beta-2 (UniProt P20231) was found to be down-regulated in tumor tissue about 33-fold (as expressed in the table as 0.03) relative to the same protein in normal or healthy tissue from the same individual. While at this time, this protein does not have a known drug, the Tryptase-beta-2 protein may be selected for drug development based on the differential expression levels between tumor tissue and healthy tissue.

By way of example, the protein Carbonic Anhydrase 3 (UniProt P07451) was found to be down-regulated in tumor tissue about 25-fold (as expressed in the table as 0.04) relative to the same protein in normal or healthy tissue from the same individual. The Carbonic Anhydrase 3 has known drugs that target this protein (e.g., Zonisamide and Acetazolamide). Consequently, this individual may be responsive to a drug treatment plan that may include Zonisamide and/or Acetazolamide. Thus, by way of example, a drug treatment plan for this individual may be developed by selecting one or more protein(s) that have differential expression between tumor tissue and healthy tissue of at least 25-fold (or at least 0.04 difference), and providing a drug treatment plan based on the drugs that target this particular protein.

By way of another example, the protein C3a anaphylatoxin (UniProt P01024) was found to be up-regulated in tumor tissues relative to normal or healthy tissue by about 49-fold (or 49.04 fold). This protein may be targeted by the drug Intravenous Immunoglobulin. Consequently, this individual may be responsive to a drug treatment plan that may include Intravenous Immunoglobulin. Thus, by way of example, a drug treatment plan for this individual may be developed by selecting one or more protein(s) that have differential expression between tumor tissue and healthy tissue of at least about 49-fold and providing a drug treatment plan based on the drugs that target this particular protein.

TABLE 7
Proteomic profile for a single individual (Subject B) based on proteins having at least
a 4-fold difference in expression between tumor tissue and normal tissue. Based on this
threshold cut-off, this individual had 69 proteins with at least a 4-fold (either up
or down) difference in tumor to healthy tissue protein expression levels.

| | | | Protein |
|---|---|---|---|
| | | | Expression |
| Uniprot | Protein Name: | Drug List: | (Tumor/Normal) |
| Q15109 | Advanced glycosylation end | (None found) | 0.01 |
| | product-specific receptor | | |
| P20231 | Tryptase beta-2 | (None found) | 0.03 |
| P07451 | Carbonic anhydrase 3 | Zonisamide, Acetazolamide | 0.04 |
| Q9HCB6 | Spondin-1 | (None found) | 0.05 |
| P01833 | Polymeric | (None found) | 0.08 |
| | immunoglobulin | | |
| | receptor | | |
| P62826 | GTP-binding nuclear | (None found) | 0.10 |
| | protein Ran | | |
| P13686 | Tartrate-resistant acid | (None found) | 0.11 |
| | phosphatase type 5 | | |
| P11387 | DNA topoisomerase | Irinotecan, Topotecan, Lucanthone, | 0.13 |
| | 1 | Sodium stibogluconate | |
| Q06187 | Tyrosine-protein | (None found) | 0.14 |
| | kinase BTK | | |
| P07483 | Fatty acid-binding | (None found) | 0.14 |
| | protein, heart_RAT | | |
| Q96KQ7 | Histone-lysine N- | (None found) | 0.15 |
| | methyltransferase, | | |
| | H3 lysine-9 specific 3 | | |
| #N/A | Creatine kinase B- | (None found) | 0.15 |
| | type, Creatine kinase | | |
| | M-type | | |
| P02776 | Platelet factor 4 | Drotrecogin alfa | 0.17 |
| Q8NAC3 | Interleukin-17 | (None found) | 0.17 |
| | receptor C | | |
| Q13557 | Calcium/calmodulin- | (None found) | 0.20 |
| | dependent protein | | |
| | kinase type II delta | | |
| | chain | | |
| P02775 | Neutrophil-activating | (None found) | 0.20 |
| | peptide 2 | | |
| P07585 | Decorin | (None found) | 0.20 |
| P48061 | Stromal cell-derived | Tinzaparin | 0.20 |
| | factor 1 | | |
| P02775 | Connective-tissue | (None found) | 0.20 |
| | activating peptide III | | |
| P20718 | Granzyme H | (None found) | 0.21 |
| #N/A | Glycogen synthase | (None found) | 0.21 |
| | kinase-3 alpha/beta | | |
| P00568 | Myokinase, human | (None found) | 0.22 |
| P09211 | Glutathione S- | Glutathione, Clomipramine | 0.22 |
| | transferase Pi | | |
| Q9UQM7 | Calcium/calmodulin- | (None found) | 0.23 |
| | dependent protein | | |
| | kinase type II alpha | | |
| | chain | | |
| Q13219 | Pappalysin-1 | (None found) | 0.23 |
| Q02083 | N-acylethanolamine- | (None found) | 0.23 |
| | hydrolyzing acid | | |
| | amidase | | |
| Q13554 | Calcium/calmodulin- | (None found) | 0.23 |
| | dependent protein | | |
| | kinase type II beta | | |
| | chain | | |
| P62081 | 40S ribosomal | (None found) | 0.23 |
| | protein S7 | | |
| P21860 | Receptor tyrosine- | (None found) | 0.24 |
| | protein kinase erbB-3 | | |
| Q9UIK4 | Death-associated | (None found) | 0.24 |
| | protein kinase 2 | | |
| O95219 | Sorting nexin-4 | (None found) | 0.24 |
| Q99983 | Osteomodulin | (None found) | 4.02 |
| O75144 | B7 homolog 2 | (None found) | 4.02 |
| Q76M96 | Coiled-coil domain- | (None found) | 4.35 |
| | containing protein 80 | | |
| Q9UBT3 | Dickkopf-related | (None found) | 4.52 |
| | protein 4 | | |
| P02741 | C-reactive protein | inhaled insulin | 4.59 |
| P24821 | Tenascin | (None found) | 4.64 |
| Q9GZN4 | Brain-specific serine | (None found) | 4.77 |
| | protease 4 | | |
| P02788 | Lactotransferrin | (None found) | 5.04 |
| O15123 | Angiopoietin-2 | (None found) | 5.22 |
| P80188 | Neutrophil | (None found) | 5.26 |
| | gelatinase-associated | | |
| | lipocalin | | |
| P18428 | Lipopolysaccharide- | (None found) | 5.39 |
| | binding protein | | |
| P09237 | Matrilysin | Marimastat | 5.81 |
| P0C0S5 | Histone H2A.z | (None found) | 6.56 |
| P14780 | Matrix | Captopril, Glucosamine, | 6.68 |
| | metalloproteinase-9 | Minocycline, Marimastat | |
| O94907 | Dickkopf-related | (None found) | 7.19 |
| | protein 1 | | |
| P08476 | Inhibin beta A chain | (None found) | 7.90 |
| P20160 | Azurocidin | (None found) | 8.29 |
| O75509 | Death receptor 6 | (None found) | 8.80 |
| P98066 | TNF-stimulated gene | (None found) | 8.80 |
| | 6 protein | | |
| P42702 | Leukemia inhibitory | (None found) | 9.51 |
| | factor soluble | | |
| | receptor (secreted) | | |
| P02751 | Fibronectin-1 | Ocriplasmin | 10.22 |
| | Fragment 4 | | |
| Q9HD89 | Resistin | (None found) | 10.54 |
| P02768 | Serum albumin | (None found) | 10.78 |
| P01033 | Metalloproteinase | (None found) | 12.20 |
| | inhibitor 1 | | |
| P02751 | Fibronectin | Ocriplasmin | 12.95 |
| P78380 | Oxidized low-density | (None found) | 14.13 |
| | lipoprotein receptor 1 | | |
| P05164 | Myeloperoxidase | Mesalazine, Melatonin, | 14.42 |
| | | L-Carnitine, Cefdinir | |
| P17213 | Bactericidal | (None found) | 17.67 |
| | permeability- | | |
| | increasing protein | | |
| P02751 | Fibronectin-1 | Ocriplasmin | 18.73 |
| | Fragment 3 | | |
| P05121 | Plasminogen | Anistreplase, Urokinase, | 20.02 |
| | | Reteplase, Alteplase, | |
| | activator inhibitor 1 | Tenecteplase, Drotrecogin alfa | |
| P52823 | Stanniocalcin-1 | (None found) | 20.90 |
| O75594 | Peptidoglycan | (None found) | 22.73 |
| | recognition protein | | |
| P03956 | MMP-1 | Marimastat | 23.09 |
| P02778 | Small-inducible | (None found) | 27.72 |
| | cytokine B10 | | |
| P35442 | Thrombospondin-2 | (None found) | 39.69 |
| P10145 | Interleukin-8 | (None found) | 42.05 |
| P01024 | C3a anaphylatoxin | Intravenous Immunoglobulin | 49.04 |
| P39900 | Macrophage | Acetohydroxamic Acid, Marimastat | 116.16 |
| | metalloelastase | | |

In summary, the general approach described above may be applied to anyone of the protein-drug combinations described in Table 7 to develop a drug treatment plan or to administer the drug or drugs to the individual based on their proteomic profile (differential protein expression levels—“up” or “down” and the fold-level of that difference). Further, the approach may be used to identify proteins that may be drug targets for the treatment of individuals or groups of individuals that may share the same protein differential expression profile or profile range (i.e., have at least about a 4-fold, 5-fold, 6-fold, 7-fold, 8-fold and up to 100-fold or more in expression difference of the same protein as between tumor tissue and healthy/normal tissue).

Table 8 shows a protein expression profile generated using compositions and methods of the invention from a single patient (Subject C) with lung cancer (adenocarcinoma). By way of example, the protein Advanced glycosylation end product-specific receptor (UniProt Q15109) was found to be down-regulated in tumor tissue about 100-fold (as expressed in the table as 0.01) relative to the same protein in normal or healthy tissue from the same individual. While at this time, this protein does not have a known drug, the Advanced glycosylation end product-specific receptor protein may be selected for drug development based on the differential expression levels between tumor tissue and healthy tissue.

By way of example, the protein Coagulation Factor X (UniProt P00742) was found to be down-regulated in tumor tissue about 5-fold (as expressed in the table as 0.2) relative to the same protein in normal or healthy tissue from the same individual. The Coagulation Factor X has known drugs that target this protein (e.g., Fondaparinux sodium, Menadione, Enoxaparin, Coagulation factor VIIa, Antihemophilic Factor, Rivaroxaban, Apixaban, Coagulation Factor IX and Heparin). Consequently, this individual may be responsive to a drug treatment plan that may include Zonisamide and/or Acetazolamide. Thus, by way of example, a drug treatment plan for this individual may be developed by selecting one or more protein(s) that have differential expression between tumor tissue and healthy tissue of at least 5-fold (or at least 0.2 difference), and providing a drug treatment plan based on the drugs that target this particular protein.

By way of another example, the protein Matrilysin (UniProt P09237) was found to be up-regulated in tumor tissues relative to normal or healthy tissue by about 5-fold (or 5.23 fold). This protein may be targeted by the drug Marimastat. Consequently, this individual may be responsive to a drug treatment plan that may include Marimastat. Thus, by way of example, a drug treatment plan for this individual may be developed by selecting one or more protein(s) that have differential expression between tumor tissue and healthy tissue of at least about 5-fold and providing a drug treatment plan based on the drugs that target this particular protein.

TABLE 8
Proteomic profile for a single individual (Subject C) based on proteins having at least a 4-fold difference in
expression between tumor tissue and normal tissue. Based on this threshold cut-off, this individual had 86 proteins
with at least a 4-fold (either up or down) difference in tumor to healthy tissue protein expression levels.

| | | | Protein |
|---|---|---|---|
| | | | Expression |
| Uniprot | Protein Name: | Drug List: | (Tumor/Normal) |
| Q15109 | Advanced | (None found) | 0.01 |
| | glycosylation | | |
| | end product- | | |
| | specific receptor | | |
| P02787 | Serotransferrin | Aluminium | 0.05 |
| P21810 | Biglycan | (None found) | 0.09 |
| O43866 | CD5 antigen-like | (None found) | 0.09 |
| Q14624 | Inter-alpha- | (None found) | 0.10 |
| | trypsin inhibitor | | |
| | heavy chain H4 | | |
| Q8NAC3 | Interleukin-17 | (None found) | 0.11 |
| | receptor C | | |
| P00915 | Carbonic | Hydrochlorothiazide, Quinethazone, Benzthiazide, | 0.12 |
| | anhydrase I | Diazoxide, Trichlormethiazide, Methocarbamol, Amlodipine, | |
| | | Bendroflumethiazide, Brinzolamide, Dichlorphenamide, | |
| | | Methazolamide, Ethinamate, Hydroflumethiazide, Acetazolamide, | |
| | | Cyclothiazide, Zonisamide, Ethoxzolamide, Chlorothiazide, | |
| | | Methyclothiazide, Dorzolamide | |
| P02647 | Apolipoprotein | (None found) | 0.12 |
| | A-I | | |
| P02775 | Neutrophil- | (None found) | 0.13 |
| | activating | | |
| | peptide 2 | | |
| P04040 | Catalase | Fomepizole | 0.13 |
| Q04756 | Hepatocyte | (None found) | 0.13 |
| | growth factor | | |
| | activator | | |
| P01042 | Kininogen-1, | (None found) | 0.14 |
| | HMW | | |
| P03952 | Plasma kallikrein | (None found) | 0.14 |
| | (precursor) | | |
| P05164 | Myeloperoxidase | Mesalazine, Melatonin, L-Carnitine, Cefdinir | 0.14 |
| P02775 | Connective- | (None found) | 0.14 |
| | tissue activating | | |
| | peptide III | | |
| O75594 | Peptidoglycan | (None found) | 0.14 |
| | recognition | | |
| | protein | | |
| P07451 | Carbonic | Zonisamide, Acetazolamide | 0.14 |
| | anhydrase 3 | | |
| P02788 | Lactotransferrin | (None found) | 0.14 |
| #N/A | C4b-A | (None found) | 0.15 |
| P05546 | Heparin cofactor 2 | Ardeparin, Sulodexide | 0.15 |
| P01031 | Complement C5 | Eculizumab, Intravenous Immunoglobulin | 0.15 |
| P01019 | Angiotensinogen | (None found) | 0.15 |
| P07585 | Decorin | (None found) | 0.15 |
| P00747 | Plasminogen | Streptokinase, Anistreplase, Aminocaproic Acid, | 0.15 |
| | | Urokinase, Reteplase, Alteplase, Aprotinin, | |
| | | Tranexamic Acid, Tenecteplase | |
| P43652 | Afamin | (None found) | 0.16 |
| P04278 | Sex hormone- | (None found) | 0.16 |
| | binding globulin | | |
| P07225 | Vitamin K- | Menadione, Sodium Tetradecyl Sulfate, Drotrecogin alfa | 0.16 |
| | dependent | | |
| | protein S | | |
| #N/A | Fibrinogen | (None found) | 0.16 |
| | alpha, beta, and | | |
| | gamma chains | | |
| P02790 | Hemopexin | (None found) | 0.16 |
| Q9Y5Y7 | Lymphatic | (None found) | 0.16 |
| | vessel | | |
| | endothelial | | |
| | hyaluronic acid | | |
| | receptor 1 | | |
| P05543 | Thyroxine- | (None found) | 0.16 |
| | binding globulin | | |
| Q9UGM5 | Fetuin-B | (None found) | 0.16 |
| P27918 | Properdin | (None found) | 0.16 |
| Q15848 | Adiponectin | (None found) | 0.17 |
| P12259 | Coagulation | ART-123, Drotrecogin alfa | 0.17 |
| | Factor V | | |
| P00751 | Complement | (None found) | 0.17 |
| | factor B | | |
| P05186 | Alkaline | (None found) | 0.17 |
| | phosphatase, | | |
| | tissue- | | |
| | nonspecific | | |
| | isozyme | | |
| P00747 | Angiostatin | Streptokinase, Anistreplase, Aminocaproic Acid, | 0.17 |
| | | Urokinase, Reteplase, Alteplase, Aprotinin, | |
| | | Tranexamic Acid, Tenecteplase | |
| P04196 | Histidine-rich | (None found) | 0.18 |
| | glycoprotein | | |
| P16671 | Platelet | (None found) | 0.18 |
| | glycoprotein 4 | | |
| P02649 | Apolipoprotein E | Serum albumin iodonated, Human Serum Albumin | 0.18 |
| | (isoform E3) | | |
| P06681 | Complement C2 | (None found) | 0.18 |
| P01024 | Complement | Intravenous Immunoglobulin | 0.18 |
| | C3b, incactivated | | |
| Q01638 | Interleukin-1 | (None found) | 0.19 |
| | receptor-like 1 | | |
| P07483 | Fatty acid- | (None found) | 0.19 |
| | binding protein, | | |
| | heart RAT | | |
| P00742 | Coagulation | Fondaparinux sodium, Menadione, Enoxaparin, Coagulation | 0.19 |
| | factor X | factor VIIa, Antihemophilic Factor, Rivaroxaban, | |
| | (activated form) | Apixaban, Coagulation Factor IX, Heparin | |
| P02748 | Complement | (None found) | 0.19 |
| | component C9 | | |
| P01008 | Antithrombin-III | Tinzaparin, Dalteparin, Nadroparin, Fondaparinux | 0.19 |
| | | sodium, Sulodexide, Ardeparin, Enoxaparin, Heparin | |
| #N/A | Complement | (None found) | 0.19 |
| | C1q | | |
| | subcomponent | | |
| | subunits A, B, | | |
| | and C | | |
| P26927 | Hepatocyte | (None found) | 0.19 |
| | growth factor- | | |
| | like protein | | |
| | precursor | | |
| P08603 | Complement | (None found) | 0.19 |
| | factor H | | |
| P02649 | Apolipoprotein E | Serum albumin iodonated, Human Serum Albumin | 0.19 |
| | (isoform E2) | | |
| #N/A | Creatine kinase | (None found) | 0.19 |
| | B-type, Creatine | | |
| | kinase M-type | | |
| P01042 | Kininogen-1, | (None found) | 0.19 |
| | HMW | | |
| Q96IY4 | Carboxypeptidase | (None found) | 0.20 |
| | B2 | | |
| P29622 | Kallistatin | (None found) | 0.20 |
| P00742 | Coagulation | Fondaparinux sodium, Menadione, Enoxaparin, Coagulation | 0.20 |
| | Factor X | factor VIIa, Antihemophilic Factor, Rivaroxaban, Apixaban, | |
| | | Coagulation Factor IX, Heparin | |
| P02748 | Complement | (None found) | 0.21 |
| | component C9 | | |
| P17213 | Bactericidal | (None found) | 0.21 |
| | permeability- | | |
| | increasing | | |
| | protein | | |
| P01011 | Alpha-1- | (None found) | 0.21 |
| | antichymotrypsin | | |
| P13671 | Complement | (None found) | 0.22 |
| | component C6 | | |
| P24592 | Insulin-like | (None found) | 0.22 |
| | growth factor- | | |
| | binding protein 6 | | |
| #N/A | Complement | (None found) | 0.22 |
| | C5b, and | | |
| | Complement | | |
| | component C6 | | |
| P01023 | Alpha-2- | Ocriplasmin, Bacitracin, Becaplermin | 0.22 |
| | macroglobulin | | |
| P05156 | Complement | (None found) | 0.22 |
| | factor I | | |
| P24158 | Myeloblastin | (None found) | 0.23 |
| P35247 | Pulmonary | (None found) | 0.23 |
| | surfactant- | | |
| | associated | | |
| | protein D | | |
| P03951 | Coagulation | Coagulation Factor IX | 0.23 |
| | factor XI | | |
| P22749 | Granulysin | (None found) | 0.23 |
| P01024 | Complement C3 | Intravenous Immunoglobulin | 0.23 |
| P05154 | Plasma serine | Urokinase, Drotrecogin alfa | 0.24 |
| | protease | | |
| | inhibitor | | |
| P01024 | C3a | Intravenous Immunoglobulin | 0.24 |
| | anaphylatoxin | | |
| | des Arginine | | |
| #N/A | Ferritin heavy | (None found) | 0.24 |
| | and light chains | | |
| Q8N474 | Secreted | (None found) | 0.24 |
| | frizzled-related | | |
| | protein 1 | | |
| P14543 | Nidogen-1 | Urokinase | 0.24 |
| P18428 | Lipopolysac- | (None found) | 0.24 |
| | charide-binding | | |
| | protein | | |
| P02649 | Apolipoprotein E | Serum albumin iodonated, Human Serum Albumin | 0.24 |
| | (isoform E4) | | |
| P02775 | Neutrophil- | (None found) | 0.25 |
| | activating | | |
| | peptide 2 | | |
| P11387 | DNA | Irinotecan, Topotecan, Lucanthone, Sodium | 4.03 |
| | topoisomerase 1 | stibogluconate | |
| Q99731 | Small-inducible | (None found) | 4.13 |
| | cytokine A19 | | |
| P53582 | Methionine | Nitroxoline | 4.19 |
| | aminopeptidase 1 | | |
| P09237 | Matrilysin | Marimastat | 5.23 |
| P01833 | Polymeric | (None found) | 6.23 |
| | immunoglobulin | | |
| | receptor | | |
| Q9H773 | XTP3- | (None found) | 8.25 |
| | transactivated | | |
| | gene A protein | | |
| P35442 | Thrombospondin- | (None found) | 9.83 |
| | 2 | | |
| O43927 | Small-inducible | (None found) | 26.02 |
| | cytokine B13 | | |

In summary, the general approach described above may be applied to anyone of the protein-drug combinations described in Table 8 to develop a drug treatment plan or to administer the drug or drugs to the individual based on their proteomic profile (differential protein expression levels—“up” or “down” and the fold-level of that difference). Further, the approach may be used to identify proteins that may be drug targets for the treatment of individuals or groups of individuals that may share the same protein differential expression profile or profile range (i.e., have at least about a 4-fold, 5-fold, 6-fold, 7-fold, 8-fold and up to 100-fold or more in expression difference of the same protein as between tumor tissue and healthy/normal tissue).

Table 9 shows a protein expression profile generated using compositions and methods of the invention from a single patient (Subject D) with lung cancer (squamous carcinoma). By way of example, the protein Mitogen-activated protein kinase 13 (UniProt O15264) was found to be up-regulated in tumor tissue about 4-fold (or 4.03-fold) relative to the same protein in normal or healthy tissue from the same individual. While at this time, this protein does not have a known drug, the Mitogen-activated protein kinase 13 protein may be selected for drug development based on the differential expression levels between tumor tissue and healthy tissue.

By way of example, the protein Heparin-binding growth factor 2 (UniProt P09038 was found to be down-regulated in tumor tissue about 4-fold (as expressed in the table as 0.24) relative to the same protein in normal or healthy tissue from the same individual. The Heparin-binding growth factor 2 has known drugs that target this protein (e.g., Pentosan Polysulfate, Sucralfate and Sirolimus). Consequently, this individual may be responsive to a drug treatment plan that may include Pentosan Polysulfate, Sucralfate and/or Sirolimus. Thus, by way of example, a drug treatment plan for this individual may be developed by selecting one or more protein(s) that have differential expression between tumor tissue and healthy tissue of at least 4-fold (or at least 0.24 difference), and providing a drug treatment plan based on the drugs that target this particular protein.

By way of another example, the protein Plasminogen activator inhibitor 1 (UniProt P05121) was found to be up-regulated in tumor tissues relative to normal or healthy tissue by about 182-fold (or 181.88 fold). The Plasminogen activator inhibitor 1 has known drugs that target this protein (e.g., Anistreplase, Urokinase, Reteplase, Alteplase, Tenecteplase and Drotrecogin alfa). Consequently, this individual may be responsive to a drug treatment plan that may include Anistreplase, Urokinase, Reteplase, Alteplase, Tenecteplase and/or Drotrecogin alfa. Thus, by way of example, a drug treatment plan for this individual may be developed by selecting one or more protein(s) that have differential expression between tumor tissue and healthy tissue of at least about 182-fold and providing a drug treatment plan based on the drugs that target this particular protein.

TABLE 9
Proteomic profile for a single individual (Subject D) based on proteins having at least
a 4-fold difference in expression between tumor tissue and normal tissue. Based on this
threshold cut-off, this individual had 95 proteins with at least a 4-fold (either up
or down) difference in tumor to healthy tissue protein expression levels.

| | | | Protein |
|---|---|---|---|
| | | | Expression |
| Uniprot | Protein Name: | Drug List: | (Tumor/Normal) |
| Q15109 | Advanced | (None found) | 0.02 |
| | glycosylation end | | |
| | product-specific | | |
| | receptor | | |
| P20231 | Tryptase beta-2 | (None found) | 0.03 |
| #N/A | Complement C1q | (None found) | 0.04 |
| | subcomponent | | |
| | subunits A, B, and C | | |
| P01833 | Polymeric | (None found) | 0.04 |
| | immunoglobulin | | |
| | receptor | | |
| #N/A | Creatine kinase B- | (None found) | 0.05 |
| | type, Creatine kinase | | |
| | M-type | | |
| P07451 | Carbonic anhydrase 3 | Zonisamide, Acetazolamide | 0.06 |
| P06732 | Creatine kinase | Creatine | 0.09 |
| | M-type | | |
| P21810 | Biglycan | (None found) | 0.10 |
| P12259 | Coagulation Factor V | ART-123, Drotrecogin alfa | 0.11 |
| #N/A | Complement C4-A | (None found) | 0.11 |
| | and Complement | | |
| | C4-B | | |
| #N/A | Complement C5b, | (None found) | 0.12 |
| | and Complement | | |
| | component C6 | | |
| P01037 | Cystatin-SN | (None found) | 0.12 |
| P00742 | Coagulation factor X | Fondaparinux sodium, Menadione, Enoxaparin, | 0.14 |
| | (activated form) | Coagulation factor VIIa, Antihemophilic | |
| | | Factor, Rivaroxaban, Apixaban, Coagulation | |
| | | Factor IX, Heparin | |
| P02679 | Fibrinogen g-chain | Sucralfate | 0.14 |
| | dimer | | |
| P00742 | Coagulation Factor X | Fondaparinux sodium, Menadione, Enoxaparin, | 0.15 |
| | | Coagulation factor VIIa, Antihemophilic | |
| | | Factor, Rivaroxaban, Apixaban, Coagulation | |
| | | Factor IX, Heparin | |
| Q9HCB6 | Spondin-1 | (None found) | 0.16 |
| P00736 | Complement C1r | Alemtuzumab, Daclizumab, Ibritumomab, | 0.16 |
| | subcomponent | Trastuzumab, Bevacizumab, Efalizumab, | |
| | | Muromonab, Adalimumab, Palivizumab, | |
| | | Abciximab, Natalizumab, Basiliximab, | |
| | | Cetuximab, Rituximab, Gemtuzumab ozogamicin, | |
| | | Etanercept, Tositumomab, Alefacept | |
| P07585 | Decorin | (None found) | 0.16 |
| P04275 | von Willebrand | Antihemophilic Factor | 0.16 |
| | factor | | |
| P78423 | Fractalkine | (None found) | 0.16 |
| #N/A | Immunoglobulin G | (None found) | 0.16 |
| P00740 | Coagulation factor IX | Menadione, Antihemophilic Factor | 0.16 |
| | (activated form) | | |
| P35625 | Metalloproteinase | (None found) | 0.16 |
| | inhibitor 3 | | |
| O75914 | Serine/threonine- | (None found) | 0.17 |
| | protein kinase PAK 3 | | |
| P00740 | Coagulation factor IX | Menadione, Antihemophilic Factor | 0.17 |
| P02735 | Serum amyloid A | (None found) | 0.18 |
| | protein | | |
| P01031 | Complement C5 | Eculizumab, Intravenous Immunoglobulin | 0.18 |
| P00746 | Complement factor D | (None found) | 0.18 |
| Family | None | (None found) | 0.18 |
| P07483 | Fatty acid-binding | (None found) | 0.18 |
| | protein, heart RAT | | |
| Q9UMF0 | Intercellular adhesion | (None found) | 0.19 |
| | molecule 5 | | |
| P24158 | Myeloblastin | (None found) | 0.19 |
| O43866 | CD5 antigen-like | (None found) | 0.19 |
| #N/A | D-dimer | (None found) | 0.19 |
| P16671 | Platelet | (None found) | 0.19 |
| | glycoprotein 4 | | |
| P13686 | Tartrate-resistant acid | (None found) | 0.19 |
| | phosphatase type 5 | | |
| P03952 | Plasma kallikrein | (None found) | 0.19 |
| | (precursor) | | |
| P02775 | Neutrophil-activating | (None found) | 0.19 |
| | peptide 2 | | |
| Q8NAC3 | Interleukin-17 | (None found) | 0.20 |
| | receptor C | | |
| #N/A | Fibrinogen alpha, | (None found) | 0.20 |
| | beta, and gamma | | |
| | chains | | |
| P08246 | Leukocyte elastase | Pegfilgrastim, Filgrastim, Alpha-1- | 0.20 |
| | | proteinase inhibitor | |
| Q07507 | Dermatopontin | (None found) | 0.21 |
| P13671 | Complement | (None found) | 0.21 |
| | component C6 | | |
| P00751 | Complement factor B | (None found) | 0.21 |
| Q9BU40 | Chordin-like | (None found) | 0.21 |
| | protein 1 | | |
| Q14515 | SPARC-like | (None found) | 0.22 |
| | protein 1 | | |
| P07225 | Vitamin K-dependent | Menadione, Sodium Tetradecyl | 0.23 |
| | protein S | Sulfate, Drotrecogin alfa | |
| P23280 | Carbonic anhydrase 6 | Zonisamide | 0.23 |
| P07339 | Cathepsin D | Insulin, Insulin Regular | 0.23 |
| P02748 | Complement | (None found) | 0.23 |
| | component C9 | | |
| Q12860 | contactin-1 | (None found) | 0.24 |
| P09038 | Heparin-binding | Pentosan Polysulfate, Sucralfate, Sirolimus | 0.24 |
| | growth factor 2 | | |
| Q96IY4 | Carboxypeptidase B2 | (None found) | 0.25 |
| O15264 | Mitogen-activated | (None found) | 4.03 |
| | protein kinase 13 | | |
| P24593 | Insulin-like growth | (None found) | 4.13 |
| | factor-binding | | |
| | protein 5 | | |
| O14929 | Histone | (None found) | 4.19 |
| | acetyltransferase type | | |
| | B catalytic subunit | | |
| P05164 | Myeloperoxidase | Mesalazine, Melatonin, L-Carnitine, | 4.26 |
| | | Cefdinir | |
| P04818 | TS | Pemetrexed, Trimethoprim, Fluorouracil, | 4.28 |
| | | Leucovorin, Gemcitabine, Pralatrexate, Capecitabine, | |
| | | Raltitrexed, Trifluridine, Floxuridine | |
| P52292 | Importin subunit | (None found) | 4.32 |
| | alpha-2 | | |
| O43291 | Kunitz-type protease | (None found) | 4.39 |
| | inhibitor 2 | | |
| P17936 | Insulin-like growth | Mecasermin | 4.54 |
| | factor-binding | | |
| | protein 3 | | |
| P02768 | Serum albumin | (None found) | 4.55 |
| Q76M96 | Coiled-coil domain- | (None found) | 4.88 |
| | containing protein 80 | | |
| O75509 | Death receptor 6 | (None found) | 4.99 |
| P00533 | Epidermal growth | Trastuzumab, Lidocaine, Lapatinib, Afatinib, | 5.10 |
| | factor receptor | Panitumumab, Gefitinib, Cetuximab, Erlotinib, | |
| | | Vandetanib | |
| O60911 | Cathepsin L2 | (None found) | 5.20 |
| P01033 | Metalloproteinase | (None found) | 5.59 |
| | inhibitor 1 | | |
| Q03154 | Aminoacylase-1 | L-Aspartic Acid | 5.63 |
| Q9NQU5 | Serine/threonine- | (None found) | 5.72 |
| | protein kinase PAK 6 | | |
| P10619 | Lysosomal protective | (None found) | 5.79 |
| | protein | | |
| P32004 | Neural cell adhesion | (None found) | 6.10 |
| | molecule L1 | | |
| P05067 | Amyloid beta A4 | (None found) | 6.16 |
| | protein | | |
| P31947 | 14-3-3 protein sigma | (None found) | 6.32 |
| P25787 | Proteasome subunit | (None found) | 6.35 |
| | alpha type 2 | | |
| P18669 | Phosphoglycerate | (None found) | 6.65 |
| | mutase 1 | | |
| P02778 | Small-inducible | (None found) | 8.01 |
| | cytokine B10 | | |
| P08174 | Complement decay- | Chloramphenicol | 8.11 |
| | accelerating factor | | |
| P19957 | Elafin | (None found) | 8.98 |
| P05231 | Interleukin-6 | Ginseng | 9.18 |
| P02751 | Fibronectin | Ocriplasmin | 9.54 |
| #N/A | Cell division control | (None found) | 9.71 |
| | protein 2 homolog, | | |
| | G2/mitotic-specific | | |
| | cyclin-B1 Complex | | |
| P35442 | Thrombospondin-2 | (None found) | 10.23 |
| Q29980 | MHC class I | (None found) | 13.84 |
| | polypeptide-related | | |
| | sequence B | | |
| O95633 | Follistatin-related | (None found) | 14.08 |
| | protein 3 | | |
| P12830 | Epithelial cadherin | (None found) | 14.21 |
| P02751 | Fibronectin-1 | Ocriplasmin | 14.37 |
| | Fragment 3 | | |
| Q9H773 | XTP3-transactivated | (None found) | 14.43 |
| | gene A protein | | |
| P02751 | Fibronectin-1 | Ocriplasmin | 15.27 |
| | Fragment 4 | | |
| Q03167 | TGF-beta receptor | (None found) | 15.89 |
| | type III | | |
| P99999 | Cytochrome c | Minocycline | 16.91 |
| P78380 | Oxidized low-density | (None found) | 19.32 |
| | lipoprotein receptor 1 | | |
| P52823 | Stanniocalcin-1 | (None found) | 34.39 |
| Q9GZN4 | Brain-specific serine | (None found) | 36.50 |
| | protease 4 | | |
| P39900 | Macrophage | Acetohydroxamic Acid, Marimastat | 37.71 |
| | metalloelastase | | |
| P05121 | Plasminogen | Anistreplase, Urokinase, Reteplase, Alteplase, | 181.88 |
| | activator inhibitor 1 | Tenecteplase, Drotrecogin alfa | |

In summary, the general approach described above may be applied to anyone of the protein-drug combinations described in Table 9 to develop a drug treatment plan or to administer the drug or drugs to the individual based on their proteomic profile (differential protein expression levels—“up” or “down” and the fold-level of that difference). Further, the approach may be used to identify proteins that may be drug targets for the treatment of individuals or groups of individuals that may share the same protein differential expression profile or profile range (i.e., have at least about a 4-fold, 5-fold, 6-fold, 7-fold, 8-fold and up to 100-fold or more in expression difference of the same protein as between tumor tissue and healthy/normal tissue).

Table 10 shows a protein expression profile generated using compositions and methods of the invention from a single patient (Subject E) with lung cancer (squamous carcinoma). By way of example, the protein Thrombospondin-2 (UniProt P35442) was found to be up-regulated in tumor tissue about 21-fold (or 21.4-fold) relative to the same protein in normal or healthy tissue from the same individual. While at this time, this protein does not have a known drug, the Thrombospondin-2 protein may be selected for drug development based on the differential expression levels between tumor tissue and healthy tissue.

By way of example, the protein Plasminogen (UniProt P00747) was found to be down-regulated in tumor tissue about 50-fold (as expressed in the table as 0.02) relative to the same protein in normal or healthy tissue from the same individual. The Plasminogen protein has known drugs that target this protein (e.g., Streptokinase, Anistreplase, Aminocaproic Acid, Urokinase, Reteplase, Alteplase, Aprotinin, Tranexamic Acid and Tenecteplase). Consequently, this individual may be responsive to a drug treatment plan that may include Streptokinase, Anistreplase, Aminocaproic Acid, Urokinase, Reteplase, Alteplase, Aprotinin, Tranexamic Acid and/or Tenecteplase. Thus, by way of example, a drug treatment plan for this individual may be developed by selecting one or more protein(s) that have differential expression between tumor tissue and healthy tissue of at least 50-fold (or at least 0.02 difference), and providing a drug treatment plan based on the drugs that target this particular protein.

By way of another example, the protein MMP-1 (UniProt P03956) was found to be up-regulated in tumor tissues relative to normal or healthy tissue by about 25-fold (or 25.28 fold). The MMP-1 protein has a known drug that targets this protein (e.g., Marimastat). Consequently, this individual may be responsive to a drug treatment plan that may include Marimastat. Thus, by way of example, a drug treatment plan for this individual may be developed by selecting one or more protein(s) that have differential expression between tumor tissue and healthy tissue of at least about 25-fold and providing a drug treatment plan based on the drugs that target this particular protein.

TABLE 10
Proteomic profile for a single individual (Subject E) based on proteins having at least
a 4-fold difference in expression between tumor tissue and normal tissue. Based on
this threshold cut-off, this individual had 128 proteins with at least a 4-fold (either
up or down) difference in tumor to healthy tissue protein expression levels.

| | | | Protein |
|---|---|---|---|
| | | | Expression |
| Uniprot | Protein Name: | Drug List: | (Tumor/Normal) |
| Q15109 | Advanced | (None found) | 0.00 |
| | glycosylation end | | |
| | product-specific | | |
| | receptor | | |
| P00747 | Plasminogen | Streptokinase, Anistreplase, Aminocaproic Acid, | 0.02 |
| | | Urokinase, Reteplase, Alteplase, Aprotinin, | |
| | | Tranexamic Acid, Tenecteplase | |
| P07451 | Carbonic | Zonisamide, Acetazolamide | 0.03 |
| | anhydrase 3 | | |
| P07483 | Fatty acid-binding | (None found) | 0.03 |
| | protein, heart RAT | | |
| P04040 | Catalase | Fomepizole | 0.04 |
| P43652 | Afamin | (None found) | 0.04 |
| P03952 | Plasma kallikrein | (None found) | 0.05 |
| | (precursor) | | 0.05 |
| P01042 | Kininogen-1, | (None found) | |
| | HMW | | |
| P00915 | Carbonic | Hydrochlorothiazide, Quinethazone, Benzthiazide, Diazoxide, | 0.05 |
| | anhydrase I | Trichlormethiazide, Methocarbamol, Amlodipine, | |
| | | Bendroflumethiazide, Brinzolamide, Dichlorphenamide, | |
| | | Methazolamide, Ethinamate, Hydroflumethiazide, | |
| | | Acetazolamide, Cyclothiazide, Zonisamide, Ethoxzolamide, | |
| | | Chlorothiazide, Methyclothiazide, Dorzolamide | |
| P00742 | Coagulation factor | Fondaparinux sodium, Menadione, Enoxaparin, Coagulation | 0.05 |
| | X (activated form) | factor VIIa, Antihemophilic Factor, Rivaroxaban, | |
| | | Apixaban, Coagulation Factor IX, Heparin | |
| P00747 | Angiostatin | Streptokinase, Anistreplase, Aminocaproic Acid, | 0.06 |
| | | Urokinase, Reteplase, Alteplase, Aprotinin, | |
| | | Tranexamic Acid, Tenecteplase | |
| P01024 | C3a anaphylatoxin | Intravenous Immunoglobulin | 0.06 |
| | des Arginine | | |
| P01019 | Angiotensinogen | (None found) | 0.06 |
| P29622 | Kallistatin | (None found) | 0.06 |
| P00742 | Coagulation | Fondaparinux sodium, Menadione, Enoxaparin, Coagulation | 0.06 |
| | Factor X | factor VIIa, Antihemophilic Factor, Rivaroxaban, | |
| | | Apixaban, Coagulation Factor IX, Heparin | |
| P01833 | Polymeric | (None found) | 0.06 |
| | immunoglobulin | | |
| | receptor | | |
| P55774 | C-C motif | (None found) | 0.07 |
| | chemokine 18 | | |
| P21810 | Biglycan | (None found) | 0.07 |
| P05543 | Thyroxine-binding | (None found) | 0.07 |
| | globulin | | |
| P05546 | Heparin cofactor 2 | Ardeparin, Sulodexide | 0.07 |
| Q14624 | Inter-alpha-trypsin | (None found) | 0.07 |
| | inhibitor heavy | | |
| | chain H4 | | |
| P01008 | Antithrombin-III | Tinzaparin, Dalteparin, Nadroparin, Fondaparinux | 0.07 |
| | | sodium, Sulodexide, Ardeparin, Enoxaparin, Heparin | |
| P02743 | Serum amyloid P- | (None found) | 0.08 |
| | component | | |
| P02775 | Neutrophil- | (None found) | 0.08 |
| | activating peptide 2 | | |
| P13686 | Tartrate-resistant | (None found) | 0.08 |
| | acid phosphatase | | |
| | type 5 | | |
| P02647 | Apolipoprotein A-I | (None found) | 0.08 |
| Q04756 | Hepatocyte growth | (None found) | 0.09 |
| | factor activator | | |
| P01042 | Kininogen-1, | (None found) | 0.09 |
| | HMW | | |
| P01031 | Complement C5 | Eculizumab, Intravenous Immunoglobulin | 0.09 |
| Q96IY4 | Carboxypeptidase | (None found) | 0.09 |
| | B2 | | |
| P02775 | Connective-tissue | (None found) | 0.09 |
| | activating peptide | | |
| | III | | |
| Q8NAC3 | Interleukin-17 | (None found) | 0.09 |
| | receptor C | | |
| Q9UGM5 | Fetuin-B | (None found) | 0.10 |
| P08603 | Complement factor | (None found) | 0.10 |
| | H | | |
| P02776 | Platelet factor 4 | Drotrecogin alfa | 0.10 |
| P02787 | Serotransferrin | Aluminium | 0.10 |
| #N/A | Complement C4-A | (None found) | 0.10 |
| | and Complement | | |
| | C4-B | | |
| P00568 | Myokinase, human | (None found) | 0.11 |
| P48061 | Stromal cell- | Tinzaparin | 0.11 |
| | derived factor 1 | | |
| P05154 | Plasma serine | Urokinase, Drotrecogin alfa | 0.11 |
| | protease inhibitor | | |
| O15467 | Small-inducible | (None found) | 0.11 |
| | cytokine A16 | | |
| P02790 | Hemopexin | (None found) | 0.11 |
| P00751 | Complement factor | (None found) | 0.11 |
| | B | | |
| P12259 | Coagulation Factor | ART-123, Drotrecogin alfa | 0.11 |
| | V | | |
| P05156 | Complement factor | (None found) | 0.11 |
| | I | | |
| P01024 | C3a anaphylatoxin | Intravenous Immunoglobulin | 0.12 |
| #N/A | Ferritin heavy and | (None found) | 0.12 |
| | light chains | | |
| P13671 | Complement | (None found) | 0.13 |
| | component C6 | | |
| #N/A | Fibrinogen alpha, | (None found) | 0.13 |
| | beta, and gamma | | |
| | chains | | |
| P07225 | Vitamin K- | Menadione, Sodium Tetradecyl Sulfate, | 0.13 |
| | dependent | Drotrecogin alfa | |
| | protein S | | |
| P01011 | Alpha-1- | (None found) | 0.13 |
| | antichymotrypsin | | |
| P02751 | Fibronectin | Ocriplasmin | 0.13 |
| P00740 | Coagulation factor | Menadione, Antihemophilic Factor | 0.13 |
| | IX | | |
| P01024 | Complement C3b, | Intravenous Immunoglobulin | 0.13 |
| | incactivated | | |
| P02748 | Complement | (None found) | 0.14 |
| | component C9 | | |
| O00585 | Small-inducible | (None found) | 0.14 |
| | cytokine A21 | | |
| P35247 | Pulmonary | (None found) | 0.15 |
| | surfactant- | | |
| | associated | | |
| | protein D | | |
| P01024 | Complement C3 | Intravenous Immunoglobulin | 0.15 |
| #N/A | C4b-A | (None found) | 0.15 |
| P00734 | Prothrombin | Ximelagatran, Menadione, Coagulation Factor | 0.15 |
| | | IX, Proflavine, Lepirudin, ART-123, | |
| | | Suramin, Bivalirudin, Argatroban, | |
| | | Dabigatran etexilate, Drotrecogin alfa | |
| P08697 | Alpha-2- | Ocriplasmin | 0.15 |
| | antiplasmin | | |
| #N/A | Hemoglobin | (None found) | 0.16 |
| P20231 | Tryptase beta-2 | (None found) | 0.16 |
| Q92563 | Testican-2 | (None found) | 0.16 |
| P04196 | Histidine-rich | (None found) | 0.16 |
| | glycoprotein | | |
| P22626 | Heterogeneous | (None found) | 0.16 |
| | nuclear | | |
| | ribonucleoproteins | | |
| | A2/B1 | | |
| P02748 | Complement | (None found) | 0.16 |
| | component C9 | | |
| P21246 | Pleiotrophin | (None found) | 0.16 |
| Q6UX06 | Olfactomedin-4 | (None found) | 0.16 |
| P62979 | Ubiquitin | (None found) | 0.16 |
| P62937 | Peptidyl-prolyl cis- | Cyclosporine, L-Proline | 0.17 |
| | trans isomerase A | | |
| Q12860 | contactin-1 | (None found) | 0.17 |
| P00740 | Coagulation factor | Menadione, Antihemophilic Factor | 0.17 |
| | IX (activated | | |
| | form) | | |
| P06681 | Complement C2 | (None found) | 0.17 |
| #N/A | Complement C5b, | (None found) | 0.17 |
| | and Complement | | |
| | component C6 | | |
| P30041 | Peroxiredoxin-6 | (None found) | 0.18 |
| P04406 | Glyceraldehyde-3- | (None found) | 0.18 |
| | phosphate | | |
| | dehydrogenase | | |
| P02775 | Neutrophil- | (None found) | 0.18 |
| | activating peptide 2 | | |
| P03951 | Coagulation factor | Coagulation Factor IX | 0.18 |
| | XI | | |
| P29401 | Transketolase | (None found) | 0.18 |
| P05186 | Alkaline | (None found) | 0.18 |
| | phosphatase, | | |
| | tissue-nonspecific | | |
| | isozyme | | |
| P04278 | Sex hormone- | (None found) | 0.18 |
| | binding globulin | | |
| Q13740 | Activated | (None found) | 0.19 |
| | leukocyte cell | | |
| | adhesion molecule | | |
| Q13219 | Pappalysin-1 | (None found) | 0.19 |
| P07585 | Decorin | (None found) | 0.20 |
| P02788 | Lactotransferrin | (None found) | 0.20 |
| Q9Y5Y7 | Lymphatic vessel | (None found) | 0.20 |
| | endothelial | | |
| | hyaluronic acid | | |
| | receptor 1 | | |
| P02751 | Fibronectin-1 | Ocriplasmin | 0.20 |
| | Fragment 3 | | |
| P16671 | Platelet | (None found) | 0.21 |
| | glycoprotein 4 | | |
| P10909 | Clusterin | (None found) | 0.21 |
| #N/A | Immunoglobulin G | (None found) | 0.21 |
| P30086 | prostatic binding | (None found) | 0.22 |
| | protein | | |
| P27918 | Properdin | (None found) | 0.22 |
| P02649 | Apolipoprotein E | Serum albumin iodonated, Human Serum Albumin | 0.22 |
| | (isoform E3) | | |
| P09769 | Proto-oncogene | (None found) | 0.22 |
| | tyrosine-protein | | |
| | kinase FGR | | |
| P07195 | L-lactate | (None found) | 0.23 |
| | dehydrogenase B | | |
| | chain | | |
| P09038 | Heparin-binding | Pentosan Polysulfate, Sucralfate, Sirolimus | 0.23 |
| | growth factor 2 | | |
| P16109 | P-selectin | Dalteparin, Nadroparin, Heparin | 0.24 |
| P03950 | Angiogenin | (None found) | 0.24 |
| #N/A | Immunoglobulin M | (None found) | 0.24 |
| P02649 | Apolipoprotein E | Serum albumin iodonated, Human Serum Albumin | 0.24 |
| | (isoform E2) | | |
| P10643 | Complement | (None found) | 0.25 |
| | component C7 | | |
| P42684 | Tyrosine-protein | Dasatinib, Adenosine triphosphate | 4.02 |
| | kinase ABL2 | | |
| #N/A | #N/A | (None found) | 4.03 |
| P09238 | Stromelysin-2 | Marimastat | 4.05 |
| O00541 | Pescadillo homolog | (None found) | 4.10 |
| | 1 | | |
| Q6UXX9 | R-spondin-2 | (None found) | 4.13 |
| P04818 | TS | Pemetrexed, Trimethoprim, Fluorouracil, Leucovorin, | 4.60 |
| | | Gemcitabine, Pralatrexate, Capecitabine, Raltitrexed, | |
| | | Trifluridine, Floxuridine | |
| P51671 | Eotaxin | (None found) | 4.85 |
| P41743 | Protein kinase C | (None found) | 4.86 |
| | iota type | | |
| O00339 | Matrilin-2 | (None found) | 5.08 |
| Q9H773 | XTP3- | (None found) | 5.46 |
| | transactivated gene | | |
| | A protein | | |
| O43291 | Kunitz-type | (None found) | 5.47 |
| | protease inhibitor 2 | | |
| #N/A | Cell division | (None found) | 5.68 |
| | control protein 2 | | |
| | homolog, | | |
| | G2/mitotic-specific | | |
| | cyclin-B1 Complex | | |
| Q99706 | Killer cell | (None found) | 5.79 |
| | immunoglobulin- | | |
| | like receptor 2DL4 | | |
| P10145 | Interleukin-8 | (None found) | 5.85 |
| P02735 | Serum amyloid A | (None found) | 6.01 |
| | protein | | |
| O43278 | Kunitz-type | (None found) | 6.23 |
| | protease inhibitor 1 | | |
| O60911 | Cathepsin L2 | (None found) | 6.44 |
| Q7LFX5 | N- | (None found) | 6.81 |
| | acetylgalactosamine | | |
| | 4-sulfate 6-O- | | |
| | sulfotransferase | | |
| P52823 | Stanniocalcin-1 | (None found) | 7.26 |
| P21741 | Midkine | (None found) | 10.06 |
| P00749 | Urokinase-type | Urokinase, Amiloride | 10.55 |
| | plasminogen | | |
| | activator | | |
| P19957 | Elafin | (None found) | 13.61 |
| P18669 | Phosphoglycerate | (None found) | 17.51 |
| | mutase 1 | | |
| P35442 | Thrombospondin-2 | (None found) | 21.40 |
| P03956 | MMP-1 | Marimastat | 25.28 |
| P39900 | Macrophage | Acetohydroxamic Acid, Marimastat | 30.88 |
| | metalloelastase | | |

In summary, the general approach described above may be applied to anyone of the protein-drug combinations described in Table 10 to develop a drug treatment plan or to administer the drug or drugs to the individual based on their proteomic profile (differential protein expression levels—“up” or “down” and the fold-level of that difference). Further, the approach may be used to identify proteins that may be drug targets for the treatment of individuals or groups of individuals that may share the same protein differential expression profile or profile range (i.e., have at least about a 4-fold, 5-fold, 6-fold, 7-fold, 8-fold and up to 100-fold or more in expression difference of the same protein as between tumor tissue and healthy/normal tissue).

Table 11 shows exemplary protein and drugs that target the listed proteins.

TABLE 11

| UniProt | Protein Name | Known Drugs |
|---|---|---|
| P01023 | α2-Macroglobulin | Ocriplasmin, Bacitracin, Becaplermin |
| P00519 | c-abl oncogene 1, non-receptor | Dasatinib, Bosutinib, Adenosine triphosphate, |
| | tyrosine kinase | Nilotinib, Ponatinib, Imatinib, Regorafenib |
| P42684 | v-abl Abelson murine leukemia viral | Dasatinib, Adenosine triphosphate |
| | oncogene homolog 2 | |
| P42684 | v-abl Abelson murine leukemia viral | Dasatinib, Adenosine triphosphate |
| | oncogene homolog 2 | |
| P16112 | Aggrecan core protein | (None found) |
| Q9BYF1 | Angiotensin-converting enzyme 2 | Moexipril, Lisinopril |
| P24666 | Acid phosphatase 1, soluble | Adenine |
| P13686 | Tartrate-resistant acid phosphatase | |
| | type 5 | |
| P36896 | Activin Serine-threonine-protein | Adenosine triphosphate |
| | kinase receptor type-1B | |
| P37023 | Activin receptor-like kinase 1 | Adenosine triphosphate |
| Q03154 | Aminoacylase-1 | L-Aspartic Acid |
| O43184 | ADAM metallopeptidase domain 12 | |
| Q13443 | ADAM metallopeptidase domain 9 | |
| Q9UHI8 | ADAM metalloproteinase with | |
| | thrombospondin motifs 1 | |
| Q76LX8 | ADAM metallopeptidase with | |
| | thrombospondin motifs 13 | |
| Q8TE58 | ADAM metallopeptidase with | |
| | thrombospondin motifs 15 | |
| O75173 | ADAM metallopeptidase with | |
| | thrombospondin motifs | |
| | 4/Aggrecanase 1 | |
| Q9UNA0 | ADAM metallopeptidase with | |
| | thrombospondin motifs | |
| | 5/Aggrecanase 2 | |
| P18509 | Pituitary adenylate cyclase- | |
| | activating polypeptide 27 | |
| P18509 | Pituitary adenylate cyclase- | |
| | activating polypeptide 38 | |
| Q15848 | Adiponectin | |
| P25098 | β-adrenergic receptor kinase 1 | Adenosine triphosphate |
| P30566 | PUR8/Adenylosuccinate lyase | |
| P43652 | Afamin | |
| Q15109 | RAGE, soluble/Advanced glycosylation | |
| | end product-specific receptor | |
| O95994 | Anterior gradient protein 2 homolog | |
| O00253 | Agouti-related protein | |
| P01019 | Angiotensinogen | |
| P02765 | α2-HS-Glycoprotein | |
| P55008 | Allograft inflammatory factor 1 | |
| Q12904 | Endothelial-Monocyte Activating | |
| | Polypeptide 2 | |
| O00170 | AH receptor-interacting protein | |
| P00568 | Adenylate Kinase 1 | |
| P14550 | Alcohol dehydrogenase (NADP+)/Ado- | |
| | keto reductase family 1 member A1 | |
| O43488 | Aflatoxin B1 aldehyde reductase | |
| P02768 | Albumin | |
| Q13740 | Activated leukocyte cell adhesion | |
| | molecule | |
| P05186 | Alkaline phosphatase, tissue- | |
| | nonspecific isozyme | |
| P03971 | Muellerian-inhibiting factor | |
| Q16671 | Anti-Mullerian hormone receptor, | Adenosine triphosphate |
| | type II | |
| Q86YT9 | Junctional adhesion molecule-like | |
| Q9BXJ7 | Amnionless | Hydroxocobalamin |
| P03950 | Angiogenin | |
| Q15389 | Angiopoietin-1 | |
| O15123 | Angiopoietin-2 | |
| Q9Y264 | Angiopoietin-4 | |
| Q9Y5C1 | Angiopoietin-related 3 | |
| Q9BY76 | Angiopoietin-related 4 | |
| Q92688 | Acidic leucine-rich nuclear | |
| | phosphoprotein 32 family member B | |
| P04083 | Annexin A1 | Hydrocortisone, Dexamethasone, Amcinonide |
| P07355 | Annexin A2 | Tenecteplase |
| P08133 | Annexin A6 | |
| P02743 | Serum amyloid P | |
| P02647 | Apolipoprotein A-I | |
| P04114 | Apolipoprotein B | |
| P05090 | Apolipoprotein D | |
| P02649 | Apolipoprotein E | Serum albumin iodonated, Human Serum Albumin |
| P02649 | Apolipoprotein E3 | Serum albumin iodonated, Human Serum Albumin |
| P02649 | Apolipoprotein E4 | Serum albumin iodonated, Human Serum Albumin |
| P02649 | Apolipoprotein E (isoform E2) | Serum albumin iodonated, Human Serum Albumin |
| P05067 | Amyloid β A4 protein | |
| P15514 | Amphiregulin | |
| P05089 | Arginase-1 | L-Ornithine |
| Q99856 | ARID domain-containing protein 3A | |
| P56211 | cAMP-regulated phosphoprotein 19 | |
| P15289 | Arylsulfatase A | |
| P15848 | Arylsulfatase B | |
| Q5T4W7 | Artemin | |
| Q9NR71 | Neutral ceramidase | |
| P07306 | Asialoglycoprotein receptor 1 | |
| P06576 | ATP synthase β-subunit, mitochondrial | |
| O14965 | Aurora kinase A | |
| Q96GD4 | Aurora-related kinase 2 | |
| P20160 | Azurocidin | |
| P61769 | β2-Microglobulin | |
| P50895 | Basal Cell Adhesion Molecule | |
| Q96GW7 | Brevican | |
| O75815 | BCAR3 breast cancer anti-estrogen | |
| | resistance 3 | |
| P10415 | Apoptosis regulator Bcl-2 | Rasagiline, Paclitaxel, Ibuprofen, Docetaxel |
| Q16548 | Bcl-2-related protein A1 | |
| Q07817 | Apoptosis regulator Bcl-X | |
| P23560 | Brain-derived neurotrophic factor | |
| P21810 | Biglycan | |
| Q13489 | Apoptosis inhibitor 2/C-IAP2 | |
| O15392 | Survivin | |
| Q96CA5 | Livin/baculoviral IAP repeat containing 7 | |
| P13497 | Bone morphogenetic protein-1 | |
| O95393 | Bone morphogenetic protein-10 | |
| P22004 | Bone morphogenetic protein-6 | |
| P18075 | Bone morphogenetic protein-7 | |
| Q8N8U9 | Bone morphogenetic protein-binding | |
| | endothelial regulator protein | |
| P36894 | Bone morphogenetic protein receptor | |
| | type IA | |
| Q13873 | Bone morphogenetic protein type II | |
| | receptor | |
| P51813 | Tyrosine kinase Etk | |
| Q9BWV1 | Shh receptor Boc | |
| P17213 | Bactericidal permeability-increasing | |
| | protein | |
| Q92994 | BRF1 | |
| P35613 | Extracellular matrix | |
| | metalloproteinase inducer | |
| Q10588 | Bone marrow stromal cell | |
| | antigen/CD157 | |
| Q06187 | Tyrosine kinase Bruton | |
| P02745 | Complement C1q | |
| P02746 | | |
| P02747 | | |
| Q07021 | Complement C1q subcomponent- | |
| | binding protein, mitochondrial | |
| P00736 | Complement C1r | Alemtuzumab, Daclizumab, Ibritumomab, Trastuzumab, |
| | | Bevacizumab, Efalizumab, Muromonab, Adalimumab, |
| | | Palivizumab, Abciximab, Natalizumab, Basiliximab, |
| | | Cetuximab, Rituximab, Gemtuzumab ozogamicin, |
| | | Etanercept, Tositumomab, Alefacept |
| P09871 | Complement C1s | Adalimumab, Abciximab, Basiliximab, Cetuximab, |
| | | Ibritumomab, Rituximab, Gemtuzumab ozogamicin, |
| | | Etanercept, Trastuzumab, Muromonab |
| P06681 | Complement C2 | |
| P01024 | Complement C3b, inactivated | Intravenous Immunoglobulin |
| P01024 | Complement C3 | Intravenous Immunoglobulin |
| P01024 | Complement C3a anaphylatoxin | Intravenous Immunoglobulin |
| | des Arginine | |
| P01024 | Complement C3b | Intravenous Immunoglobulin |
| P01024 | Complement C3d | Intravenous Immunoglobulin |
| P01024 | Complement C3a anaphylatoxin | Intravenous Immunoglobulin |
| P0C0L4 | Complement C4b | |
| P0C0L5 | | |
| P0C0L4, | Complement C4 | |
| P0C0L5 | | |
| P01031 | Complement C5 | Eculizumab, Intravenous Immunoglobulin |
| P01031 | Complement C5a | Eculizumab, Intravenous Immunoglobulin |
| P01031, | Complement C5b, 6 Complex | |
| P13671 | | |
| P13671 | Complement C6 | |
| P10643 | Complement C7 | |
| P07357, | Complement C8 | |
| P07358, | | |
| P07360 | | |
| P02748 | Complement C9 | |
| P02748 | Complement C9 | |
| P00915 | Carbonic anhydrase I | Hydrochlorothiazide, Quinethazone, Benzthiazide, |
| | | Diazoxide, Trichlormethiazide, Methocarbamol, Amlodipine, |
| | | Bendroflumethiazide, Brinzolamide, Dichlorphenamide, |
| | | Methazolamide, Ethinamate, Hydroflumethiazide, |
| | | Acetazolamide, Cyclothiazide, Zonisamide, Ethoxzolamide, |
| | | Chlorothiazide, Methyclothiazide, Dorzolamide |
| Q9NS85 | Carbonic anhydrase- | Zonisamide |
| | related protein X | |
| Q8N1Q1 | Carbonic anhydrase XIII | Zonisamide |
| P00918 | Carbonic anhydrase II | Hydrochlorothiazide, Benzthiazide, Bendroflumethiazide, |
| | | Zonisamide, Topiramate, Methyclothiazide, Quinethazone, |
| | | Furosemide, Acetazolamide, Ethoxzolamide, Diazoxide, |
| | | Dichlorphenamide, Ethinamate, Cyclothiazide, Dorzolamide, |
| | | Trichlormethiazide, Brinzolamide, Methazolamide, |
| | | Hydroflumethiazide, Chlorothiazide |
| P07451 | Carbonic anhydrase III | Zonisamide, Acetazolamide |
| P22748 | Carbonic anhydrase IV | Hydrochlorothiazide, Benzthiazide, Trichlormethiazide, |
| | | Bendroflumethiazide, Brinzolamide, Dichlorphenamide, |
| | | Methazolamide, Hydroflumethiazide, Acetazolamide, |
| | | Cyclothiazide, Zonisamide, Ethoxzolamide, Chlorothiazide, |
| | | Topiramate, Methyclothiazide, Dorzolamide |
| P23280 | Carbonic anhydrase VI | Zonisamide |
| P43166 | Carbonic anhydrase VII | Dichlorphenamide, Zonisamide, Methazolamide, |
| | | Acetazolamide, Ethoxzolamide |
| Q16790 | Carbonic anhydrase IX | Zonisamide, Hydrochlorothiazide, Hydroflumethiazide, |
| | | Benzthiazide |
| Q9BY67 | Nectin-like protein 2 | |
| Q8N126 | Nectin-like protein 1 | |
| P27797 | Calreticulin | Melatonin, Antihemophilic Factor, Tenecteplase |
| Q14012 | Calcium-calmodulin-dependent | |
| | protein kinase I | |
| Q8IU85 | Calcium-calmodulin-dependent | |
| | protein kinase ID | |
| Q9UQM7 | Calcium-calmodulin-dependent | |
| | protein kinase II α | |
| Q13554 | Calcium-calmodulin-dependent | |
| | protein kinase II β | |
| Q13557 | Calcium-calmodulin-dependent | |
| | protein kinase II δ | |
| Q8N5S9 | Calcium-calmodulin-dependent | |
| | protein kinase kinase 1, α | |
| P40121 | Macrophage-capping protein | |
| P07384 | Calpain 1 | |
| P04632 | | |
| Q92851 | Caspase 10, apoptosis-related | |
| | cysteine peptidase | |
| P42575 | Caspase-2 | |
| P42574 | Caspase-3 | Minocycline |
| P20810 | Calpastatin | |
| P04040 | Catalase | Fomepizole |
| P45973 | Chromobox protein homolog 5 | |
| Q76M96 | Coiled-coil domain-containing | |
| | protein 80 | |
| P22362 | CC chemokine I-309/CCL1 | |
| P51671 | Eotaxin/CCL11 | |
| Q99616 | Monocyte chemoattractant protein 4 | |
| Q16627 | Hemofiltrate CC Chemokine 1/CCL14 | |
| Q16663 | Macrophage inflammatory protein | |
| | 5/CCL15 | |
| O15467 | Liver-expressed chemokine/CCL16 | |
| Q92583 | Thymus and activation-regulated | |
| | chemokine/CCL17 | |
| P55774 | Macrophage inflammatory protein | |
| | 4/Pulmonary and activation-regulated | |
| | chemokine/CCL18 | |
| Q99731 | Macrophage inflammatory protein 3 | |
| | β/CCL19 | |
| P13500 | Monocyte chemoattractant protein 1 | Mimosine, Danazol |
| P78556 | Macrophage inflammatory protein 3 | |
| | α/CCL20 | |
| O00585 | 6Ckine/CCL21 | |
| O00626 | Macrophage-derived chemokine | |
| P55773 | Myeloid progenitor inhibitory factor | |
| | 1/CCL23 | |
| P55773 | Ck-β-8-1/Macrophage inflammatory | |
| | protein 3 splice variant (aa 46-137) | |
| O00175 | Eotaxin-2 | |
| O15444 | Thymus expressed chemokine/CCL25 | |
| Q9Y4X3 | Cutaneous T-cell-attracting | |
| | chemokine/CCL27 | |
| Q9NRJ3 | CCL28 | |
| P10147 | Macrophage inflammatory protein 1- | |
| | α/CCL3 | |
| P16619 | LD78-β/CCL3L1 | |
| Q8NHW4 | Lymphocyte Activation Gene-1/CCL4L1 | |
| P13501 | RANTES/CCL5 | |
| P80098 | Monocyte chemoattractant protein 3 | |
| P80075 | Monocyte chemoattractant protein 2 | |
| P14635 | Cyclin B1 | |
| Q6YHK3 | CD109 | |
| Q86VB7 | Scavenger receptor cysteine-rich type 1 | WF10 |
| | protein M130 chain/Soluble CD163 | |
| P41217 | CD200/OX-2 membrane glycoprotein | |
| Q8TD46 | CD200 receptor 1 | |
| Q9UJ71 | Langerin | |
| Q9NNX6 | Dendritic cell-specific ICAM-3-grabbing | |
| | nonintegrin 1/CD209 | |
| P20273 | CD22 | |
| Q15762 | DNAX accessory molecule 1/CD226 | |
| P26842 | CD27/TNFRSF7 | |
| Q9NZQ7 | B7 homolog 1/CD274 | |
| Q08708 | CMRF35-like molecule 6/CD300c | |
| P20138 | Siglec-3 | Gemtuzumab ozogamicin |
| P16671 | CD36 ANTIGEN | |
| P01730 | T-cell surface glycoprotein CD4 | Antithymocyte globulin |
| P29965 | CD40 ligand | |
| P09326 | CD48 | |
| P08174 | CD55/Complement decay-accelerating | Chloramphenicol |
| | factor/DAF | |
| O43866 | CD5 antigen-like | |
| P32970 | CD70 | |
| P33681 | T-lymphocyte activation antigen CD80 | Belatacept, Abatacept |
| Q9UIB8 | Signaling lymphocytic activation | |
| | molecule 5 | |
| P42081 | B7-2/CD86 | Belatacept, Abatacept, Antithymocyte globulin |
| P48960 | CD97 | |
| P06493 | Cyclin-dependent kinase 1: cyclin B | |
| P14635 | complex | |
| Q16543 | Hsp90 co-chaperone Cdc37 | |
| Q9Y5S2 | Myotonic dystrophy protein | |
| | kinase-like β | |
| P12830 | Cadherin-1 | |
| P55289 | Cadherin-12 | |
| P55291 | Cadherin 15, type 1, M-cadherin | |
| | (myotubule) | |
| P19022 | Cadherin 2, type 1, N-cadherin | |
| | (neuronal) | |
| P22223 | Cadherin-3 | |
| P33151 | Cadherin-5 | Lenalidomide |
| P55285 | Cadherin-6 | |
| P24941 | Cyclin-dependent kinase 2: cyclin A | |
| P20248 | complex | |
| Q00535 | Cyclin-dependent kinase 5: activator p35 | |
| Q15078 | complex | |
| P49336 | Cyclin-dependent kinase 8: cyclin C | |
| P24863 | complex | |
| P46527 | Cyclin-dependent kinase inhibitor p27 | |
| Q49AH0 | Conserved dopamine neurotrophic | |
| | factor | |
| Q4KMG0 | Cell adhesion molecule-related down- | |
| | regulated by oncogenes | |
| P00751 | Complement factor B | |
| P0CG37 | Cryptic protein | |
| P00746 | Complement factor D | |
| P08603 | Complement factor H | |
| Q9BXR6 | Complement factor H-related 5 | |
| P05156 | Complement factor I | |
| P23528 | Cofilin-1 | |
| P27918 | Properdin | |
| P01215, | Human Chorionic Gonadotropin | |
| P01233 | | |
| P01215, | Follicle stimulating hormone | |
| P01225 | | |
| P01215, | Luteinizing hormone | |
| P01229 | | |
| P01215 | Thyroid Stimulating Hormone | |
| P01222 | | |
| O14757 | Serine-threonine-protein kinase Chk1 | |
| O96017 | Serine-threonine-protein kinase Chk2 | |
| Q13231 | Chitotriosidase-1 | |
| O00533 | Neural cell adhesion molecule L1-like | |
| | protein | |
| Q9BU40 | Chordin-Like 1 | |
| Q7LFX5 | Carbohydrate sulfotransferase 15 | |
| Q9Y4C5 | Carbohydrate sulfotransferase 2 | |
| Q9GZX3 | Carbohydrate sulfotransferase 6 | |
| Q8WWK9 | CKAP2/Cytoskeleton-associated | |
| | protein 2 | |
| P12277 | Creatine kinase-BB | Creatine |
| P12277 | Creatine kinase-MB | |
| P06732 | | |
| P06732 | Creatine kinase-MM | Creatine |
| Q9Y240 | Stem Cell Growth Factor | |
| Q9Y240 | Stem Cell Growth Factor | |
| Q9P126 | C-type lectin domain family 1 member B | |
| Q9H2X3 | Dendritic cell-specific ICAM-3-grabbing | |
| | nonintegrin 2/CD299 | |
| Q9BXN2 | Dectin-1 | |
| O00299 | Nuclear chloride ion channel 27 | |
| P10909 | Clusterin | |
| P23946 | Chymase | |
| P30085 | UMP-CMP kinase | Gemcitabine |
| Q96KN2 | Carnosine dipeptidase 1 | |
| Q96KP4 | Glutamate carboxypeptidase | |
| P26441 | Ciliary Neurotrophic Factor | |
| P26992 | Ciliary neurotrophic factor receptor α | |
| Q12860 | Contactin-1 | |
| Q02246 | Contactin-2 | |
| Q8IWV2 | Contactin-4 | |
| O94779 | Contactin-5 | |
| P39060 | Endostatin | |
| Q86Y22 | Collagen α-1(XXIII) chain | |
| P27658 | Collagen α-1(VIII) chain | |
| Q9BWP8 | Collectin Kidney 1 | |
| Q5KU26 | Collectin placenta 1 | |
| Q86VX2 | COMM domain containing 7 | |
| Q14019 | Coactosin-like protein 1 | |
| Q96IY4 | Thrombin-Activatable Fibrinolysis | |
| | Inhibitor | |
| P16870 | Carboxypeptidase E | Insulin, Insulin Regular |
| Q99829 | Copine-1 | |
| P54108 | Cysteine-rich secretory protein 3 | |
| P46108 | Adaptor protein Crk-I | |
| O75462 | Cytokine receptor-like factor | |
| Q9UBD9 | 1: Cardiotrophin-like cytokine factor 1 | |
| | Complex | |
| Q9HC73 | Thymic stromal lymphopoietin protein | |
| | receptor | |
| P02741 | C-reactive protein | inhaled insulin |
| O95727 | CRTAM/cytotoxic and regulatory T cell | |
| | molecule | |
| P09603 | Macrophage colony-stimulating factor 1 | |
| P07333 | Macrophage colony-stimulating factor 1 | Sunitinib, Imatinib |
| | receptor | |
| P04141 | Granulocyte-macrophage colony- | |
| | stimulating factor | |
| P09919 | Granulocyte colony-stimulating factor | |
| Q99062 | Granulocyte colony-stimulating factor | Pegfilgrastim, Filgrastim |
| | receptor | |
| P41240 | C-Src kinase | |
| P47710 | α-S1-casein | |
| P68400 | Casein kinase II subunit α | |
| P68400 | Casein kinase II subunit α | |
| P68400 | Casein kinase II α1: β heterodimer | |
| P67870 | | |
| P19784 | Casein kinase II α2: β heterodimer | |
| P67870 | | |
| P01037 | Cystatin SN | |
| P09228 | Cystatin SA | |
| P01034 | Cystatin C | |
| P01036 | Cystatin S | |
| P28325 | Cystatin D | |
| Q15828 | Cystatin M | |
| O76096 | Cystatin F | |
| Q16619 | Cardiotrophin-1 | |
| P29279 | Connective tissue growth factor | |
| P16410 | Cytotoxic T-lymphocyte-4 | Ipilimumab |
| P10619 | Cathepsin A | |
| P07858 | Cathepsin B | |
| P53634 | Cathepsin C | |
| P07339 | Cathepsin D | Insulin, Insulin Regular |
| P14091 | Cathepsin E | |
| P08311 | Cathepsin G | |
| P09668 | Cathepsin H | |
| O60911 | Cathepsin V | |
| P25774 | Cathepsin S | |
| Q9UBR2 | Cathepsin Z | |
| P78423 | Fractalkine/CX3CL-1 | |
| P09341 | Gro-α | |
| P02778 | Interferon-γ induced protein | |
| O14625 | Interferon-γ-inducible protein-9 | |
| P48061 | Stromal cell-derived factor 1 | Tinzaparin |
| P48061 | Stromal cell-derived factor 1 | Tinzaparin |
| O43927 | B lymphocyte chemoattractant/CXCL13 | |
| Q9H2A7 | Scavenger receptor for | |
| | phosphatidylserine and oxidized low | |
| | density lipoprotein/CXCL16 | |
| P19876 | Gro-γ/β | |
| P19875 | | |
| P19876 | Gro-γ/β | |
| P19875 | | |
| P42830 | Epithelial-derived neutrophil-activating | |
| | protein 78/CXCL5 | |
| P80162 | Granulocyte chemotactic protein | |
| | 2/CXCL6 | |
| P99999 | Cytochrome c | Minocycline |
| P08684 | Cytochrome P450 3A4 | Paliperidone |
| Q9UIK4 | Death-associated protein kinase 2 | |
| Q9UJU6 | Drebrin-like HIP-55 | |
| P07585 | Bone proteoglycan II | |
| Q13561 | Dynactin subunit 2 | |
| Q9H773 | dCTP pyrophosphatase 1 | |
| P20711 | Dopa decarboxylase | Carbidopa |
| Q08345 | Discoidin domain receptor 1 | Imatinib |
| Q16832 | Discoidin domain receptor 2 | Regorafenib |
| Q9UMR2 | DEAD box RNA helicase 19B | |
| O43323 | Desert Hedgehog N-Terminus | |
| Q9NR28 | Diablo, IAP-binding mitochondrial | |
| | protein | |
| O94907 | Dickkopf-related protein 1 | |
| Q9UBP4 | Dickkopf-related protein 3 | |
| Q9UBT3 | Dickkopf-related protein 4 | |
| Q9UK85 | Soggy-1 | |
| O00548 | Delta-like protein 1 (DLL1) | |
| Q9NR61 | Drosophila Delta homolog 4 | |
| Q13316 | Dentin matrix protein 1 | |
| P25685 | Hsp40 | |
| Q96DA6 | DnaJ homolog | |
| Q9UHL4 | Dipeptidyl-peptidase II | |
| Q07507 | Dermatopontin | |
| Q14574 | Desmocollin-3 | |
| Q02413 | Desmoglein-1 | |
| Q14126 | Desmoglein-2 | |
| P51452 | Vaccinia Virus VH1-related | |
| | Phosphatase/Dual specificity protein | |
| | phosphatase 3 | |
| P63167 | Dynein light chain 1 | |
| Q9NP97 | Dynein light chain roadblock-type 1 | |
| O43781 | Dual-specificity protein kinase 3 | |
| P42892 | Endothelin-converting enzyme 1 | |
| Q16610 | Extracellular matrix protein-1 | |
| Q92838 | Ectodysplasin-A, secreted form | |
| Q9HAV5 | X-linked ectodysplasin-A2 receptor | |
| Q9UNE0 | Ectodermal Dysplasia Receptor | |
| P24534 | Elongation factor 1-β | |
| P52798 | Ephrin-A4 | |
| P52803 | Ephrin-A5 | |
| Q15768 | Ephrin-B3 | |
| P00533 | erbB1/HER1 | Trastuzumab, Lidocaine, Lapatinib, Afatinib, |
| | | Panitumumab, Gefitinib, Cetuximab, Erlotinib, Vandetanib |
| Q96KQ7 | Histone H3-K9 methyltransferase 3 | |
| P38919 | Eukaryotic translation initiation factor | |
| | 4A-III | |
| Q13542 | Eukaryotic translation initiation factor | |
| | 4E-binding protein 2 | |
| P78344 | Eukaryotic translation initiation factor | |
| | G2 | |
| P55010 | Eukaryotic translation initiation factor 5 | |
| P63241 | Eukaryotic translation initiation factor | |
| | 5A | |
| P08246 | Neutrophil elastase | Pegfilgrastim, Filgrastim, Alpha-1-proteinase inhibitor |
| Q9UHX3 | EGF-like module-containing mucin-like | |
| | receptor 2 | |
| P17813 | Endoglin | |
| Q6UWV6 | Alkaline Sphingomyelinase | |
| P49961 | CD39 | |
| O75355 | Ectonucleoside triphosphate | |
| | diphosphohydrolase 3/CD39L3 | |
| O75356 | Ectonucleoside triphosphate | |
| | diphosphohydrolase 5/CD39L4 | |
| P11171 | erythrocyte membrane protein 4.1 | |
| P21709 | Ephrin type-A receptor 1 | |
| Q5JZY3 | EPH receptor A10 | |
| P29317 | Ephrin type-A receptor 2 | Dasatinib, Regorafenib |
| P29320 | Ephrin type-A receptor 3 | |
| P54756 | Ephrin type-A receptor 5 | |
| P29323 | EPH receptor B2 | |
| P54760 | Ephrin type-B receptor 4 | |
| O15197 | EPH receptor B6 | |
| P01588 | Erythropoietin | |
| P19235 | Erythropoietin receptor | Darbepoetin alfa, Epoetin alfa, Epoetin |
| | | Zeta, Peginesatide |
| Q9UBC2 | Epidermal growth factor receptor | |
| | substrate 15-like 1 | |
| Q9NZ08 | Endoplasmic reticulum aminopeptidase | |
| | 1 | |
| P04626 | erbB2/HER2 | Lapatinib, Afatinib, Trastuzumab, Pertuzumab, ado- |
| | | trastuzumab emtansine |
| P21860 | erbB3/HER3 | |
| Q15303 | erbB4/HER4 | Afatinib |
| O14944 | Epiregulin | |
| P30040 | Endoplasmic reticulum resident protein | |
| | 29 | |
| Q96AP7 | Endothelial cell-selective adhesion | |
| | molecule | |
| P10768 | Esterase D | Glutathione |
| Q9NQ30 | Endocan | |
| P03372 | Estrogen receptor | Estriol, Allylestrenol, Norgestimate, Ethynodiol, |
| | | Tamoxifen, Quinestrol, Levonorgestrel, Medroxyprogesterone |
| | | Acetate, Chlorotrianisene, Diethylstilbestrol, Dienestrol, |
| | | Progesterone, Toremifene, Ethinyl Estradiol, Desogestrel, |
| | | Estradiol, Ospemifene, Melatonin, Clomifene, Fluoxymesterone, |
| | | Danazol, Estrone, Naloxone, Raloxifene, Estramustine, |
| | | Estropipate, Etonogestrel, Trilostane, Fulvestrant, |
| | | Conjugated Estrogens, Mestranol |
| O95571 | Ethylmalonic encephalopathy 1 | |
| P00742 | Coagulation Factor Xa | Fondaparinux sodium, Menadione, Enoxaparin, Coagulation |
| | | factor VIIa, Antihemophilic Factor, Rivaroxaban, |
| | | Apixaban, Coagulation Factor IX, Heparin |
| P00742 | Coagulation Factor X | Fondaparinux sodium, Menadione, Enoxaparin, Coagulation |
| | | factor VIIa, Antihemophilic Factor, Rivaroxaban, |
| | | Apixaban, Coagulation Factor IX, Heparin |
| P03951 | Coagulation Factor XI | Coagulation Factor IX |
| P00734 | Thrombin | Ximelagatran, Menadione, Coagulation Factor |
| | | IX, Proflavine, Lepirudin, ART-123, |
| | | Suramin, Bivalirudin, Argatroban, Dabigatran |
| | | etexilate, Drotrecogin alfa |
| P00734 | Prothrombin | Ximelagatran, Menadione, Coagulation Factor |
| | | IX, Proflavine, Lepirudin, ART-123, |
| | | Suramin, Bivalirudin, Argatroban, Dabigatran |
| | | etexilate, Drotrecogin alfa |
| P13726 | Tissue Factor | Coagulation factor VIIa |
| P12259 | Coagulation Factor V | ART-123, Drotrecogin alfa |
| P08709 | Coagulation Factor VII | Coagulation factor VIIa, Menadione, Coagulation Factor |
| | | IX |
| P00740 | Coagulation factor IX | Menadione, Antihemophilic Factor |
| P00740 | Coagulation Factor IXab | Menadione, Antihemophilic Factor |
| P05413 | Fatty acid binding protein, heart-type | |
| P07483 | 0 | |
| Q01469 | Fatty acid binding protein, epidermal- | |
| | type | |
| O95990 | Down-regulated in renal cell carcinoma | |
| | 1 | |
| Q9H098 | Protein FAM107B | |
| Q12884 | Fibroblast activation protein α | |
| P48023 | Fas ligand | |
| P24071 | Immunoglobulin A Fc receptor | |
| P06734 | CD23 | |
| P12314 | High affinity Immunoglobulin G Fc | Alemtuzumab, Daclizumab, Ibritumomab, Trastuzumab, |
| | receptor I | Bevacizumab, Efalizumab, Muromonab, Adalimumab, |
| | | Palivizumab, Abciximab, Natalizumab, Intravenous |
| | | Immunoglobulin, Basiliximab, Cetuximab, Rituximab, |
| | | Gemtuzumab ozogamicin, Etanercept, Tositumomab, |
| | | Alefacept, Porfimer, Methyl aminolevulinate |
| P12318 | Low affinity immunoglobulin gamma Fc | |
| P31994 | region receptor II-a/b | |
| P12318 | Low affinity immunoglobulin gamma Fc | |
| P31994 | region receptor II-a/b | |
| O75015 | Immunoglobulin G Fc region receptor | Alemtuzumab, Daclizumab, Ibritumomab, Trastuzumab, |
| | III-B, low affinity | Bevacizumab, Efalizumab, Muromonab, Adalimumab, |
| | | Palivizumab, Abciximab, Natalizumab, Intravenous |
| | | Immunoglobulin, Basiliximab, Cetuximab, Rituximab, |
| | | Gemtuzumab ozogamicin, Etanercept, Tositumomab, |
| | | Alefacept |
| O00602 | Ficolin-1 | |
| Q15485 | Ficolin-2 | |
| O75636 | Ficolin-3 | |
| Q96P31 | Fc receptor-like protein 3 | |
| P16591 | Tyrosine kinase Fer | |
| Q9UGM5 | Fetuin B | |
| P02671 | Fibrinogen | |
| P02675 | | |
| P02679 | | |
| P02671 | D-dimer | |
| P02675 | | |
| P02679 | | |
| P05230 | Acidic fibroblast growth | Pazopanib, Amlexanox, Pentosan Polysulfate |
| | factor/endothelial cell growth factor | |
| O15520 | Fibroblast growth factor | |
| | 10/Keratinocyte growth factor 2 | |
| P61328 | Fibroblast growth factor 12 | |
| O43320 | Fibroblast growth factor 16 | |
| O60258 | Fibroblast growth factor 17 | |
| O76093 | Fibroblast growth factor 18 | |
| O95750 | Fibroblast growth factor 19 | |
| P09038 | Basic fibroblast growth factor | Pentosan Polysulfate, Sucralfate, Sirolimus |
| Q9NP95 | Fibroblast growth factor 20 | |
| Q9GZV9 | Fibroblast growth factor 23 | |
| P08620 | Fibroblast growth factor 4 | Pentosan Polysulfate |
| P12034 | Fibroblast growth factor 5 | |
| P10767 | Fibroblast growth factor 6 | |
| P21781 | Fibroblast growth factor 7 | |
| P55075 | Fibroblast growth factor 8 isoform B | |
| P55075 | Fibroblast growth factor 8 isoform A | |
| P31371 | Fibroblast growth factor 9 | |
| P11362 | Basic fibroblast growth factor receptor 1 | Palifermin, Sorafenib, Ponatinib, Regorafenib |
| P21802 | Fibroblast growth factor receptor 2 | Thalidomide, Palifermin, Ponatinib, Regorafenib |
| P22607 | Fibroblast growth factor receptor 3 | Pazopanib, Palifermin, Ponatinib |
| P22455 | Fibroblast growth factor receptor 4 | Palifermin, Ponatinib |
| P02679 | Fibrinogen γ chain dimer | Sucralfate |
| P09769 | Proto-oncogene tyrosine-protein kinase | |
| | FGR | |
| Q9NZU1 | Fibronectin leucine rich transmembrane | |
| | 1 | |
| P36888 | Receptor-type tyrosine-protein kinase | Sorafenib, Sunitinib, Ponatinib |
| | FLT3 | |
| P49771 | Fms-related tyrosine kinase 3 ligand | |
| P35916 | Vascular endothelial growth factor | Pazopanib, Axitinib, Sunitinib, Sorafenib, |
| | receptor 3 | Regorafenib |
| P02751 | Fibronectin-1 Fragment 3 | Ocriplasmin |
| P02751 | Fibronectin-1 Fragment 4 | Ocriplasmin |
| P02751 | Fibronectin | Ocriplasmin |
| Q04609 | Prostate-specific membrane antigen | Capromab |
| Q92765 | Frizzled-related protein 3, secreted | |
| P19883 | Follistatin | |
| O95633 | Follistatin-like 3 | |
| P02794 | Ferritin | |
| P02792 | | |
| P21217 | Fucosyltransferase 3 | |
| Q11128 | Fucosyltransferase 5 | |
| P06241 | Proto-oncogene tyrosine-protein kinase | Dasatinib |
| | Fyn | |
| P06241 | Proto-oncogene tyrosine-protein kinase | Dasatinib |
| | Fyn | |
| P04406 | Glyceraldehyde-3-phosphate | |
| | dehydrogenase | |
| P54826 | Growth Arrest Specific 1 | |
| P01275 | Glucagon | |
| Q14397 | Glucokinase (hexokinase 4) | |
| | regulator/GCKR | |
| O95390 | Growth-differentiation factor 11 | |
| Q9UK05 | Growth-differentiation factor 2 | |
| P43026 | Bone morphogenetic protein-14 | |
| O60383 | Growth-differentiation factor 9 | |
| P50395 | Rab GDP dissociation inhibitor β | |
| P14136 | Glial fibrillary acidic protein | |
| P56159 | GDNF family receptor α-1 | |
| O00451 | GDNF family receptor α-2 | |
| O60609 | GDNF family receptor α-3 | |
| P10912 | Growth hormone receptor | Somatropin recombinant, Pegvisomant |
| P22749 | Granulysin | |
| P15586 | N-acetylglucosamine-6-sulfatase | |
| P17174 | Aspartate aminotransferase | L-Cysteine, L-Aspartic Acid |
| P07359 | Platelet Glycoprotein Ib α | |
| Q9HCN6 | GPVI/Platelet Glycoprotein VI | |
| Q8N158 | Glypican-2 | |
| P51654 | Glypican 3 | |
| P78333 | Glypican-5 | |
| P06744 | Glucose phosphate isomerase | |
| Q14956 | Osteoactivin/GPNMB | |
| Q8IZF4 | G-protein coupled receptor 114 | |
| Q96D09 | G protein-coupled receptor associated | |
| | sorting protein 2 | |
| P24298 | Alanine aminotransaminase 1 | L-Alanine, Phenelzine |
| O75791 | GRAP2/GRB2-related adaptor protein 2 | |
| O60565 | Gremlin-1 | |
| P28799 | Progranulin | |
| P49840 | Glycogen synthase kinase-3 α/β | |
| P49841 | | |
| P49840 | Glycogen synthase kinase-3 α/β | |
| P49841 | | |
| P06396 | Gelsolin | |
| Q16772 | Glutathione S-transferase A3 | Glutathione |
| P09211 | Glutathione S-transferase Pi 1 | Glutathione, Clomipramine |
| P12544 | Granzyme A | |
| P10144 | Granzyme B | |
| P20718 | Granzyme H | |
| P0C0S5 | Histone H2A.z | |
| P81172 | LEAP-1/Hepcidin | |
| P10915 | Hyaluronan and proteoglycan link | |
| | protein 1 | |
| O14929 | Histone acetyltransferase 1 | |
| Q8TDQ0 | Hepatitis A virus cellular receptor | |
| | 2/Tim-3 | |
| P69905, | Hemoglobin | |
| P68871 | | |
| Q99075 | Heparin-binding EGF-like growth factor | |
| P08631 | Hemopoietic cell kinase | Bosutinib |
| Q9BY41 | Histone deacetylase 8 | Vorinostat |
| Q7Z4V5 | Hepatoma-derived growth factor- | |
| | related protein 2 | |
| Q6ZVN8 | Hemojuvelin | |
| P14210 | Hepatocyte growth factor | |
| Q04756 | Hepatocyte growth factor activator | |
| P31937 | 3-hydroxyisobutyrate dehydrogenase | |
| P49773 | Histidine triad nucleotide binding | Adenosine monophosphate |
| | protein 1 | |
| Q9H422 | Homeodomain-interacting protein | |
| | kinase 3 | |
| P16403 | Histone H1.2 | |
| P09429 | High-mobility group box 1/amphoterin | |
| P04035 | HMG-CoA reductase | Atorvastatin, Fluvastatin, Pravastatin, Pitavastatin, |
| | | Lovastatin, Rosuvastatin, Simvastatin |
| P30519 | Heme oxygenase 2 | |
| P22626 | Heterogeneous nuclear | |
| | ribonucleoprotein A2/B1 | |
| Q99729 | Heterogeneous nuclear | |
| | ribonucleoprotein AB | |
| P61978 | Heterogeneous nuclear | |
| | ribonucleoprotein K | |
| P00738 | Haptoglobin | |
| P15428 | 15-hydroxyprostaglandin | |
| | dehydrogenase [NAD+] | |
| P02790 | Hemopexin | |
| P04196 | Histidine-proline-rich glycoprotein | |
| O60243 | Heparan-sulfate 6-O-sulfotransferase 1 | |
| P14061 | Estradiol 17-β-dehydrogenase 1 | Equilin |
| Q99714 | 3-hydroxyacyl-CoA dehydrogenase type- | |
| | 2 | |
| P07900 | HSP 90α/β | |
| P08238 | | |
| P07900 | HSP 90α/β | |
| P08238 | | |
| P08107 | Hsp70 | |
| P11142 | Heat shock cognate 71 kDa protein | |
| P10809 | Hsp60 | |
| O43464 | High temperature requirement serine | |
| | peptidase A2 | |
| P21815 | Bone sialoprotein 2 | |
| P05362 | Intercellular adhesion molecule 1 | Hyaluronan, Natalizumab |
| P13598 | Intercellular adhesion molecule 2 | |
| P32942 | Intercellular adhesion molecule 3 | |
| Q9UMF0 | Intercellular adhesion molecule 5 | |
| Q9Y6W8 | Inducible T-cell co-stimulator | |
| O75144 | B7 homolog 2/ICOS ligand | |
| P14735 | Insulin-degrading enzyme | Insulin, Bacitracin, Insulin Regular |
| P22304 | Iduronate 2-sulfatase | |
| P35475 | α-L-iduronidase | |
| P01563 | Inferferon-α2 | |
| P01579 | Inferferon-γ | Glucosamine, Olsalazine |
| P15260 | Inferferon-γ Receptor 1 | Interferon gamma-1b |
| P05019 | Insulin-like growth factor I | |
| P08069 | Insulin-like growth factor I receptor | Insulin, Insulin Glargine, Insulin Regular, Insulin |
| | | Lispro, Mecasermin |
| P11717 | Insulin-like growth factor II receptor | Mecasermin |
| P08833 | Insulin-like growth factor-binding | |
| | protein 1 | |
| P18065 | Insulin-like growth factor-binding | |
| | protein 2 | |
| P17936 | Insulin-like growth factor-binding | Mecasermin |
| | protein 3 | |
| P22692 | Insulin-like growth factor-binding | |
| | protein 4 | |
| P24593 | Insulin-like growth factor-binding | |
| | protein 5 | |
| P24592 | Insulin-like growth factor-binding | |
| | protein 6 | |
| Q16270 | Insulin-like growth factor-binding | Insulin, Insulin Regular |
| | protein 7 | |
| P01880 | Immunoglobulin D | |
| P01854 | Immunoglobulin E | |
| P01857 | Immunoglobulin G | |
| P01857 | Immunoglobulin G | |
| P01871 | Immunoglobulin M | |
| P22301 | Interleukin-10 | |
| Q08334 | Interleukin-10 receptor β | |
| P20809 | Interleukin-11 | |
| Q14626 | Interleukin-11 receptor α | Oprelvekin |
| P29459, | Interleukin-12 | |
| P29460 | | |
| P29460, | Interleukin-23 | |
| Q9NPF7 | | |
| P42701 | Interleukin-12 receptor β1 | |
| Q99665 | Interleukin-12 receptor β2 | |
| P35225 | Interleukin-13 | |
| P78552 | Interleukin-13 receptor α1 | |
| Q13261 | Interleukin-15 receptor α | |
| Q14005 | Interleukin-16 | |
| Q16552 | Interleukin-17 | |
| Q9UHF5 | Interleukin-17B | |
| Q8TAD2 | Interleukin-17D | |
| Q96PD4 | Interleukin-17F | |
| Q96F46 | Interleukin-17 receptor A | |
| Q9NRM6 | interleukin-17 receptor B | |
| Q8NAC3 | Interleukin-17 receptor C | |
| Q8NFM7 | Interleukin-17 receptor D | |
| O95998 | Interleukin-18 binding protein | |
| Q13478 | Interleukin-18 receptor 1 | |
| O95256 | Interleukin-18 receptor accessory | |
| | protein | |
| Q9UHD0 | Interleukin-19 | |
| P01583 | Interleukin-1α | Rilonacept |
| P01584 | Interleukin-1β | Rilonacept, Gallium nitrate, Canakinumab, Minocycline |
| Q9NZH6 | Interleukin-37 | |
| P14778 | Interleukin-1 receptor 1 | Anakinra |
| Q9NPH3 | Interleukin-1 Receptor accessory | |
| | protein | |
| Q9NP60 | Interleukin-1 receptor accessory | |
| | protein-like 2/IL-1 sR9 | |
| Q01638 | Interleukin-1 receptor 4 | |
| Q9HB29 | Interleukin-1 receptor-like 2 | |
| P60568 | Interleukin-2 | |
| Q9NYY1 | Interleukin-20 | |
| Q9UHF4 | Interleukin-20 receptor subunit α | |
| Q9GZX6 | Interleukin-22 | |
| Q8N6P7 | Interleukin-22 receptor α-1 | |
| Q969J5 | Interleukin-22 receptor subunit α-2 | |
| Q5VWK5 | Interleukin-23 receptor | |
| Q13007 | Interleukin-24 | |
| Q9H293 | Interleukin-17E | |
| Q8NEV9 | Interleukin-27 | |
| Q6UWB1 | Interleukin-27 receptor subunit α | |
| Q8IZJ0 | Inferferon-λ2 | |
| Q8IU54 | Inferferon-λ1 | |
| P01589 | Interleukin-2 receptor α chain | Denileukin diftitox, Daclizumab, Basiliximab, Aldesleukin |
| P31785 | Interleukin-2 receptor γ chain | Denileukin diftitox, Aldesleukin |
| P08700 | Interleukin-3 | Amlexanox |
| Q6ZMJ4 | Interleukin-34 | |
| P26951 | Interleukin-3 receptor α | Sargramostim |
| P05112 | Interleukin-4 | |
| P24394 | Interleukin-4 receptor α chain | |
| P05113 | Interleukin-5 | Pranlukast |
| Q01344 | Interleukin-5 receptor α | |
| P05231 | Interleukin-6 | Ginseng |
| P08887 | Interleukin-6 receptor α chain | Tocilizumab |
| P40189 | Interleukin-6 receptor subunit β/gp130 | |
| P13232 | Interleukin-7 | |
| P16871 | Interleukin-7 receptor subunit α | |
| P10145 | Interleukin-8 | |
| P20839 | IMP (inosine 5′-monophosphate) | Mycophenolic acid, Ribavirin, Mycophenolate mofetil |
| | dehydrogenase 1 | |
| P12268 | IMP (inosine 5′-monophosphate) | Mycophenolic acid, Mycophenolate mofetil |
| | dehydrogenase 2 | |
| Q9UK53 | Inhibitor of growth 1 | |
| P08476 | Activin A/Inhibin β-A homodimer | |
| P08476 | Activin AB/Inhibin β-A: β-B heterodimer | |
| P09529 | | |
| P01308 | Insulin | |
| P06213 | Insulin receptor | Insulin, Insulin Glulisine, Insulin Aspart, Insulin, |
| | | Insulin Detemir, Insulin Glargine, Insulin Regular, Insulin |
| | | Lispro, Mecasermin |
| P56199, | Integrin α-1: β-1 complex | |
| P05556 | | |
| P08514 | Integrin α-IIb: β-3 complex | |
| P05106 | | |
| P06756, | Integrin α-V: β-5 complex | |
| P18084 | | |
| Q14624 | Inter-α-trypsin inhibitor heavy chain H4 | |
| P78504 | Jagged-1 | |
| Q9Y219 | Jagged-2 | |
| O60674 | Janus kinase 2 | Tofacitinib, Ruxolitinib |
| P57087 | Junctional adhesion molecule B | |
| Q9BX67 | Junctional adhesion molecule C | |
| Q92794 | Histone acetyltransferases monocytic | |
| | leukemic zinc-finger protein | |
| P35968 | Vascular endothelial growth factor | Sunitinib, Sorafenib, Regorafenib, Pazopanib, |
| | receptor 2 | Axitinib, Cabozantinib, Ponatinib |
| Q02241 | Kinesin family member 23 | |
| Q99706 | Killer cell immunoglobulin-like receptor | |
| | 2DL4 | |
| P43630 | Killer cell immunoglobulin-like receptor | |
| | 3DL2 | |
| Q14943 | Killer cell immunoglobulin-like receptor | |
| | 3DS1 | |
| Q8IZU9 | Kirrel3 | |
| P10721 | Stem cell factor receptor/CD117/c-Kit | Pazopanib, Dasatinib, Sunitinib, Sorafenib, |
| | | Nilotinib, Ponatinib, Imatinib, Regorafenib |
| Q9UBX7 | Kallikrein 11 | |
| Q9UKR0 | Kallikrein 12 | |
| Q9UKR3 | Kallikrein-13 | |
| Q9P0G3 | Kallikrein 14 | |
| P07288 | PSA | |
| P07288, | PSA: α-1-antichymotrypsin complex | |
| P01011 | | |
| Q9Y5K2 | Kallikrein 4 | |
| Q9Y337 | Kallikrein 5 | |
| Q92876 | Kallikrein 6 | |
| P49862 | Kallikrein 7 | |
| O60259 | Kallikrein 8 | |
| P03952 | Prekallikrein | |
| Q9NZS2 | Killer cell lectin-like receptor subfamily | |
| | F, member 1 | |
| P26718 | Natural killer group 2 member D | |
| P01042 | Kininogen-1, HMW, Single chain | |
| P01042 | Kininogen-1, HMW, Single chain | |
| P52292 | Karyopherin α 2 (RAG cohort 1, importin | |
| | α-1) | |
| Q14974 | Importin β1 | |
| P01116 | KRAS | |
| Q8NCW0 | Kremen protein 2 | |
| P05783 | Keratin 18 | |
| Q16719 | Kynureninase | L-Alanine |
| P32004 | Neural cell adhesion molecule L1 | (None found) |
| P18627 | Lymphocyte-activation gene 3/LAG-3 | |
| P25391, | Laminin | |
| P07942, | | |
| P11047 | | |
| Q6UX15 | Layilin | |
| P18428 | Lipopolysaccharide-binding protein | |
| P06239 | Proto-oncogene tyrosine-protein kinase | Dasatinib, Ponatinib |
| | LCK | |
| P06239 | Proto-oncogene tyrosine-protein kinase | Dasatinib, Ponatinib |
| | LCK | |
| Q9UIC8 | Leucine carboxyl methyltransferase 1 | L-Leucine |
| P80188 | Lipocalin 2 | |
| Q8N3X6 | Transcription factor MLR1 | |
| P07195 | Lactate dehydrogenase 1 (heart) | |
| P41159 | Leptin | |
| P48357 | Leptin receptor | |
| P05162 | Galectin-2 | |
| P17931 | Galectin-3 | |
| Q08380 | Galectin-3 binding protein | |
| P56470 | Galectin-4 | |
| O00214 | Galectin-8 | |
| Q99538 | Legumain | |
| P42702 | Leukemia inhibitory factor receptor | |
| | extracellular domain | |
| Q8NHL6 | Leukocyte immunoglobulin-like receptor | |
| | subfamily B member 1 | |
| Q8N423 | Leukocyte immunoglobulin-like receptor | |
| | subfamily B member 2 | |
| Q9HAP6 | Protein lin-7 homolog B | |
| P20700 | Lamin-B1 | |
| P22079 | Lactoperoxidase | |
| Q6UXM1 | Leucine-rich repeats and Ig-like domains | |
| | protein 3 | |
| Q14114 | Apolipoprotein E receptor 2/LRP8 | |
| P30533 | α-2-macroglobulin receptor-associated | |
| | protein | |
| Q86UE6 | Leucine-rich repeat transmembrane | |
| | neuronal protein 1 | |
| Q86VH5 | Leucine-rich repeat transmembrane | |
| | neuronal protein 3 | |
| Q13449 | Limbic system-associated membrane | |
| | protein | |
| P01374 | Tumor necrosis factor ligand | Etanercept |
| | superfamily member 1/TNF- | |
| | β/Lymphotoxin-α | |
| P01374, | Lymphotoxin α1: β2 | |
| Q06643 | | |
| P01374, | Lymphotoxin α2: β1 | |
| Q06643 | | |
| P09960 | Leukotriene A-4 hydrolase | |
| P36941 | Lymphotoxin β receptor | |
| P02788 | Lactoferrin | |
| O95711 | Lymphocyte antigen 86/Myeloid | |
| | differentiation 1 | |
| Q9HBG7 | T-lymphocyte surface antigen Ly- | |
| | 9/CD229 | |
| P07948 | Lyn kinase, isoform B | Bosutinib, Ponatinib |
| P07948 | Lyn kinase | Bosutinib, Ponatinib |
| Q9Y5Y7 | Lymphatic vessel endothelial hyaluronic | |
| | acid receptor 1 | |
| P61626 | Lysozyme | L-Aspartic Acid |
| Q02750 | MAPK kinase 1 | Bosutinib, Trametinib |
| P36507 | MAPK kinase 2 | Bosutinib, Trametinib |
| P45985 | MAPK kinase 4 | |
| O43318 | TAK1-TAB1 fusion | |
| Q15750 | | |
| P28482 | MAPK 1 | Isoprenaline, Arsenic trioxide |
| Q15759 | MAPK 11 | Regorafenib |
| P53778 | MAPK 12 | |
| O15264 | MAPK 13 | |
| Q16539 | MAPK 14 | |
| P27361 | MAPK 3/ERK-1 | Arsenic trioxide, Sulindac |
| P45983 | MAPK 8 | |
| P45984 | Mitogen-activated protein kinase | |
| | 9/JNK2 | |
| P49137 | MAPK-activated protein kinase 2 | |
| Q16644 | MAPK-activated protein kinase 3 | |
| Q8IW41 | MAPK-activated protein kinase 5 | |
| P10636 | Microtubule-associated protein tau | Paclitaxel, Docetaxel |
| P48740 | Mannan-binding lectin serine peptidase | |
| | 1 | |
| P42679 | Megakaryocyte-associated tyrosine- | |
| | protein kinase | |
| O00339 | Matrilin-2 | |
| O15232 | Matrilin-3 | |
| P02144 | Myoglobin | |
| O95243 | Methyl-CpG-binding domain protein 4 | |
| P11226 | Mannose-binding protein C | |
| P40925 | Malate dehydrogenase, cytoplasmic | |
| P21741 | Midkine | |
| Q00987 | MDM2 ubiquitin ligase | |
| Q15648 | Mediator complex subunit 1 | |
| Q9NQ76 | Matrix extracellular | |
| | phosphoglycoprotein | |
| P08581 | Hepatocyte growth factor receptor/c- | Cabozantinib |
| | Met | |
| P53582 | Methionine aminopeptidase 1 | Nitroxoline |
| P50579 | Methionine aminopeptidase 2 | L-Methionine |
| Q08431 | Milk fat globule-EGF factor 8 | |
| Q9BY79 | Membrane frizzled-related | |
| | protein/MFRP | |
| Q16674 | Melanoma Inhibitory Activity | |
| Q29983 | MHC class I chain-related protein A | |
| Q29980 | MICB/MHC class I polypeptide-related | |
| | sequence B | |
| P14174 | MIF/macrophage migration inhibitory | |
| | factor | |
| Q495T6 | Neprilysin-2 | |
| P03956 | Matrix metalloproteinase 1/ | Marimastat |
| | collagenase 1 | |
| P09238 | Matrix metalloproteinase | Marimastat |
| | 10/Stromelysin 2 | |
| P39900 | Matrix metalloproteinase | Acetohydroxamic Acid, Marimastat |
| | 12/Macrophage metalloelastase | |
| P45452 | Matrix metalloproteinase | Marimastat |
| | 13/Collagenase 3 | |
| P50281 | Matrix metalloproteinase | Marimastat |
| | 14/Membrane type matrix | |
| | metalloproteinase 1 | |
| P51512 | Matrix metalloproteinase | Marimastat |
| | 16/Membrane-type matrix | |
| | metalloproteinase 3 | |
| | Matrix metalloproteinase | |
| Q9ULZ9 | 17/Membrane-type matrix | Marimastat |
| | metalloproteinase 4 | |
| P08253 | Matrix metalloproteinase | Captopril, Marimastat |
| | 2/Gelatinase A | |
| P08254 | Matrix metalloproteinase | Marimastat |
| | 3/Stromelysin 1 | |
| P09237 | Matrix metalloproteinase | Marimastat |
| | 7/Matrilysin | |
| P22894 | Matrix metalloproteinase | Marimastat |
| | 8/Neutrophil collagenase | |
| P14780 | Matrix metalloproteinase | Captopril, Glucosamine, Minocycline, Marimastat |
| | 9/Gelatinase B | |
| P40238 | Thrombopoietin Receptor | Eltrombopag, Romiplostim |
| P05164 | Myeloperoxidase | Mesalazine, Melatonin, L-Carnitine, Cefdinir |
| P22897 | Macrophage mannose receptor | |
| Q9UBG0 | Macrophage mannose receptor 2 | |
| Q13421 | Mesothelin | |
| Q13421 | Mesothelin | |
| P26038 | Moesin | |
| P21757 | Macrophage scavenger receptor | |
| P26927 | Macrophage stimulatory protein | |
| Q04912 | Macrophage stimulatory protein | |
| | receptor | |
| Q02083 | Acid ceramidase-like protein | |
| Q13765 | Nascent polypeptide-associated | |
| | complex α subunit | |
| Q9UJ70 | N-acetyl-D-glucosamine kinase | N-Acetyl-D-glucosamine |
| P43490 | Visfatin | |
| Q9H9S0 | Homeobox transcription factor Nanog | |
| P54920 | N-ethylmaleimide-sensitive factor | |
| | attachment protein α | |
| P41271 | Neuroblastoma suppressor of | |
| | tumorigenicity 1 | |
| P13591 | Neural cell adhesion molecule 1, 120 | |
| | kDa isoform | |
| P16333 | NCK adaptor protein 1 | |
| O76036 | NKp46/NCR1/natural cytotoxicity | |
| | triggering receptor 1 | |
| O95944 | Natural cytotoxicity triggering | |
| | receptor 2 | |
| O14931 | Natural cytotoxicity triggering | |
| | receptor 3 | |
| P01138 | β-nerve growth factor | Clenbuterol |
| P14543 | Nidogen | Urokinase |
| Q14112 | Nidogen-2 | |
| Q8N0W4 | Neuroligin 4, X-linked | |
| P15531 | Nucleoside diphosphate kinase A | |
| P22392 | Nucleoside diphosphate kinase B | |
| P30419 | N-myristoyltransferase 1 | |
| Q13253 | Noggin | |
| Family | Protein kinase B (RAC family) | |
| Family | Protein kinase B (RAC family) | |
| Non-human | APOA1_MOUSE | |
| 0 | 0 | |
| P46531 | Notch 1 | |
| Q04721 | Notch 2 | |
| Q9UM47 | Notch 3 | |
| P48745 | Nephroblastoma Overexpressed gene | Insulin, Insulin Regular |
| | homolog | |
| P01161 | 0 | |
| P16860 | Brain natriuretic peptide 32 | Carvedilol |
| P20393 | NR1D1/nuclear receptor subfamily 1, | |
| | group D, member 1 | |
| P04150 | Glucocorticoid receptor | Halobetasol Propionate, Megestrol acetate, |
| | | Budesonide, Difluprednate, Clobetasol propionate, |
| | | Flunisolide, Flumethasone Pivalate, Prednisone, |
| | | Diflorasone, Betamethasone, Desonide, Fluocinolone |
| | | Acetonide, Clocortolone, Mifepristone, Amcinonide, |
| | | Paramethasone, Fluticasone furoate, Cortisone |
| | | acetate, Fluocinonide, Methylprednisolone, Fluticasone |
| | | Propionate, Flurandrenolide, Fluoxymesterone, |
| | | Alclometasone, Hydrocortamate, Loteprednol, |
| | | Beclomethasone, Hydrocortisone, Prednicarbate, |
| | | Prednisolone, Ciclesonide, Desoximetasone, Medrysone, |
| | | Triamcinolone, Fludrocortisone, Fluorometholone, |
| | | Rimexolone, Mometasone, Dexamethasone |
| Q92823 | NRCAM/neuronal cell adhesion | |
| | molecule | |
| Q02297 | Neuregulin-1 | |
| O14786 | Neuropilin-1 | Palifermin, Pegaptanib |
| P58400 | Neurexin-1-β | |
| Q9HDB5 | Neurexin-3-β | |
| Q9UNZ2 | NSFL1 cofactor p47 | |
| P20783 | Neurotrophin-3 | |
| P34130 | Neurotrophin-5 | |
| Q9HB63 | Netrin-4 | |
| P04629 | Neurotrophic tyrosine kinase receptor | Amitriptyline, Imatinib, Regorafenib |
| | type 1 | |
| Q16620 | Neurotrophic tyrosine kinase receptor | Amitriptyline |
| | type 2 | |
| Q16288 | Neurotrophic tyrosine kinase receptor | |
| | type 3 | |
| Q8IVD9 | NudC domain-containing protein 3 | |
| P58417 | Neurexophilin-1 | |
| Q9NX40 | Ovarian cancer immunoreactive antigen | |
| | domain containing 1 | |
| Q6UX06 | Olfactomedin-4 | |
| P78380 | Oxidized low-density lipoprotein | |
| | receptor 1 | |
| Q99983 | Osteomodulin/Osteoadherin | |
| Q14982 | Opioid-binding cell adhesion molecule | |
| P13725 | Oncostatin M | |
| P07237 | Protein disulfide-isomerase | |
| Q9UQ80 | ErbB3 binding protein Ebp1 | |
| P68402 | Platelet-activating factor | |
| | acetylhydrolase IB subunit | |
| | β/PAFAH subunit β | |
| O75914 | p21-activated kinase 3 | |
| Q9NQU5 | p21-activated kinase 6 | |
| Q9P286 | p21-activated kinase 7 | |
| Q13219 | Pregnancy-associated plasma protein-A | |
| Q99497 | PARK7/Parkinson protein 7 | |
| P12004 | Proliferating cell nuclear antigen | |
| Q16549 | Proprotein Convertase 7 | |
| Q9BQ51 | Programmed cell death 1 ligand 2 | |
| Q9HCR9 | cAMP and cGMP phosphodiesterase | Tadalafil |
| | 11A/PDE11A | |
| O00408 | Phosphodiesterase 2A, cGMP- | Tofisopam |
| | stimulated | |
| Q14432 | cGMP-inhibited cAMP | Levosimendan, Cilostazol, Anagrelide, Tofisopam, |
| | phosphodiesterase 3A/PDE3A | Amrinone, Oxtriphylline, Ibudilast, Milrinone, |
| | | Aminophylline, Enoximone, Theophylline |
| Q08499 | cAMP-specific phosphodiesterase | Dyphylline, Roflumilast, Adenosine |
| | 4D/PDE4D | monophosphate, Iloprost, Ibudilast, Ketotifen |
| O76074 | cGMP-binding cGMP-specific | Dipyridamole, Udenafil, Avanafil, Vardenafil, Sildenafil, |
| | phosphodiesterase/PDE5A | Tadalafil, Pentoxifylline, Theophylline |
| Q13946 | High affinity cAMP-specific | Dyphylline, Ketotifen |
| | phosphodiesterase 7A/PDE7A | |
| O76083 | High affinity cAMP-specific | |
| | phosphodiesterase 9A/PDE9A | |
| P04085 | Platelet-derived growth factor A chain | |
| | homodimer | |
| P01127 | Platelet-derived growth factor B chain | |
| | homodimer | |
| Q9NRA1 | Platelet-derived growth factor C chain | |
| | homodimer | |
| P09619 | Platelet-derived growth factor receptor | Pazopanib, Dasatinib, Becaplermin, Sunitinib, Sorafenib, |
| | β-type | Imatinib, Regorafenib |
| P30101 | Protein disulfide isomerase A3 | |
| Q15118 | Pyruvate dehydrogenase kinase, | |
| | isozyme 1 | |
| O15530 | 3-phosphoinositide-dependent protein | Celecoxib |
| | kinase 1 | |
| Q96GD0 | Pyridoxal phosphate phosphatase | |
| P30086 | Phosphatidylethanolamine-binding | |
| | protein 1 | |
| P16284 | Platelet endothelial cell adhesion | |
| | molecule | |
| O00541 | Pescadillo | |
| P02776 | Platelet factor 4 | Drotrecogin alfa |
| Q99471 | Prefoldin subunit 5 | |
| P18669 | Phosphoglycerate mutase 1 | |
| P52209 | 6-Phosphogluconate dehydrogenase | Ketotifen, Dacarbazine, Gadopentetate dimeglumine |
| P49763 | Placenta growth factor | Aflibercept |
| P00558 | Phosphoglycerate kinase 1 | |
| O75594 | Peptidoglycan recognition protein, short | |
| P19957 | Elafin | |
| P01833 | Polymeric immunoglobulin receptor | |
| P42336 | Phosphoinositide-3-kinase catalytic α | |
| P27986 | polypeptide: regulatory subunit 1α | |
| | complex | |
| P48736 | Phosphatidylinositol-4,5-bisphosphate | |
| | 3-kinase catalytic subunit γ isoform | |
| P11309 | Proto-oncogene serine/threonine- | Adenosine monophosphate |
| | protein kinase Pim-1 | |
| P14618 | M2-pyruvate kinase | Pyruvic acid |
| O15496 | Phospholipase A2, Group X | |
| P04054 | Phospholipase A2, Group IB | Niflumic Acid |
| P14555 | Phospholipase A2, Group IIA | Indomethacin, Diclofenac, Suramin, Ginkgo biloba |
| Q9NZK7 | Phospholipase A2, Group IIE | Aminosalicylic Acid |
| P39877 | Phospholipase A2, Group V | |
| Q13093 | Platelet-activating factor | |
| | acetylhydrolase/LDL-associated | |
| | phospholipase A2 | |
| P00750 | Tissue-type plasminogen activator | Aminocaproic Acid, Iloprost, Urokinase, Ibuprofen |
| P00749 | Urokinase-type plasminogen activator | Urokinase, Amiloride |
| Q03405 | Urokinase plasminogen activator | Anistreplase, Urokinase, Tenecteplase, Reteplase, |
| | surface receptor | Alteplase |
| P19174 | Phospholipase C-II | |
| P00747 | Angiostatin | Streptokinase, Anistreplase, Aminocaproic Acid, |
| | | Urokinase, Reteplase, Alteplase, Aprotinin, Tranexamic |
| | | Acid, Tenecteplase |
| P00747 | Plasmin | Streptokinase, Anistreplase, Aminocaproic Acid, |
| | | Urokinase, Reteplase, Alteplase, Aprotinin, Tranexamic |
| | | Acid, Tenecteplase |
| P00747 | Plasminogen | Streptokinase, Anistreplase, Aminocaproic Acid, |
| | | Urokinase, Reteplase, Alteplase, Aprotinin, Tranexamic |
| | | Acid, Tenecteplase |
| P53350 | Serine-threonine-protein kinase PLK1 | |
| O60486 | Plexin C1 | |
| P01189 | β-Endorphin | Loperamide |
| P01189 | Adrenocorticotropic hormone | Loperamide |
| P27169 | Paraoxonase 1 | Cefazolin |
| P16435 | NADPH-P450 Oxidoreductase | Flavin adenine dinucleotide |
| Q13950 | Osteoblast-specific transcription | |
| | factor 2 | |
| Q15181 | Inorganic pyrophosphatase | |
| P02775 | Neutrophil-activating peptide 2 | |
| P02775 | Neutrophil-activating peptide 2 | |
| P02775 | Connective-tissue activating peptide III | |
| P62937 | Peptidylprolyl isomerase A | Cyclosporine, L-Proline |
| | (Cyclophilin A) | |
| P62937 | Peptidylprolyl isomerase A | Cyclosporine, L-Proline |
| | (Cyclophilin A) | |
| P23284 | Cyclophilin B | L-Proline |
| Q08752 | Peptidylprolyl isomerase D | |
| Q9UNP9 | Peptidylprolyl isomerase E | |
| | (Cyclophilin E) | |
| P30405 | Peptidylprolyl isomerase F | L-Proline |
| | (Cyclophilin F) | |
| Q08209 | Calcineurin | |
| P63098 | | |
| P63098 | Calcineurin subunit B type 1 | |
| P01298 | Pancreatic hormone | |
| Q06830 | Peroxiredoxin-1 | |
| P30044 | Peroxiredoxin-5 | Auranofin |
| P30041 | Peroxiredoxin-6 | |
| Q13131 | AMP Kinase (α1β1γ1) | |
| Q9Y478 | | |
| P54619 | | |
| P54646 | | |
| O43741 | AMP Kinase (α2β2γ2) | |
| P54619 | | |
| P17612 | cAMP-dependent protein kinase | |
| | catalytic subunit α | |
| P17252 | Protein kinase C α | Phosphatidylserine, Ingenol Mebutate, Vitamin E |
| P05771 | Protein kinase C β type (splice variant II) | Vitamin E |
| Q05655 | Protein kinase C λ | Ingenol Mebutate |
| P05129 | Protein kinase C γ | |
| P41743 | Protein kinase C ι | |
| Q04759 | Protein kinase C θ | |
| Q05513 | Protein kinase C ζ | |
| P01236 | Prolactin | |
| P16471 | Prolactin receptor | Somatropin recombinant, Fluoxymesterone |
| P04070 | Protein C | Menadione, Sodium Tetradecyl Sulfate |
| P04070 | Activated Protein C | Menadione, Sodium Tetradecyl Sulfate |
| P04070 | Activated Protein C | Menadione, Sodium Tetradecyl Sulfate |
| P58294 | Endocrine-gland-derived vascular | |
| | endothelial growth factor | |
| P07225 | Protein S | Menadione, Sodium Tetradecyl Sulfate, Drotrecogin alfa |
| P07477 | Trypsin | Aprotinin |
| P07478 | Trypsin-2 | |
| Q9GZN4 | Brain-specific serine protease 4 | |
| Q9BQR3 | Marapsin | |
| P35030 | Trypsin-3 | |
| P98073 | Enterokinase | |
| P24158 | Proteinase-3 | |
| P25786 | Proteasome subunit α1 | |
| P25787 | Proteasome subunit α2 | |
| P60900 | Proteasome subunit α type 6 | |
| P51665 | Proteasome subunit p40 | |
| Q06323 | Proteasome activator subunit 1 | |
| P61289 | Proteasome activator complex subunit 3 | |
| O60542 | Persephin | |
| P60484 | Phosphatase and tensin homolog | |
| P35354 | Cyclooxygenase-2 | Indomethacin, Niflumic Acid, Dihomo-γ_-linolenic |
| | | acid, Sulfasalazine, Nepafenac, Etoricoxib, Lumiracoxib, |
| | | Bromfenac, Antipyrine, Tenoxicam, Fenoprofen, Lornoxicam, |
| | | Naproxen, Etodolac, Oxaprozin, Ginseng, Piroxicam, |
| | | Trisalicylate-choline, Thalidomide, Flurbiprofen, |
| | | Salicylate-sodium, Acetylsalicylic acid, Mefenamic |
| | | acid, Meloxicam, Diflunisal, Meclofenamic acid, |
| | | Mesalazine, Sulindac, Acetaminophen, Salsalate, |
| | | Aminosalicylic Acid, Celecoxib, Diclofenac, Ibuprofen, |
| | | Nabumetone, Balsalazide, Pomalidomide, Ketoprofen, |
| | | Tolmetin, Carprofen, Tiaprofenic acid, Phenylbutazone, |
| | | Ketorolac, Salicyclic acid, Antrafenine, Suprofen, |
| | | Magnesium salicylate, Lenalidomide, Icosapent |
| P01270 | Parathyroid hormone | |
| P12272 | Parathyroid hormone-related protein | |
| Q05397 | Focal adhesion kinase 1 | |
| Q13882 | Tyrosine-protein kinase 6 | Vandetanib |
| P21246 | Pleiotrophin | |
| P18031 | Tyrosine-protein phosphatase non- | |
| | receptor type 1 | Tiludronate |
| Q06124 | Tyrosine-protein phosphatase non- | |
| | receptor type 11 | |
| P17706 | Tyrosine-protein phosphatase non- | |
| | receptor type 2 | |
| P29350 | Tyrosine phosphatase SHP-1 | |
| P10082 | Peptide YY | |
| P63000 | Ras-related C3 botulinum toxin | |
| | substrate 1 | |
| Q06609 | DNA repair protein RAD51 homolog 1 | |
| P62826 | GTP-binding nuclear protein Ran | |
| Q99969 | Chemerin | |
| P20936 | RAS p21 protein activator | |
| P06400 | Retinoblastoma 1 | Insulin, Insulin Regular |
| Q14498 | RNA-binding motif protein 39 | |
| P02753 | Retinol-binding protein 4 | |
| Q969Z4 | RELT tumor necrosis factor receptor | |
| P00797 | Renin | Remikiren, Aliskiren |
| P07949 | Proto-oncogene tyrosine-protein kinase | Cabozantinib, Sorafenib, Ponatinib, Regorafenib |
| | receptor Ret | |
| Q9HD89 | Resistin | |
| Q96B86 | Repulsive guidance molecule A | |
| Q6NW40 | RGM domain family member B | |
| Q9HCK4 | Roundabout axon guidance molecule 2, | |
| | ROBO2 | |
| Q96MS0 | Roundabout axon guidance molecule 3, | |
| | ROBO3 | |
| Q01973 | Tyrosine-protein kinase transmembrane | |
| | receptor ROR1 | |
| P62979 | Ubiquitin + 1, truncated mutation for | |
| | UbB | |
| P62979 | Ubiquitin | |
| P23396 | Ribosomal protein S3 | |
| P51812 | Ribosomal protein S6 kinase α-3 | |
| O75582 | Ribosomal protein S6 kinase 5 | |
| P62081 | Ribosomal protein S7 | |
| P08865 | Laminin receptor/ribosomal protein SA | |
| Q6UXX9 | Roof plate-specific spondin-2, isoform 1 | |
| Q9NQC3 | Reticulon-4/Nogo-A | |
| Q9BZR6 | Nogo Receptor/reticulon 4 receptor | |
| P06702 | S100A9/calgranulin B | |
| P02735 | Serum amyloid A | |
| Q9Y3A5 | Ribosome maturation protein SBDS | |
| Q14108 | LIMPII/SCARB2 | |
| Q14162 | Scavenger receptor class F member | |
| | 1/SREC-I | |
| Q96GP6 | Scavenger receptor class F member | |
| | 2/SREC-II | |
| O75556 | Mammaglobin-B | |
| P09683 | Secretin | |
| P16581 | E-Selectin | |
| P14151 | L-Selectin | |
| P16109 | P-Selectin | Dalteparin, Nadroparin, Heparin |
| Q14563 | Semaphorin 3A | |
| O15041 | Semaphorin-3E | |
| Q9H2E6 | Semaphorin-6A | |
| Q9H2E6 | Semaphorin-6A | |
| P01009 | α1-Antitrypsin | |
| P01011 | α1-Antichymotrypsin | |
| P29622 | Kallistatin | |
| P05154 | Protein C Inhibitor | Urokinase, Drotrecogin alfa |
| P08185 | Corticosteroid binding globulin | |
| P05543 | Thyroxine-Binding Globulin | |
| P01008 | Antithrombin III | Tinzaparin, Dalteparin, Nadroparin, Fondaparinux |
| | | sodium, Sulodexide, Ardeparin, Enoxaparin, Heparin |
| P05546 | Heparin cofactor II | Ardeparin, Sulodexide |
| P05121 | Plasminogen activator inhibitor 1 | Anistreplase, Urokinase, Reteplase, Alteplase, |
| | | Tenecteplase, Drotrecogin alfa |
| P07093 | Protease nexin I | |
| P08697 | α2-Antiplasmin | Ocriplasmin |
| P05155 | C1-Esterase Inhibitor | |
| Q01105 | SET nuclear oncogene protein | |
| Q6UXD5 | Seizure 6-like protein 2 | |
| P31947 | 14-3-3σ/Stratifin | |
| Q8N474 | Frizzled-related protein 1, secreted | |
| P35247 | Pulmonary surfactant-associated | |
| | protein D | |
| O43765 | Small glutamine-rich tetratricopeptide | |
| | repeat-containing protein α | |
| O60880 | Signaling lymphocyte activation | |
| | molecule/CDw150 | |
| P04278 | Sex hormone-binding globulin | |
| P29353 | SHC-transforming protein 1 | |
| Q15465 | Sonic Hedgehog | |
| Q9BZZ2 | Sialoadhesin | |
| Q08ET2 | Siglec-14 | |
| O43699 | Siglec-6 | |
| Q9Y286 | Siglec-7 | |
| Q9Y336 | Siglec-9 | |
| Q8IXJ6 | Sirtuin 2 | |
| P63208 | S-phase kinase-associated protein 1 | |
| Q96DU3 | SLAM family member 6/NTB-A | |
| Q9NQ25 | SLAM family member 7/CRACC | |
| Q9H1K4 | Mitochondrial glutamate carrier 2 | |
| O94991 | SLIT and NTRK-like protein 5 | |
| P03973 | Secretory leukocyte protease inhibitor | |
| Q92484 | Sphingomyelin phosphodiesterase, acid- | |
| | like 3A | |
| P62306 | Small nuclear ribonucleoprotein F | |
| O95219 | Sorting nexin 4 | |
| P00441 | Superoxide dismutase [Cu—Zn] | |
| P04179 | Superoxide dismutase [Mn] | |
| Q96PQ0 | Sortilin-related VPS10 domain | |
| | containing receptor 2 | |
| P09486 | Osteonectin | |
| Q14515 | SPARC-like 1 (hevin) | |
| Q9NYA1 | Sphingosine kinase 1 | |
| Q9NRA0 | Sphingosine kinase 2 | |
| O43278 | Hepatocyte growth factor activator | |
| | inhibitor type 1 | |
| O43291 | Kunitz-type protease inhibitor 2A | |
| Q08629 | Testican-1/SPOCK1 | |
| Q92563 | Testican-2/SPOCK2 | |
| Q9HCB6 | Spondin-1 | |
| Q13813 | αII-Spectrin | |
| P12931 | Proto-oncogene tyrosine-protein | Dasatinib, Bosutinib, Ponatinib |
| | kinase Src | |
| Q08945 | FACT complex subunit SSRP1 | |
| P61278 | Somatostatin-28 | Cysteamine |
| Q8WWQ8 | Stabilin-2 | |
| P52823 | Stanniocalcin-1 | |
| P31948 | Stress-induced-phosphoprotein 1 | |
| O75716 | Serine-threonine-protein kinase 16 | |
| O94768 | Serine/threonine kinase 17b | |
| | (STK17B)/DRAK2 | |
| Q16623 | Syntaxin 1A | |
| O60506 | Heterogeneous nuclear | |
| | ribonucleoprotein Q | |
| P09758 | Tumor-associated calcium signal | |
| | transducer 2 | |
| Q9UHD2 | TANK-binding kinase 1 | |
| P20226 | TATA-box-binding protein | |
| P13385 | Cripto-1 | |
| P42680 | Tyrosine-protein kinase Tec | |
| Q02763 | Tyrosine-protein kinase receptor Tie-2, | Vandetanib, Ponatinib, Regorafenib |
| | soluble/Angiopoietin-1 receptor | |
| P02787 | Transferrin | Aluminium |
| Q07654 | Trefoil factor 3 | |
| P10646 | Tissue factor pathway inhibitor | Dalteparin, Coagulation factor VIIa |
| P01266 | Thyroglobulin | |
| P01137 | Transforming growth factor β-1 | Hyaluronidase |
| P61812 | Transforming growth factor β-2 | |
| P10600 | Transforming growth factor β-3 | |
| Q15582 | Transforming growth factor β induced | |
| | protein | |
| P37173 | TGF-β receptor II | |
| Q03167 | Transforming growth factor β receptor | |
| | type III | |
| Q08188 | Transglutaminase 3 | |
| P07996 | Thrombospondin-1 | |
| P35442 | Thrombospondin-2 | |
| P35443 | Thrombospondin-4 | |
| P35590 | Tyrosine-protein kinase receptor Tie-1, | |
| | soluble | |
| P01033 | Tissue inhibitor of metalloproteinases 1 | |
| P16035 | Tissue inhibitor of metalloproteinases 2 | |
| P35625 | Tissue inhibitor of metalloproteinases 3 | |
| P04183 | Thymidine kinase, cytosolic | |
| P29401 | Transketolase | |
| O60603 | Toll-like receptor 2 | OspA lipoprotein |
| O00206 | Toll-like receptor 4 | Naloxone |
| P24821 | Tenascin | |
| P01375 | Tumor necrosis factor ligand | Thalidomide, Chloroquine, golimumab, Adalimumab, |
| | superfamily member 2/TNF-α | Pranlukast, Certolizumab pegol, Clenbuterol, Amrinone, |
| | | Pomalidomide, Glucosamine, Etanercept, Infliximab |
| P98066 | Tumor necrosis factor-inducible gene 6 | |
| | protein | |
| O00220 | Tumor necrosis factor receptor | |
| | superfamily member 10A | |
| Q9UBN6 | Tumor necrosis factor receptor | |
| | superfamily member 10D | |
| Q9Y6Q6 | Receptor activator of NF-KB/RANK | |
| O00300 | Osteoprotegerin/TNFRSF11B | |
| Q9NP84 | TWEAK receptor/TNFRSF12A | |
| O14836 | Tumor necrosis factor receptor | |
| | superfamily member 13B | |
| Q96RJ3 | B-cell-activating factor | |
| | receptor/TNFRSF13C | |
| Q92956 | HVEM/TNFRSF14 | |
| Q02223 | B-cell maturation protein | |
| Q9Y5U5 | GITR/TNFRSF18 | |
| Q9NS68 | TROY/TNFRSF19 | |
| P19438 | Tumor necrosis factor receptor | |
| | superfamily member 1A | |
| P20333 | Tumor necrosis factor receptor | Etanercept |
| | superfamily member 1B | |
| O75509 | Death receptor 6(DR6)/TNFRSF21 | |
| Q93038 | Death receptor 3 (DR3)/TNFRSF25 | |
| P43489 | Tumor necrosis factor receptor | |
| | superfamily member 4 | |
| O95407 | Death decoy receptor 3 | |
| | (DcR3)/TNFRSF6B | |
| P28908 | CD30 | Brentuximab vedotin |
| Q07011 | 4-1BB/CD137 | |
| O14788 | Osteoprotegerin ligand/TRANCE | Denosumab, Lenalidomide |
| O43508 | Tumor necrosis factor ligand | |
| | superfamily member 12 | |
| Q9Y275 | B-cell-activating factor | Belimumab |
| O43557 | LIGHT/TNFSF14 | |
| O95150 | Tumor necrosis factor ligand | |
| | superfamily member 15 | |
| Q9UNG2 | Tumor necrosis factor ligand | |
| | superfamily member 18 | |
| P23510 | OX40 Ligand/Tumor necrosis factor | |
| | ligand superfamily member 4 | |
| P32971 | CD30 Ligand | |
| P41273 | 4-1BB ligand/CD137L | |
| P27768 | 0 | |
| P23693 | 0 | |
| P11387 | Topoisomerase I | Irinotecan, Topotecan, Lucanthone, Sodium |
| | | stibogluconate |
| P60174 | Triosephosphate isomerase | |
| P09493 | Tropomyosin 1 | |
| P07951 | Tropomyosin β chain | |
| P07202 | Thyroid peroxidase | Dextrothyroxine, Propylthiouracil, Carbimazole, |
| | | Methimazole |
| P20231 | Tryptase β-2 | |
| Q9NRR2 | Tryptase γ | |
| P13693 | Fortilin | |
| Q969D9 | Thymic stromal lymphopoietin | |
| O95881 | Thioredoxin domain-containing | Glutathione |
| | protein 12 | |
| P29597 | tyrosine kinase 2 | |
| P04818 | Thymidylate synthase | Pemetrexed, Trimethoprim, Fluorouracil, Leucovorin, |
| | | Gemcitabine, Pralatrexate, Capecitabine, Raltitrexed, |
| | | Trifluridine, Floxuridine |
| Q06418 | Tyrosine-protein kinase receptor TYRO3 | |
| P63279 | SUMO-conjugating enzyme UBC9 | |
| P68036 | Ubiquitin-conjugating enzyme E2 L3 | |
| P61088 | Ubiquitin-conjugating enzyme E2 N | |
| P09936 | Ubiquitin C-terminal hydrolase-L1 | |
| Q9Y3C8 | Ubiquitin-fold modifier-conjugating | |
| | enzyme 1 | |
| P61960 | Ubiquitin-fold modifier 1 | |
| Q9BZM6 | UL16-binding protein 1/NKG2D ligand 1 | |
| Q9BZM5 | UL16-binding protein 2/NKG2D ligand 2 | |
| Q9BZM4 | UL16 binding protein 3 | |
| O95185 | Netrin receptor UNC5H3 | |
| Q6UXZ4 | Netrin receptor UNC5H4 | |
| P19320 | Vascular cell adhesion protein | Carvedilol |
| | 1/VCAM 1 | |
| P15692 | Vascular endothelial growth factor A | Dalteparin, Carvedilol, Gliclazide, Vandetanib, |
| | | Ranibizumab, Bevacizumab, Minocycline, Aflibercept |
| P15692 | Vascular endothelial growth factor A, | Dalteparin, Carvedilol, Gliclazide, Vandetanib, |
| | secreted splice variant | Ranibizumab, Bevacizumab, Minocycline, Aflibercept |
| P49767 | Vascular endothelial growth factor C | |
| P01282 | Vasoactive Intestinal Peptide | |
| Q9NP79 | Dopamine responsive protein | |
| P04275 | von Willebrand factor | Antihemophilic Factor |
| Q8TEU8 | Growth and differentiation factor- | |
| | associated serum protein | |
| | 1/GASP1/WFIKKN2 | |
| Q9Y5W5 | Wnt inhibitory factor 1 | |
| O95388 | WNT1-inducible-signaling pathway | |
| | protein 1 | |
| O00755 | Wingless-type MMTV integration site | |
| | family, member 7A | |
| P47992 | Lymphotactin | |
| Q9NQW7 | X-Pro aminopeptidase 1 | |
| P12956 | ATP-dependent DNA helicase II 70 kDa | |
| | subunit | |
| P07947 | Proto-oncogene tyrosine-protein kinase | Dasatinib |
| | Yes | |
| Family | 14-3-3 protein family | |
| Family | 14-3-3 protein family | |
| P43403 | ZAP70/70 kDa zeta-associated protein | |
| | kinase | |
| P43403 | ZAP70/70 kDa zeta-associated protein | |
| | kinase | |
| P43403 | ZAP70/70 kDa zeta-associated protein | |
| | kinase | |

### Example 2

Table 12 shows proteins that have differential expression in Duchene muscular dystrophy (DMD) and non-DMD subjects identified utilizing the aptamer-based compositions and methods described herein.

TABLE 12
Down in blood in DMD patients (not synthesized in muscle)

| | Gene | | |
|---|---|---|---|
| No | Name | Full name | Average KS |
| 45 | GDF11 | Growth/differentiation factor 11 | −0.80 |
| 51 | RELT | Tumor necrosis factor receptor | −0.78 |
| | | superfamily member 19L | |
| 49 | CD55 | Complement decay-accelerating | −0.74 |
| | | factor | |
| 42 | CADM1 | Nectin-like protein 2 | −0.57 |
| 32 | OMD | Osteomodulin | −0.57 |
| 41 | CHL1 | Neural cell adhesion molecule L1- | −0.56 |
| | | like protein | |
| 35 | EMR2 | EGF-like module-containing mucin- | −0.53 |
| | | like hormone receptor-like 2 | |
| 33 | IBSP | Bone sialoprotein 2 | −0.52 |
| 34 | CA6 | Carbonic anhydrase 6 | −0.52 |
| 30 | CLEC11A | Stem Cell Growth Factor-alpha | −0.46 |

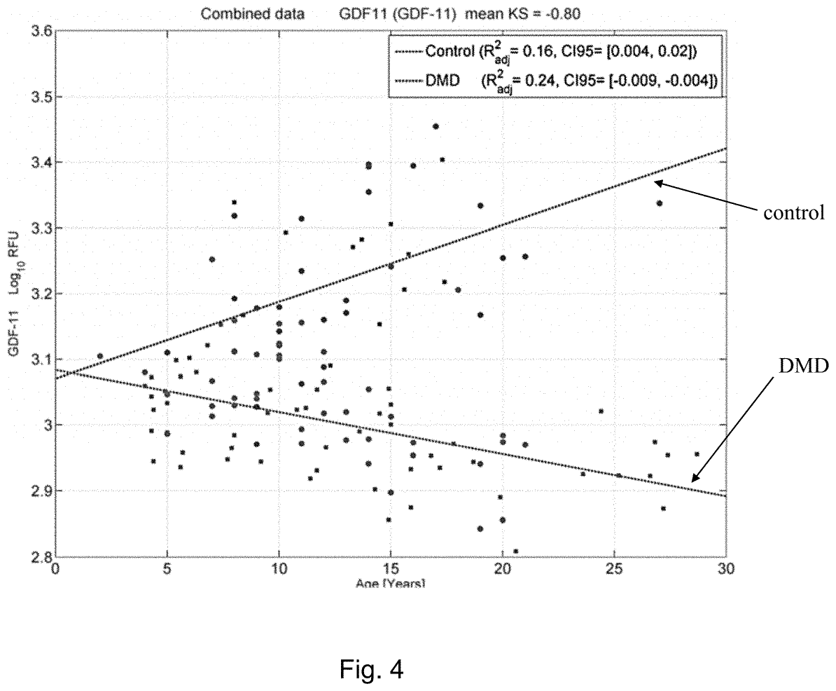

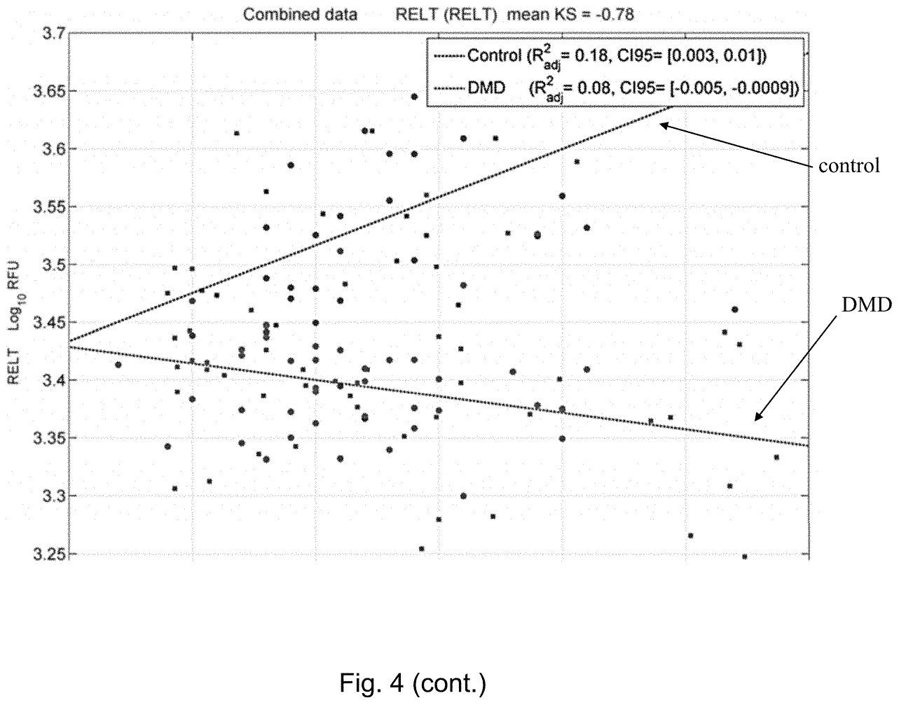

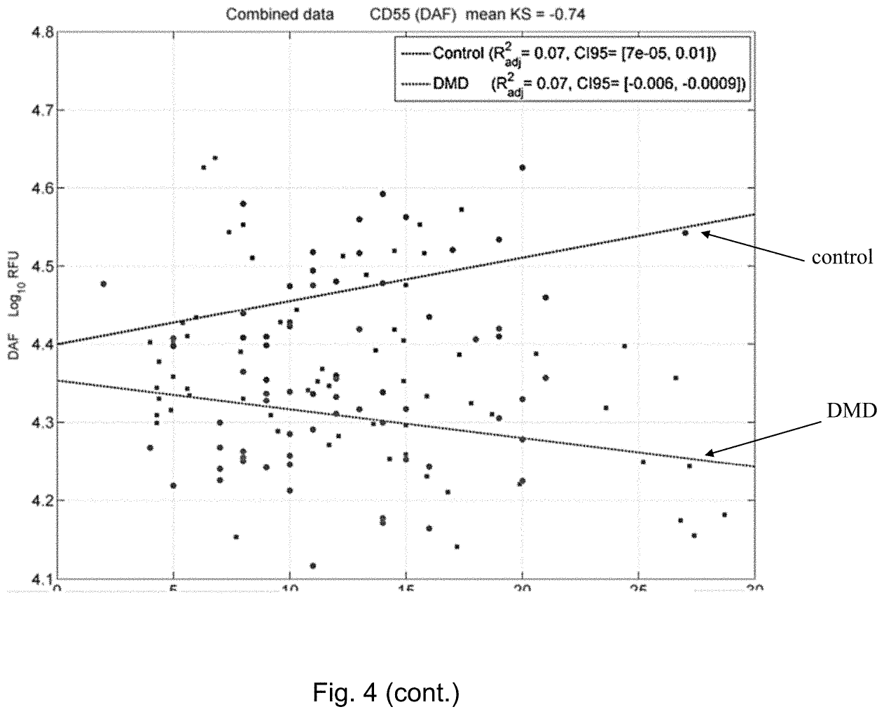

Pictographs were generated plotting the relative protein expression levels (RFU) vs. age (years) of subjects in both non-DMD and DMD boys. Proteins that are different between the control and the DMD subjects are shown in FIG. 4, where the protein decreases in the DMD subject while the same protein increases in the control.

Several animal models find use with the methods and compositions of the invention for identifying, modulating and monitoring drug targets in muscular disease. Male mice (e.g., MDx strains) have been maintained without a functional dystrophin. While these mice are not normal, the phenotype is not as severe as the phenotypes of DMD patients. The MDx mouse model becomes more severe and more like the human disease when a second knock-out is added to the dystrophin mutation (a common second mutation is in the utrophin gene). Thus, in one embodiment, GDF-11 can be administered to subject (e.g. mouse model of DMD) in order to ameliorate the symptoms of the subject (e.g., DMD symptoms of the MDx mouse and MDx-utrophin-less mouse. One of ordinary skill in the art knows well method for identifying a therapeutically effective dose. For example, it is possible to first analyze the required GDF-11 injection doses and injection schedule to maintain the circulating GDF-11 concentration at or near a wild-type level, and the determined dose could be used in the dystrophin and dystrophin-utrophin models. In addition, dog and pig dystrophin knock-outs can also be treated with injected GDF-11.

For humans, dosing pharmacokinetics and safety can be to be established. After preclinical safety/toxicity experiments have been completed to regulatory standards, a drug concentration is identified at which toxicity starts, and the target organs for toxicity identified. In one non-limiting example, human experiments are performed in single escalating dose experiments followed by multiple dose escalation experiments, usually in healthy volunteers although in this case it might be better done in DMD subjects depending on discussion with an IRB and with parent organizations because the pharmacokinetics (PK) in 18-45 year old healthy volunteers might be different. If required by such discussions, the PK experiments might need to be performed in healthy adults first and then confirmed in smaller groups of DMD children. For single dose, groups of 8 subjects (randomized to 8 active and 2 placebo per group) receive a subcutaneous and/or intramuscular injection. Blood samples are taken in a time series, typically at 0, 0.5, 1, 2, 4, 8, 24, 48 and a few days after the injection. Doses would be calculated using the mouse pharmacology and toxicity data to start at a level below any active level, and the PK and safety checked in each group before the next escalation. Subsequent groups often go up in half log dose steps until adverse effects are experienced or until a predefined stopping rule for a concentration. Typically 6 or more dose escalations are performed before a limiting adverse effect but this can be dependent upon the pharmacology.

Multiple dose studies are similar in group size and usually last 2 weeks to establish safety and steady-stake PK. These studies may use the single dose experiments' information as a starting point so the initial dose is likely to be higher. Using the PK results from single dose, a dosing regimen can be defined which is likely to achieve a target concentration or which ensures that it does not fall below a defined trough. This may be once, twice or three times a day. If there is uncertainty, the multiple dose experiment might use more than one dosing regimen. Initially if the PK is short, dosing regimens can be used which would not be practical on a large scale but which will test the hypothesis; if efficacy is achieved PK can be improved and regimens made more practical through slow release formulations.

Efficacy experiments can be performed in subjects with DMD using the regimens identified in the multiple dose PK study which achieved the target concentration (e.g. matching the normal concentration or higher). Typically a phase IIa efficacy experiment would test placebo plus 2-3 doses and dosing regimens. Groups may be of the order of 20 subjects each, selected to be early enough in the disease such that improvement is possible, and the study duration would be estimated to be long enough to see trends efficacy differences, not necessarily with each group reaching statistically significant—this may be 3-6 months or an adaptive design could be used where a data safety monitoring board lets the study continue until either futility or a difference is apparent. Metrics for efficacy may include 6 minute walk, muscle MRI, muscle biopsy and blood based biomarkers using SOMAscan and/or immunoassays. Trends in the right direction would lead to a phase IIb program which would use the phase IIa metrics to define a statistically powered size and duration. If the dosing regimen required is impractical, slow release formulations would be developed, go through the single and multiple dose PK and then into phase IIb.

### Example 3

Table 13 shows a summary of the fold expression difference in protein levels of the metalloproteinase (MMP) family members from tumor tissue versus healthy adjacent tissue for about 258 subjects with lung cancer (categorized as adenocarcinoma, squamous cell, carcinosarcoma, large cell, mucoepidermoid, spindle cell, benign, pleomorphic carcinoma, pleomorphic-adenocarcinoma, and benign with history of cancer). Individual subjects, irrespective of the specific lung cancer diagnosis, show differential MMP expression levels (overexpressed or underexpressed in tumors). The drug marimastat antagonizes MMP family members, and therefore is useful in treating cancer having one or more overexpressed MMPs. Preclinical studies showed that antagonizing MMP function or expression inhibits tumor growth (e.g., in breast cancer models).

TABLE 13
Summary of the different MMP family members and the number
of subjects having an expression level difference of four
fold or greater based on tumor tissue proteins levels
versus healthy adjacent tissue protein levels.

| | Total Subjects | Malignant Tumor | Benign Tumor |
|---|---|---|---|
| MMP | Havingat Least | Expression | Expression |
| Family | a 4-fold Expression | Levels at | Levels of at |
| Member | Difference | Least 4-fold | Least 4-fold |
| MMP-12 | 164 | 152 | 12 |
| MMP-1 | 123 | 116 | 7 |
| MMP-7 | 82 | 82 | 0 |
| MMP-9 | 38 | 34 | 4 |
| MMP-13 | 33 | 33 | 0 |
| MMP-8 | 32 | 31 | 1 |
| MMP-10 | 16 | 15 | 1 |
| MMP-2 | 9 | 7 | 2 |

In this study, no correlation was found with the specific lung cancer diagnosis, the staging of the cancer, the sex of the patient or the genetic information (e.g., gene mutation; several subjects had the BRAF, EGFR or KRAS mutation). The independence of the proteomic information, specifically for the MMP family members, may be informative as to the treatment regime that should be used for each individual.

A recent phase III clinical trial testing the efficacy of marimastat (MMP antagonist) in subjects having metastatic breast cancer showed that there was no significant difference between the marimastat treated subjects and those that received the placebo. In general, the conclusion from the trial was that marimastat was not effective in stopping and/or slowing breast cancer disease progression.

While the proteomic data summarized in Table 13 was derived from lung cancer patients, the observed heterogeneity of the MMP family members in these lung cancer subjects may be indicative of what may be observed in other cancer types (e.g., breast cancer). Accordingly, this heterogeneity may be, in part, the reason why certain anti-cancer drugs and/or treatments result in heterogeneous outcomes and/or insignificant efficacy. In this context, one may propose that treatment regimens for cancer patients and/or patients in clinical trials may be stratified based on individualized proteomic profiles, in place of, or in addition to, standard pathology and/or genetic testing. Thus, applying this reasoning to the phase III clinical trial for marimastat with breast cancer patients discussed previously, these patients could have been selected for treatment with marimastat based on the overexpression levels of MMP family members, rather than standard diagnostic methods. For lung cancer patients, the same treatment selection and/or clinical trial stratification could be applied. In effect, treatment regimens and/or clinical trial stratifications could be selected based on the expression levels of a particular protein or set of proteins whereby a 4, 10, 20 or 50-fold difference between tumor protein levels and healthy tissue levels would indicate whether an individual is likely to respond to treatment with a particular drug, such as a drug that targets (e.g., antagonizes) the protein with the elevated expression levels.

### Example 4

Table 14 provides a list of drug names that target specific proteins. Each row provides the drug-protein association or where the protein target for the drug corresponds (corresponds in the context of table 14 indicates that the protein shares the same row with the drug name of the table. This table may be used as a reference for developing a personalized treatment plan based on aberrant protein expression in an individual. For example, the reference table may be used where an individual may suffer from specific condition or disease and have up-regulated levels of Serine/threonine-protein kinase Chk1 by about 4, 10, 20 or 50-fold relative to a reference control protein level.

Thus, in one embodiment a method for selecting a subject for treatment with a drug the method comprising, detecting the level of at least one protein from Table 14 from a biological sample from the subject, determining the fold difference of the level of the at least one protein from table 14 form the biological sample compared to a reference control sample, selecting the subject for treatment with a drug from table 14 that corresponds to the at least on protein from table 14, wherein the subject is treated with the drug selected from table 14 when the fold difference of the level of the at least one protein from table 14 is at least 4-fold, 10-fold, 15-fold, 20-fold, 25-fold, 30-fold, 35-fold, 40-fold, 45-fold or 50-fold from the biological sample compared to the reference control, and wherein the subject is in need of treatment and is administered the drug for treatment based on the fold difference of the level of the at least one protein from Table 14.

TABLE 14
List of drugs that target proteins

| Protein Name | Drug Name | TTDC ID | TYPE | UniProt |
|---|---|---|---|---|
| Serine/threonine-protein | 2- | TTDC00005 | Clinical trial | O14757 |
| kinase Chk1 | (cyclohexylamino)benzoic, | | target | |
| Serine/threonine-protein | 2-(1H-pyrazol-3-yl)-1H- | TTDC00006 | Clinical trial | O14965 |
| kinase 6 | benzimidazole, | | target | |
| Serine/threonine-protein | 2-(4-Phenoxy-phenyl)-1H- | TTDC00010 | Discontinued | O96017 |
| kinase Chk2 | benzoimidazol-5-ylamine, | | target | |
| Plasma kallikrein | (3,4-dichlorophenyl)(1H- | TTDC00013 | Successful | P03952 |
| | pyrazol-1-yl)methanone, | | target | |
| Protein kinase C gamma | (−)-Cercosporamide, | TTDC00015 | Clinical trial | P05129 |
| type | | | target | |
| Heat shock protein HSP | 17-Dmag, | TTDC00018 | Clinical trial | P07900 |
| 90 | | | target | |
| Cathepsin G | Aloxistatin, | TTDC00019 | Clinical trial | P08311 |
| | | | target | |
| Integrin alpha-5 | c(-GRGDfL-), | TTDC00020 | Clinical trial | P08648 |
| | | | target | |
| Lysosomal alpha- | (−)-uniflorine, | TTDC00023 | Clinical trial | P10253 |
| glucosidase | | | target | |
| Basic fibroblast growth | 2-(1H-indazol-3-yl)-1H- | TTDC00024 | Clinical trial | P11362 |
| factor receptor 1 | benzo[d]imidazole, | | target | |
| Interleukin-2 receptor | | TTDC00026 | Clinical trial | P14784 |
| subunit beta | | | target | |
| Integrin beta-7 | TR-14035, | TTDC00031 | Clinical trial | P26010 |
| | | | target | |
| Tyrosine-protein | Sodium, | TTDC00032 | Clinical trial | P29350 |
| phosphatase non-receptor | | | target | |
| type 6 | | | | |
| Interleukin-12 | STA-5326, | TTDC00033 | Successful | P29459 |
| | | | target | |
| MAP kinase p38 | 4,5,6,7- | TTDC00044 | Clinical trial | P53778 |
| | tetrabromobenzotriazole, | | target | |
| Ephrin type-B receptor 4 | TG-100435, | TTDC00045 | Clinical trial | P54760 |
| | | | target | |
| Induced myeloid leukemia | ALTENUSIN, | TTDC00048 | Clinical trial | Q07820 |
| cell differentiation protein | | | target | |
| Mcl-1 | | | | |
| Carboxypeptidase B2 | (+/−)-5-amino-2- | TTDC00053 | Discontinued | Q96IY4 |
| | (mercaptomethyl)pentanoic, | | target | |
| Cathepsin S | 2-[(2′,3′,4′- | TTDC00067 | Discontinued | P25774 |
| | TRIFLUOROBIPHENYL- | | target | |
| | 2-YL)OXY]ETHANOL, | | | |
| 92 kDa type IV | (+/−)5-(biphenyl-4-yl)-3- | TTDC00076 | Discontinued | P14780 |
| collagenase | hydroxypentanoic, | | target | |
| Protein kinase C, delta | 13-Acetylphorbol, | TTDC00077 | Clinical trial | Q05655 |
| type | | | target | |
| Interleukin-4 receptor | | TTDC00081 | Clinical trial | P24394 |
| alpha chain | | | target | |
| C1 esterase | C1-INH, | TTDC00085 | Successful | P09871 |
| | | | target | |
| Thromboxane-A synthase | 2-(10-Imidazol-1-yl-decyl)- | TTDC00086 | Clinical trial | P24557 |
| | isoindole-1,3-dione, | | target | |
| Cell division protein | (2′Z,3′E)-5-Chloro-5′- | TTDC00088 | Clinical trial | P24941 |
| kinase 2 | chloro-indirubin-3′-oxime, | | target | |
| Purine nucleoside | (+/−)-5′-deoxy-4′-fluoro-5′- | TTDC00091 | Clinical trial | P00491 |
| phosphorylase | methylthio-DADMe-ImmH, | | target | |
| E-selectin | 1na, | TTDC00098 | Clinical trial | P16581 |
| | | | target | |
| Hypoxia-inducible factor 1 | HIF-1alpha, | TTDC00101 | Clinical trial | Q16665 |
| alpha | | | target | |
| Mitogen-activated protein | 2,6-Dihydroanthra/1,9- | TTDC00102 | Clinical trial | P45983 |
| kinase 8 | Cd/Pyrazol-6-One, | | target | |
| Macrophage migration | 3,4-Dihydroxycinnamic, | TTDC00103 | Clinical trial | P14174 |
| inhibitory factor | | | target | |
| Von Willebrand factor | Auryntricarboxylic, | TTDC00108 | Clinical trial | P04275 |
| | | | target | |
| STAT-1 transcription | AVT-02, | TTDC00113 | Clinical trial | P42224 |
| factor | | | target | |
| Receptor protein-tyrosine | CI-1033, | TTDC00114 | Discontinued | Q15303 |
| kinase erbB-4 | | | target | |
| Platelet-activating factor | (1R)-1,2,2- | TTDC00116 | Clinical trial | Q13093 |
| acetylhydrolase | TRIMETHYLPROPYL, | | target | |
| Cellular tumor antigen p53 | 1-(9-ethyl-9H-carbazol-3- | TTDC00118 | Clinical trial | P04637 |
| | yl)-N-methylmethanamine, | | target | |
| Transcription factor AP-1 | PNRI-299, | TTDC00119 | Clinical trial | P05412 |
| | | | target | |
| Leukotriene B4 receptor 1 | (3S,4R)-3-Benzyl-7- | TTDC00129 | Clinical trial | Q15722 |
| | isopropyl-chroman-4-ol, | | target | |
| Leukotriene A-4 hydrolase | (4-(thiophen-2- | TTDC00130 | Clinical trial | P09960 |
| | yl)phenyl)methanamine, | | target | |
| Interleukin-7 receptor | | TTDC00136 | Clinical trial | P16871 |
| alpha chain | | | target | |
| Neural-cadherin | | TTDC00137 | Clinical trial | P19022 |
| | | | target | |
| Serine/threonine protein | AT-9283, | TTDC00139 | Clinical trial | Q96GD4 |
| kinase 12 | | | target | |
| Phosphatidylinositol-4,5- | 2-(4-Morpholinyl)-8- | TTDC00140 | Clinical trial | P48736 |
| bisphosphate 3-kinase | Phenyl-4h-1-Benzopyran-4- | | target | |
| catalytic subunit, gamma | One, | | | |
| isoform | | | | |
| Hexokinase D | Beta-D-Glucose, | TTDC00141 | Clinical trial | P35557 |
| | | | target | |
| mRNA of Clusterin | | TTDC00142 | Clinical trial | P10909 |
| | | | target | |
| Fructose-1,6- | 1-(2-mercaptoethyl)-3-(m- | TTDC00152 | Clinical trial | P09467 |
| bisphosphatase | tolylsulfonyl)urea, | | target | |
| Tyrosine-protein kinase | ELLAGIC, | TTDC00156 | Clinical trial | P43405 |
| SYK | | | target | |
| Serine/threonine-protein | BI, | TTDC00160 | Clinical trial | P53350 |
| kinase PLK1 | | | target | |
| Angiopoietin 1 receptor | (4-Phenoxy-phenyl) - | TTDC00161 | Discontinued | Q02763 |
| | quinazolin-4-yl-amine, | | target | |
| Protein kinase C, beta type | (−)-Cercosporamide, | TTDC00163 | Clinical trial | P05771 |
| | | | target | |
| Cell division control | (2,6-Diamino-pyridin-3-yl)- | TTDC00166 | Clinical trial | P06493 |
| protein 2 homolog | phenyl-methanone, | | target | |
| Antiapoptotic protein | 4′-FLUORO-1,1′- | TTDC00168 | Clinical trial | Q07817 |
| BCL-XL | BIPHENYL-4- | | target | |
| | CARBOXYLIC, | | | |
| PDE4 | (R)-Rolipram, | TTDC00170 | Clinical trial | Q08499 |
| | | | target | |
| Interleukin-13 | Anti-IL13, | TTDC00177 | Clinical trial | P35225 |
| | | | target | |
| Protein kinase C, theta | 2,3,3-Triphenyl- | TTDC00178 | Clinical trial | Q04759 |
| type | acrylonitrile, | | target | |
| Amyloid beta A4 protein | 1,6-Bis(4′-hydroxyphenyl)- | TTDC00180 | Successful | P05067 |
| | hexa-1,3,5-triene, | | target | |
| Protein kinase C, alpha | (−)-Cercosporamide, | TTDC00182 | Clinical trial | P17252 |
| type | | | target | |
| Interleukin-9 | MEDI-528, | TTDC00186 | Clinical trial | P15248 |
| | | | target | |
| Tumor necrosis factor receptor superfamily member 16 | | TTDC00189 | Clinical trial | P08138 |
| | | | target | |
| Protein-tyrosine | 1,2,5-THIADIAZOLIDIN- | TTDC00191 | Clinical trial | P18031 |
| phosphatase, non-receptor | 3-ONE-1,1-DIOXIDE, | | target | |
| type 1 | | | | |
| mRNA of Intercellular | A-286982, | TTDC00192 | Clinical trial | P05362 |
| adhesion molecule-1 | | | target | |
| Mitogen-activated protein | (5-amino-1-phenyl-1H- | TTDC00201 | Clinical trial | Q16539 |
| kinase 14 | pyrazol-4- | | target | |
| | yl)phenylmethanone, | | | |
| Ubiquitin-protein ligase | R7112, | TTDC00206 | Successful | Q00987 |
| E3 Mdm2 | | | target | |
| Angiopoietin-2 | AMG, | TTDC00210 | Clinical trial | O15123 |
| | | | target | |
| Connective tissue growth | FG-3019, | TTDC00213 | Clinical trial | P29279 |
| factor | | | target | |
| Interleukin-17 | AIN457, | TTDC00214 | Clinical trial | Q16552 |
| | | | target | |
| Tumor necrosis factor receptor superfamily member 4 | | TTDC00219 | Clinical trial | P43489 |
| | | | target | |
| Sodium-and chloride-dependent glycine transporter 1 | | TTDC00227 | Clinical trial | P48067 |
| | | | target | |
| Interleukin-1 receptor, | | TTDC00234 | Clinical trial | P27930 |
| type II | | | target | |
| Bcl-2-like protein 2 | ABT-263, | TTDC00244 | Clinical trial | Q92843 |
| | | | target | |
| Synaptic vesicle | Brivaracetam, | TTDC00246 | Clinical trial | Q7L0J3 |
| glycoprotein 2A | | | target | |
| Mucosal addressin cell | | TTDC00248 | Clinical trial | Q13477 |
| adhesion molecule 1 | | | target | |
| Pigment epithelium- | AdPEDR, | TTDC00252 | Clinical trial | P36955 |
| derived factor | | | target | |
| Ciliary neurotrophic factor | | TTDC00257 | Clinical trial | P26992 |
| receptor alpha | | | target | |
| Beta-2-glycoprotein 1 | Alpha-D-Mannose, | TTDC00264 | Clinical trial | P02749 |
| | | | target | |
| Tumor necrosis factor receptor superfamily member 10B | | TTDC00266 | Clinical trial | O14763 |
| | | | target | |
| mRNA of Heat shock 27 | BIRB796, | TTDC00269 | Clinical trial | P04792 |
| kDa protein | | | target | |
| Myc proto-oncogene | TWS-119, | TTDC00271 | Clinical trial | P01106 |
| protein | | | target | |
| Transforming growth | | TTDC00272 | Clinical trial | P61812 |
| factor beta 2 | | | target | |
| Baculoviral IAP repeat- | Terameprocol, | TTDC00273 | Clinical trial | O15392 |
| containing protein 5 | | | target | |
| Alpha platelet-derived | (1H-indol-2-yl)(5-methoxy- | TTDC00311 | Discontinued | P16234 |
| growth factor receptor | 1H-indol-2-yl)methanone, | | target | |
| Nicotinic acid receptor | 1H-Pyrazole-3-carboxylic, | TTDC00317 | Successful | Q8TDS4 |
| | | | target | |
| mRNA of copper zinc superoxide dismutase 1 | | TTDC00325 | Clinical trial | P00441 |
| | | | target | |
| Complement factor D | | TTDC00326 | Clinical trial | P00746 |
| | | | target | |
| mRNA of Factor XI | | TTDC00330 | Clinical trial | P03951 |
| | | | target | |
| Apolipoprotein B-100 | SPC4955, | TTDC00331 | Successful | P04114 |
| | | | target | |
| mRNA of VEGFR1 | (2-Methoxy-phenyl)-(5- | TTDC00334 | Clinical trial | P17948 |
| | phenyl-oxazol-2-yl)-amine, | | target | |
| mRNA of connective | | TTDC00335 | Clinical trial | P29279 |
| tissue growth factor | | | target | |
| CD70 | | TTDC00337 | Clinical trial | P32970 |
| | | | target | |
| Activin receptor-like | ACE-041, | TTDC00338 | Clinical trial | P37023 |
| kinase-1 | | | target | |
| Nectin-4 | | TTDC00343 | Clinical trial | Q96NY8 |
| | | | target | |
| mRNA of growth | | TTDC00345 | Clinical trial | P10912 |
| hormone receptor | | | target | |

### REFERENCES

Kris M G, Johnson B E, Berry L D, Kwiatkowski D J, Iafrate A J, Wistuba, I I, Varella-Garcia M, Franklin W A, Aronson S L, Su P F, Shyr Y, Camidge D R, Sequist L V, Glisson B S, Khuri F R, Garon E B, Pao W, Rudin C, Schiller J, Haura E B, Socinski M, Shirai K, Chen H, Giaccone G, Ladanyi M, Kugler K, Minna J D, Bunn P A: Using multiplexed assays of oncogenic drivers in lung cancers to select targeted drugs. JAMA 2014, 311:1998-2006.
Gray S W, Hicks-Courant K, Cronin A, Rollins B J, Weeks J C: Physicians' attitudes about multiplex tumor genomic testing. Journal of Clinical Oncology 2014, 32:1317-1323.
Lex, Geoff Baird, Dan Theodorescu, and David Cooper have to help with the last section, along with Steve Williams and Fintan Steele
Mehan M R, Ayers D, Thirstrup D, Xiong W, Ostroff R M, Brody E N, Walker J J, Gold L, Jarvis T C, Janjic N, Baird G S, Wilcox S K: Protein signature of lung cancer tissues. PLoSOne 2012, 7:e35157.
Doebele R C, Lu X, Sumey C, Maxson D A, Weickhardt A J, Oton A B, Bunn P A, Jr., Baron A E, Franklin W A, Aisner D L, Varella-Garcia M, Camidge D R: Oncogene status predicts patterns of metastatic spread in treatment-naive nonsmall cell lung cancer. Cancer 2012, 118:4502-4511.
Su Z, Dias-Santagata D, Duke M, Hutchinson K, Lin Y L, Borger D R, Chung C H, Massion P P, Vnencak-Jones C L, Iafrate A J, Pao W: A platform for rapid detection of multiple oncogenic mutations with relevance to targeted therapy in non-small-cell lung cancer. The Journal of molecular diagnostics: JMD 2011, 13:74-84.
Gold L, Ayers D, Bertino J, Bock C, Bock A, Brody E N, Carter J, Dalby A B, Eaton B E, Fitzwater T, Flather D, Forbes A, Foreman T, Fowler C, Gawande B, Goss M, Gunn M, Gupta S, Halladay D, Heil J, Heilig J, Hicke B, Husar G, Janjic N, Jarvis T, Jennings S, Katilius E, Keeney T R, Kim N, Koch T H, et al: Aptamer-based multiplexed proteomic technology for biomarker discovery. PLoSOne 2010, 5:e15004.
Mehan M R, Ostroff R, Wilcox S K, Steele F, Schneider D, Jarvis T C, Baird G S, Gold L, Janjic N: Highly multiplexed proteomic platform for biomarker discovery, diagnostics, and therapeutics. AdvExpMedBiol 2013, 735:283-300.
Vaught J D, Bock C, Carter J, Fitzwater T, Otis M, Schneider D, Rolando J, Waugh S, Wilcox S K, Eaton B E: Expanding the chemistry of DNA for in vitro selection. JAmChemSoc 2010, 132:4141-4151.
Kraemer S, Vaught J D, Bock C, Gold L, Katilius E, Keeney T R, Kim N, Saccomano N A, Wilcox S K, Zichi D, Sanders G M: From SOMAmer-based biomarker discovery to diagnostic and clinical applications: a SOMAmer-based, streamlined multiplex proteomic assay. PLoSOne 2011, 6:e26332.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, in vitro fertilization, development, or related fields are intended to be within the scope of the following claims.

1. A method for identifying protein targets, comprising:
a) assaying a biological sample from a subject diagnosed with a disease to identify altered levels of one or more proteins relative to the level of said protein in a reference sample; and
b) identifying one or more treatments that targets one or more of said proteins with altered expression.

2. The method of claim 1, wherein said proteins are selected from AGER, THBS2, CA3, MMP12, MMP-1, MMP-7, MMP-9, MMP-13, MMP-8, MMP-10, MMP-2, PIGR, DCN, PGAM1, CD36, FABP, ACP5, CCDC80, PPBP, LYVE1, STC1, SPON1, IL17RC, MMP1, CA1, SERPINC1, TPSB2, CKB/CKBM, NAMPT/PBEF, PPBP/CTAPIII, F9, DCTPP1, F5, SPOCK2, CAT, PF4, MDK, BGN, CKM, POSTN, PGLYRP1, and CXCL12.

3. The method of claim 1, wherein said reference sample is sample of normal tissue from said subject, or a population average of normal tissue.

4. (canceled)

5. (canceled)

6. The method of claim 1 further comprising administering said one or more treatments to said subject.

7. (canceled)

8. The method of claim 1, wherein said disease is selected from the group consisting of a cancer, a metabolic disorder, an inflammatory disease and an infectious disease.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A method for selecting a subject for treatment with a drug, the method comprising:
a) detecting the level of a matrix metalloproteinase (MMP) protein from a biological sample from a subject, wherein the biological sample is a sample from diseased tissue or diseased cells from the subject;
b) determining a fold difference of the level of the MMP protein from the biological sample compared to a normal biological sample of the same tissue or cell type from the same subject;
c) selecting the subject for treatment with a drug based on the fold difference of the level of the MMP protein, wherein the subject is treated with the drug when the fold difference of the level of the MMP protein is at least 4-fold, 10-fold, 15-fold, 20-fold, 25-fold, 30-fold, 35-fold, 40-fold, 45-fold or 50-fold from the biological sample compared to the normal biological sample, and wherein the subject is in need of treatment and is administered the drug for treatment based on the fold difference of the level of the MMP protein.

29. The method of claim 28, wherein the MMP protein is MMP12, MMP-1, MMP-7, MMP-9, MMP-13, MMP-8, MMP-10, MMP-2 or a combination thereof.

30. The method of claim 28, wherein the drug is marimastat.

31. (canceled)

32. (canceled)

33. (canceled)

34. (canceled)

35. (canceled)

36. (canceled)

37. (canceled)

38. A method for selecting a subject for a clinical trial, the method comprising:
a) detecting the level of a protein from a biological sample from a subject;
b) determining a fold difference of the level of the protein from the biological sample compared to a normal biological sample from the same subject; and
c) selecting the subject for the clinical trial or excluding the subject from the clinical trial based on the fold difference of the level of the protein, wherein the subject is included in the clinical trial when the fold difference of the level of the protein is at least 4-fold, 10-fold, 15-fold, 20-fold, 25-fold, 30-fold, 35-fold, 40-fold, 45-fold or 50-fold from the biological sample compared to the normal biological sample.

39. The method of claim 38, wherein the protein is MMP12, MMP-1, MMP-7, MMP-9, MMP-13, MMP-8, MMP-10, MMP-2 or a combination thereof.

40. The method of claim 38, wherein the drug is marimastat.

41. The method of claim 38, wherein the biological sample is a tumor sample, serum sample, plasma sample, urine sample, blood sample, saliva sample, tissue sample, cell sample or a combination thereof.

42. The method of claim 38, wherein the detecting is performed with an aptamer, antibody and/or mass spectrometry.

43. The method of claim 38, wherein the normal biological sample is the same sample type as the biological sample.

44. The method of claim 38, wherein the normal biological sample is a sample taken from the same subject at a time when the subject was not diagnosed with a disease or condition, or is a sample taken from the subject where the sample does not have the genotype and/or the phenotype of the biological sample.

45. The method of claim 38, wherein the subject has cancer.

46. The method of claim 45, wherein the cancer is leukemia, lymphoma, prostate cancer, lung cancer, breast cancer, liver cancer, colorectal cancer, kidney cancer.

47. The method of claim 46, wherein the cancer is lung cancer.

48. The method of claim 47, wherein the lung cancer is selected from non-small cell lung cancer (NSCLC), small cell lung cancer, large cell lung cancer, adenocarcinoma, squamous carcinoma, carcinosarcoma, mucoepidermoid carcinoma, spindle cell carcinoma, pleomorphic carcinoma, and pleomorphic adenomacarcinoma.
